(19) United States
(12) Patent Application Publication
(10) Pub. No.: US 2024/0287072 A1
(43) Pub. Date: Aug. 29, 2024

(54) SOLID STATE FORMS OF LUMATEPERONE SALTS AND PROCESSES FOR PREPARATION OF LUMATEPERONE AND SALTS THEREOF

(71) Applicant: TEVA PHARMACEUTICALS INTERNATIONAL GMBH, Jona (CH)

(72) Inventors: Nikolina Janton, Jakovlje (HR); Helena Ceric, Zagreb (HR); Sanja Matecic Musanic, Zagreb (HR); Natasa Mrsic, Sesvetski Kraljevec (HR); Lidija Lerman, Zagreb (HR); Tina D. Momcilovic, Sveti Ivan Zelina (HR); Ivana Sagud, Gornja Stubica (HR); Alexandr Jegorov, Dobra Voda (CZ)

(21) Appl. No.: 18/632,525

(22) Filed: Apr. 11, 2024

Related U.S. Application Data
(63) Continuation of application No. 17/297,035, filed on May 26, 2021.
(60) Provisional application No. 62/905,565, filed on Sep. 25, 2019.
(60) Provisional application No. 62/893,348, filed on Aug. 29, 2019.
(60) Provisional application No. 62/811,761, filed on Feb. 28, 2019.
(60) Provisional application No. 62/771,863, filed on Nov. 27, 2018.

(51) Int. Cl. C07D 471/16; C07C 309/29

(52) U.S. Cl. CPC C07D 471/16; C07C 309/29; C07B 2200/13

(57) ABSTRACT

The present disclosure relates to solid state forms of Lumateperone besylate. processes for preparation thereof and pharmaceutical compositions thereof.

### CROSS REFERENCE TO RELATED APPLICATIONS

This application is a continuation of U.S. application Ser. No. 17/297,035, filed on May 26, 2021, which is a National Stage of, and claims priority to and the benefit of, International Patent Application No. PCT/US2019/063519 filed on Nov. 27, 2019, which, in turn, claims the benefit of and priority to, U.S. Provisional Patent Application No. 62/771,863, filed on Nov. 27, 2018, U.S. Provisional Patent Application No. 62/811,761, filed on Feb. 28, 2019, U.S. Provisional Patent Application No. 62/893,348, filed on Aug. 29, 2019, and U.S. Provisional Patent Application No. 62/905,565, filed on Sep. 25, 2019, the entire disclosures of each of which are incorporated by reference herein.

### FIELD OF THE DISCLOSURE

The present disclosure relates to solid state forms of Lumateperone besylate, processes for preparation thereof and pharmaceutical compositions thereof.

### BACKGROUND OF THE DISCLOSURE

Lumateperone 1-(4-fluorophenyl)-4-((6bR, 10aS)-3-methyl-2,3,6b,9,10,10a-hexahydro-1H-pyrido[3′,4′:4,5]pyrrolo[1,2,3-de]quinoxalin-8(7H)-yl)butan-1-one, referred to herein as compound 1 has the following chemical structure:

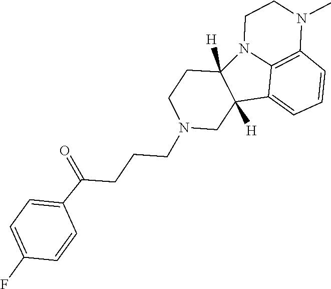 [SMILES: CN1CCN2c3c(cccc31)[C@@H]1CN(CCCC(=O)c3ccc(F)cc3)CC[C@@H]12]

Lumateperone Tosylate is under development for the treatment of central nervous system disorders including: schizophrenia, bipolar disorder, depression, sleep and behavioral disturbance in dementia, autism, and other neuropsychiatric disorders.

Lumateperone and its acceptable pharmaceutical salts are described in U.S. Pat. No. USRE39,680.

U.S. Pat. No. 8,648,077 describes polymorphs A and B of toluenesulfonic acid addition salt of lumateperone (“lumateperone tosylate”).

International Publication No. WO 2017/172784 discloses that in free base form, lumateperone is an oily, sticky solid with poor solubility, that making salts of the compound has proven to be difficult; and that the hydrochloride salt of lumateperone disclosed in U.S. Pat. No. 7,183,282 is hygroscopic and shows poor stability. WO 2017/172784 further discloses oxalate, 4-aminosalicylate and cyclamate salts of Lumateperone and crystalline forms thereof. International Publication No. WO 2017/172811 discloses cocrystals of Lumateperone with nicotinamide and isonicotinamide co-crystal forms. International Publication No. WO 2019/069591 discloses crystalline forms of Lumateperone HCl.

International Publication No. WO 2018/031535 discloses crystalline forms of Lumateperone ditosylate, International Publication No. WO 2018/071233 discloses amorphous solid dispersions of Lumateperone and International Publication No. WO 2018/189646 discloses Lumateperone tosylate in amorphous form and solid dispersions

International Publication No. WO 2000/077022 discloses the preparation of a racemate of Lumateperone and its enantiomer and isolation of Lumateperone by chiral HPLC, as depicted in scheme 1 (below).

International Publication No. WO 2008/112280 discloses a method for preparation of Lumateperone wherein intermediate resolution is performed in an earlier step and Mandelic acid is used for resolution of intermediate II, as depicted in scheme 2 (below).

Journal of Medicinal chemistry 2014, 57, 2670-2682 discloses the route depicted in scheme 3 (below).

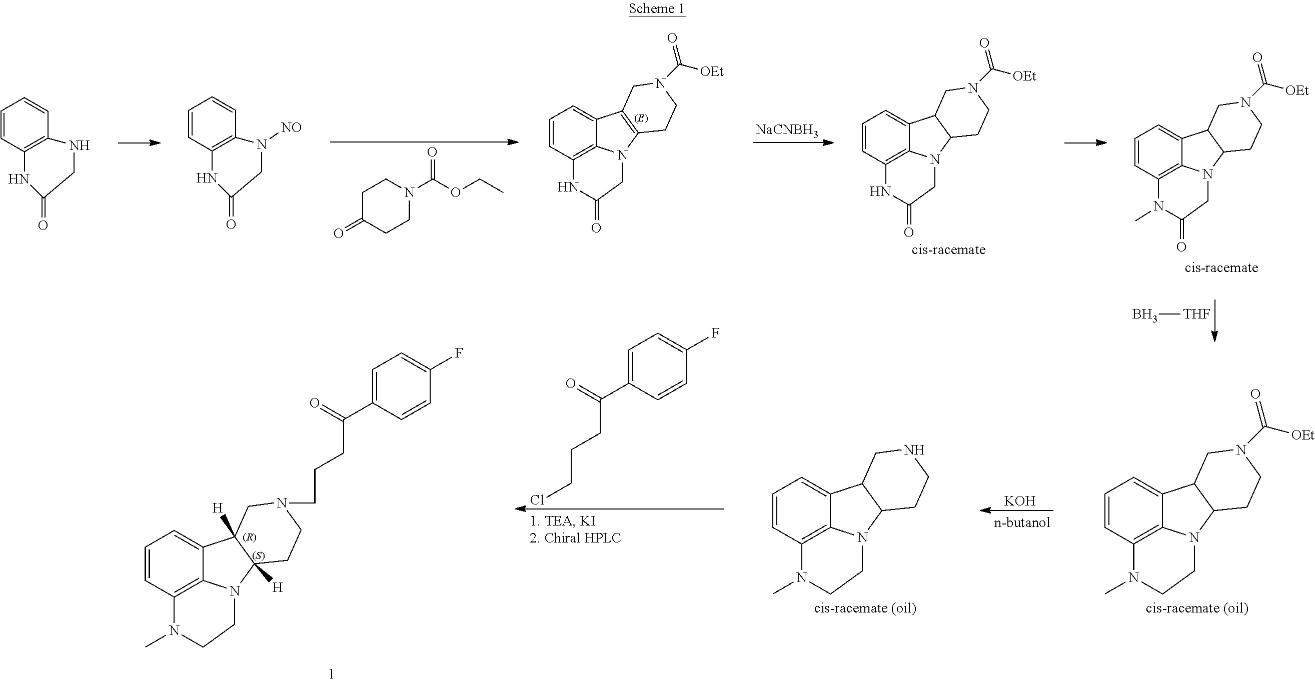

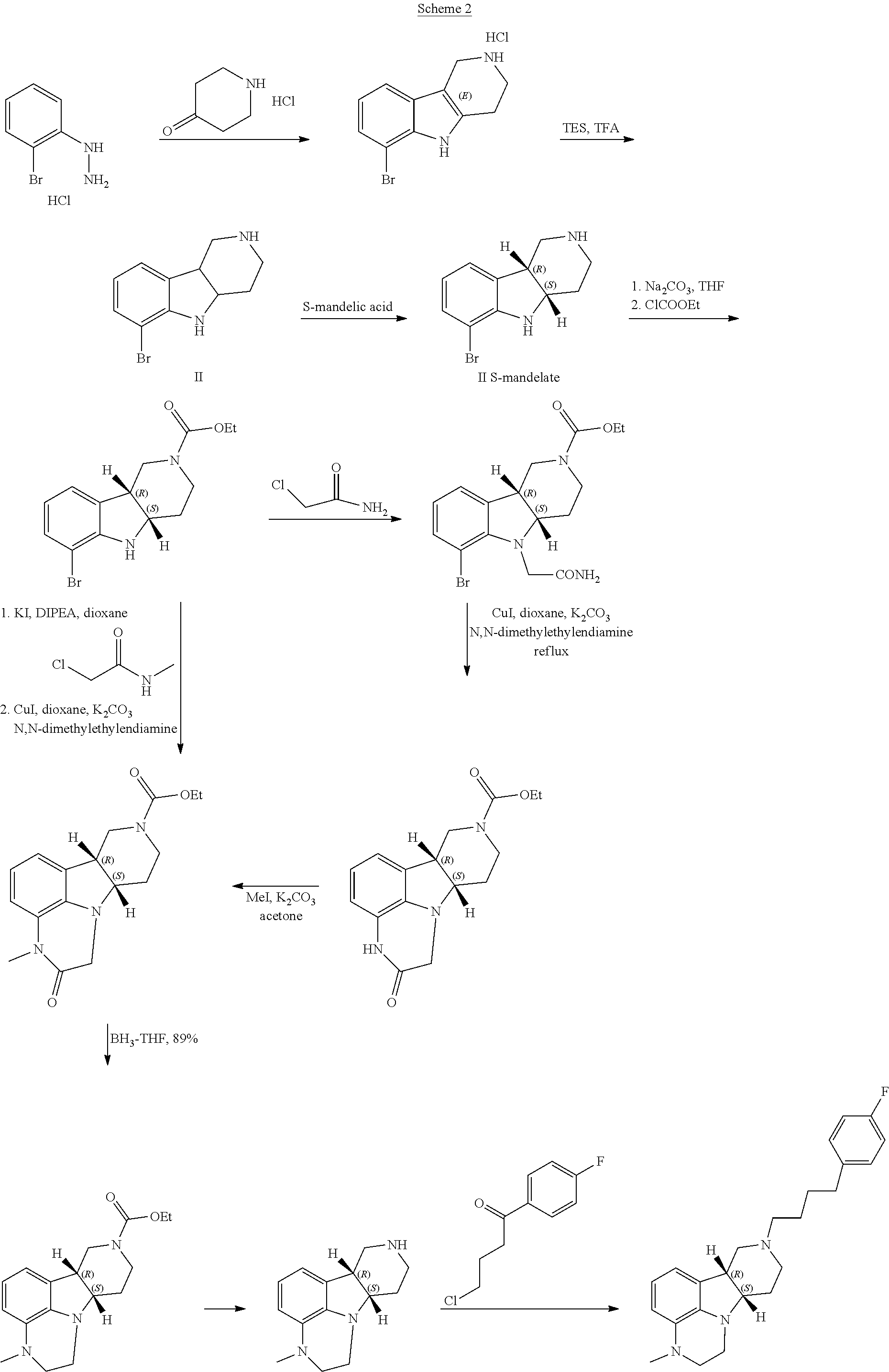

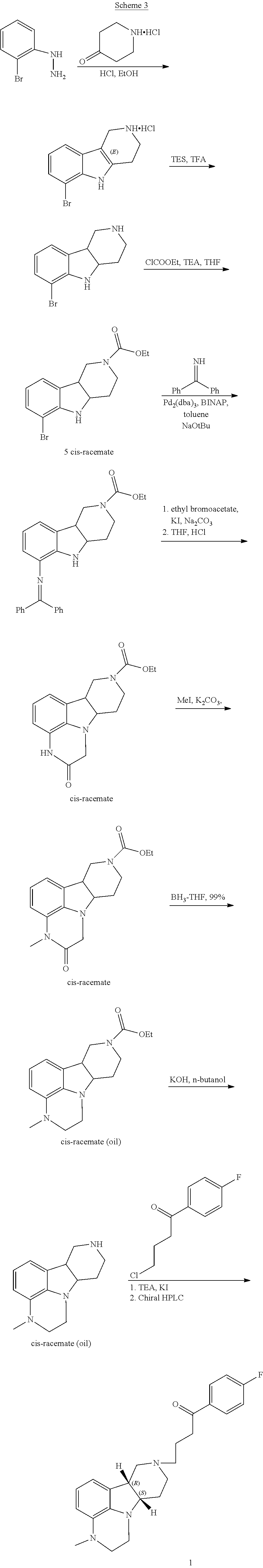

International Publication No. WO 2019/102240 discloses a process for preparation of Lumateperone wherein the cis-racemate compound of formula III

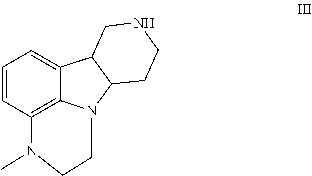 [SMILES: CN1CCN2c3c(cccc31)C1CNCCC12]

is resolved using dibenzoyl-D-tartaric acid mono(dimethylamide). The cis diastereomer (6bR, 10aS) salt is isolated and is then transformed into a base or into the dihydrochloride salt. The salt or base obtained in this way are crystallized and alkylated to afford Lumateperone and each one of these intermediate is reported to be isolated.

IPCOM000258304D and IPCOM000257454D each disclose a further route for preparation of Lumateperone.

Polymorphism, the occurrence of different crystal forms, is a property of some molecules and molecular complexes. A single compound, like Lumateperone or salt thereof, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (13C—) NMR spectrum. One or more of these techniques may be used to distinguish different polymorphic forms of a compound.

Different solid state forms (including solvated forms) of an active pharmaceutical ingredient may possess different properties. Such variations in the properties of different solid state forms and solvates may provide a basis for improving formulation, for example, by facilitating better processing or handling characteristics, improving the dissolution profile, or improving stability (polymorph as well as chemical stability) and shelf-life. These variations in the properties of different solid state forms may also provide improvements to the final dosage form, for instance, if they serve to improve bioavailability. Different solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new solid state forms and solvates of a pharmaceutical product can provide materials having desirable processing properties, such as case of handling, case of processing, storage stability, and case of purification or as desirable intermediate crystal forms that facilitate conversion to other polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional solid state forms (including co-crystals or solvated forms) of Lumateperone or salts thereof.

Efficient processes for preparation of Lumateperone in high yield and quality, and that can be utilized in industrial scale, remain desirable.

### SUMMARY OF THE INVENTION

The present disclosure relates to solid state forms of Lumateperone Besylate. The present disclosure also relates to processes for preparation thereof, and pharmaceutical compositions including the solid state forms of Lumateperone Besylate.

The present disclosure also provides uses of the said solid state forms of Lumateperone Besylate in the preparation of other solid state forms of Lumateperone Besylate, Lumateperone or other salts of Lumateperone and solid state forms thereof.

The present disclosure also provides the said solid state forms of Lumateperone Besylate for use in the preparation of other solid state forms of Lumateperone Besylate, Lumateperone or other salts of Lumateperone and solid state forms thereof.

In another embodiment, the present disclosure encompasses use of the described solid state forms of Lumateperone Besylate in the preparation of pharmaceutical compositions and/or formulations, optionally for the treatment of treatment of central nervous system disorders such as schizophrenia, bipolar disorder, depression, sleep and behavioral disturbance in dementia, autism, and other neuropsychiatric disorders.

In another embodiment, the present disclosure encompasses the described solid state forms of Lumateperone Besylate for use in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

The present disclosure further provides pharmaceutical compositions including the solid state forms of Lumateperone Besylate according to the present disclosure.

In yet another embodiment, the present disclosure encompasses pharmaceutical formulations including the described solid state forms of Lumateperone Besylate and at least one pharmaceutically acceptable excipient.

The present disclosure encompasses processes to prepare said pharmaceutical formulations of Lumateperone Besylate including combining the described solid state forms of Lumateperone Besylate and at least one pharmaceutically acceptable excipient.

The solid state forms defined herein as well as the pharmaceutical compositions or formulations of the improved solid state form of Lumateperone Besylate can be used as medicaments, particularly for the treatment of central nervous system disorders, as specified above.

The present disclosure also provides methods of treating particularly central nervous system disorders, as specified above, including administering a therapeutically effective amount of the improved solid state form of Lumateperone Besylate of the present disclosure, or at least one of the herein described pharmaceutical compositions or formulations, to a subject suffering from the above specified diseases, or otherwise in need of the treatment.

The present disclosure also provides uses of the improved solid state form of Lumateperone Besylate of the present disclosure, or at least one of the above pharmaceutical compositions or formulations for the manufacture of medicaments, particularly for medicaments, particularly for the treatment of central nervous system disorders, as specified above.

The present disclosure relates to crystalline Lumateperone tosylate:R-(-)-mandelic acid and a solid state form thereof. The present disclosure also relates to processes for preparation thereof, and pharmaceutical compositions including crystalline Lumateperone tosylate:R-(-)-mandelic acid or the described solid state form thereof.

In another embodiment, the present disclosure encompasses use of crystalline Lumateperone tosylate:R-(-)-mandelic acid or the described solid state form thereof in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

In another embodiment, the present disclosure encompasses crystalline Lumateperone tosylate:R-(-)-mandelic acid or the described solid state form thereof for use in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

The present disclosure further provides pharmaceutical compositions including crystalline Lumateperone tosylate:R-(-)-mandelic acid or the described solid state form thereof.

In yet another embodiment, the present disclosure encompasses pharmaceutical formulations including crystalline Lumateperone tosylate:R-(-)-mandelic acid or the described solid state form thereof and at least one pharmaceutically acceptable excipient.

The present disclosure encompasses processes to prepare said pharmaceutical formulations of crystalline Lumateperone tosylate:R-(-)-mandelic acid including combining crystalline Lumateperone tosylate:R-(-)-mandelic acid or the described solid state form thereof and at least one pharmaceutically acceptable excipient.

Crystalline Lumateperone tosylate:R-(-)-mandelic acid or the described solid state form thereof can be used as a medicament, particularly for the treatment of central nervous system disorders, as specified above.

The present disclosure also provides methods of treatment, particularly for the treatment of central nervous system disorders, as specified above, by administering a therapeutically effective amount of crystalline Lumateperone tosylate:R-(-)-mandelic acid or of the described solid state form thereof, or at least one of the herein described pharmaceutical compositions or formulations, to a subject suffering from a central nervous system disorder or the above specified diseases, or otherwise in need of the treatment.

The present disclosure also provides uses of crystalline Lumateperone tosylate : R-(-)-mandelic acid of the present disclosure, or of the described solid state form thereof, or at least one of the above pharmaceutical compositions or formulations for the manufacture of medicaments, in embodiments for the treatment of central nervous system disorders, as specified above.

The present disclosure provides novel processes for preparation of Lumateperone or salts thereof.

The disclosure further provides novel intermediates and crystalline forms thereof that can be advantageously used for preparation of Lumateperone or salts thereof and processes for preparation of the intermediates.

In another aspect the present disclosure provides the use of any one of the novel intermediates or the crystalline forms thereof for the preparation of Lumateperone or salts thereof, or a solid state for of Lumateperone or a salt thereof.

In another aspect the present disclosure provides Lumateperone or salts thereof produced by the processes of the present disclosure.

The present disclosure also encompasses the use of the Lumateperone or salts thereof prepared by the processes of the present disclosure for the preparation of pharmaceutical compositions of Lumateperone or salts thereof.

The present disclosure includes processes for preparing the above mentioned pharmaceutical compositions. The processes include combining the Lumateperone or salt thereof prepared by the processes of the present disclosure or salts thereof with at least one pharmaceutically acceptable excipient.

Lumateperone or salts thereof prepared by the processes of the present disclosure and the pharmaceutical compositions of Lumateperone or salts thereof prepared by the processes of the present disclosure can be used as medicaments, in embodiments for the treatment of central nervous system (CNS) disorders.

The present disclosure also provides methods for the treatment of central nervous system (CNS) disorders, by administering a therapeutically effective amount of Lumateperone or salts thereof prepared by the processes of the present disclosure, or at least one of the above pharmaceutical compositions, to a subject in need of the treatment.

### BRIEF DESCRIPTION OF THE FIGURES

FIG. 1 shows an X-ray powder diffractogram (XRPD) of crystalline form A of Lumateperone Besylate.

FIG. 2 shows DSC and TGA thermograms of crystalline form A of Lumateperone Besylate.

FIG. 3 shows an X-ray powder diffractogram (XRPD) of crystalline form 1 of Lumateperone tosylate:R-(-)-mandelic acid.

FIG. 4 shows a characteristic X-ray powder diffraction pattern (XRPD) of form 1 of Benzyl 6-bromo-3,4-dihydro-1H-pyrido[4,3-b]indole-2(5H)-carboxylate (2a).

FIG. 4a shows a characteristic X-ray powder diffraction pattern (XRPD) of form 2 of Benzyl 6-bromo-3,4-dihydro-1H-pyrido[4,3-b]indole-2(5H)-carboxylate (2a).

FIG. 5 shows a characteristic X-ray powder diffraction pattern (XRPD) of form 1 of benzyl 6-bromo-5-(2-(methylamino)-2-oxoethyl)-3,4-dihydro-1H-pyrido[4,3-b]indole-2(5H)-carboxylate (3a).

FIG. 6 shows a characteristic X-ray powder diffraction pattern (XRPD) of form 1 of Benzyl 3-methyl-2-oxo-2,3,9,10-tetrahydro-1H-pyrido[3′,4′:4,5]pyrrolo[1,2,3-de]quinoxaline-8(7H)-carboxylate (4a).

FIG. 7 shows a characteristic X-ray powder diffraction pattern (XRPD) of form 1 of benzyl 3-methyl-2,3,9,10-tetrahydro-1H-pyrido[3′,4′:4,5]pyrrolo[1,2,3-de]quinoxaline-8(7H)-carboxylate (5a).

FIG. 8 shows a characteristic X-ray powder diffraction pattern (XRPD) of form 1 of (6bR,10aS)-3-methyl-2,3,6b,7,8,9,10,10a-octahydro-1H-pyrido[3′,4′:4,5]pyrrolo[1,2,3-de]quinoxaline salt with (-)-O,O′-Di-p-toluoyl- L-tartaric acid (8).

FIG. 9 shows an X-ray powder diffractogram (XRPD) of crystalline form A of Lumateperone dibenzenesulfonate obtained according to Example 12, step (B).

FIG. 10 shows DSC thermogram of crystalline form A of Lumateperone dibenzenesulfonate obtained according to Example 12, step (B).

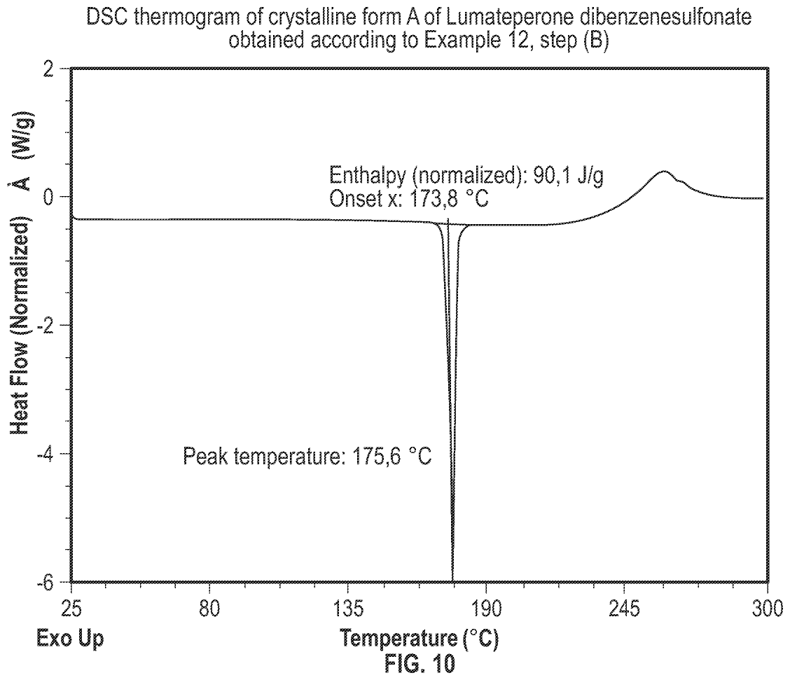

FIG. 11 shows TGA thermogram of crystalline form A of Lumateperone dibenzenesulfonate obtained according to Example 12, step (B).

FIG. 12 shows ¹³C solid state NMR spectrum of crystalline form A of Lumateperone dibenzenesulfonate , step (B).

FIG. 13 shows FTIR spectrum of crystalline form A of Lumateperone dibenzenesulfonate obtained according to Example 12, step (B).

FIG. 14 shows an X-ray powder diffractogram (XRPD) of crystalline form 1 of co-crystal of Lumateperone tosylate:R-(-)-mandelic acid obtained according to Example 12, step (D).

FIG. 15 shows DSC thermogram of crystalline form 1 of co-crystal of Lumateperone tosylate:R-(-)-mandelic acid obtained according to Example 12, step (D).

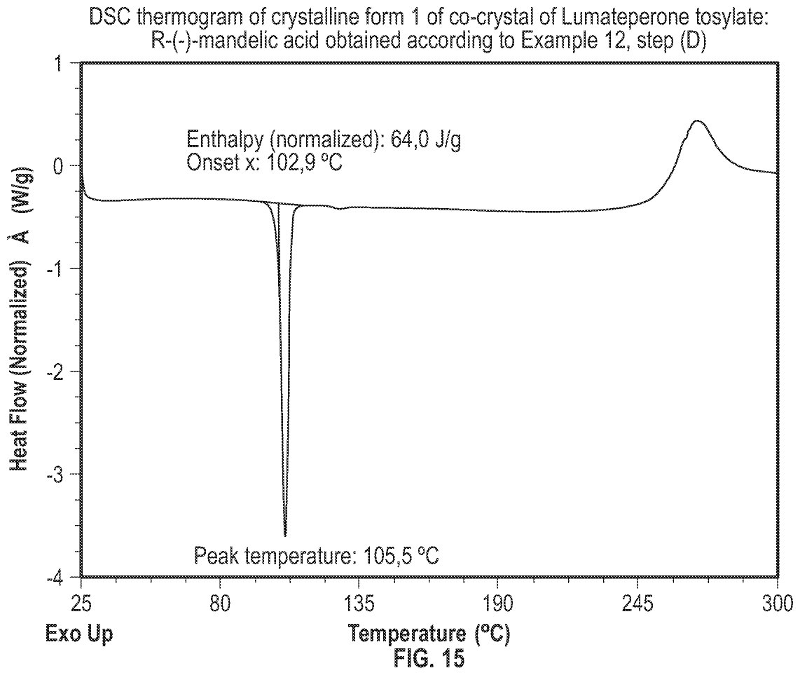

FIG. 16 shows TGA thermogram of crystalline form 1 of co-crystal of Lumateperone tosylate:R-(-)-mandelic acid obtained according to Example 12, step (D).

FIG. 17 shows 13C solid state NMR spectrum of crystalline form 1 of co-crystal of Lumateperone tosylate:R-(-)-mandelic acid, step (D).

FIG. 18 shows FTIR spectrum of crystalline form 1 of co-crystal of Lumateperone tosylate:R-(-)-mandelic acid obtained according to Example 12, step (D).

FIG. 19 shows an X-ray powder diffractogram (XRPD) of crystalline form A of Lumateperone dibenzenesulfonate obtained according to Example 13, step (C).

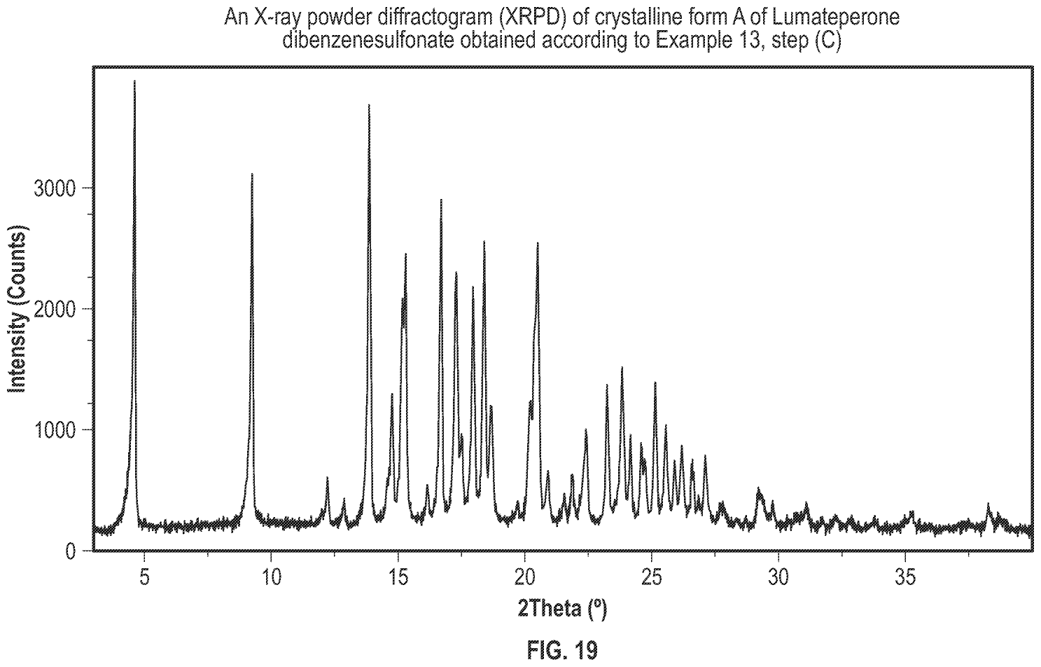

FIG. 20 shows DSC thermogram of crystalline form A of Lumateperone dibenzenesulfonate obtained according to Example 13, step (C).

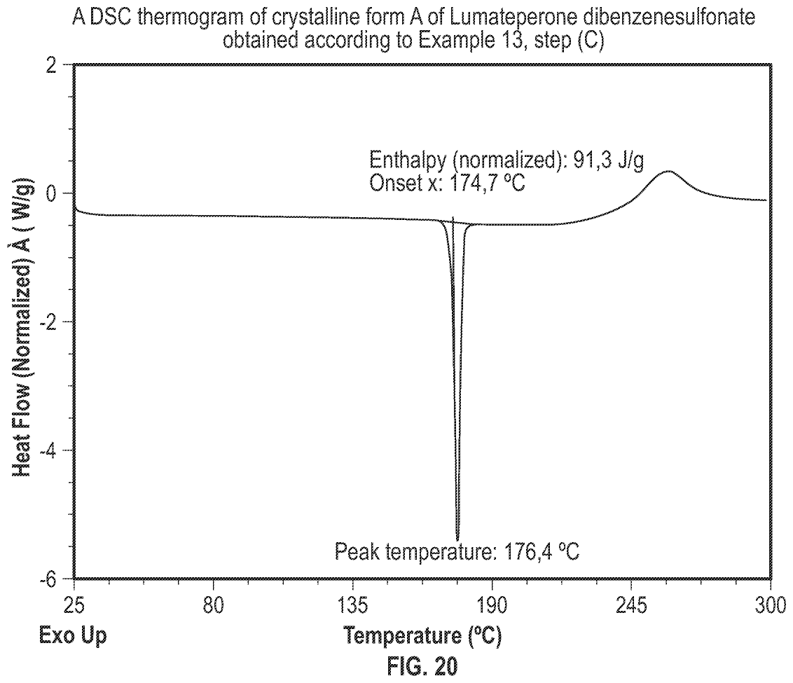

FIG. 21 shows TGA thermogram of crystalline form A of Lumateperone dibenzenesulfonate obtained according to Example 13, step (C).

FIG. 22 shows FTIR spectrum of crystalline form A of Lumateperone dibenzenesulfonate obtained according to Example 13, step (C).

FIG. 23 shows an X-ray powder diffractogram (XRPD) of crystalline form 1 of co-crystal of Lumateperone tosylate:R-(-)-mandelic acid obtained according to Example 13, step (E).

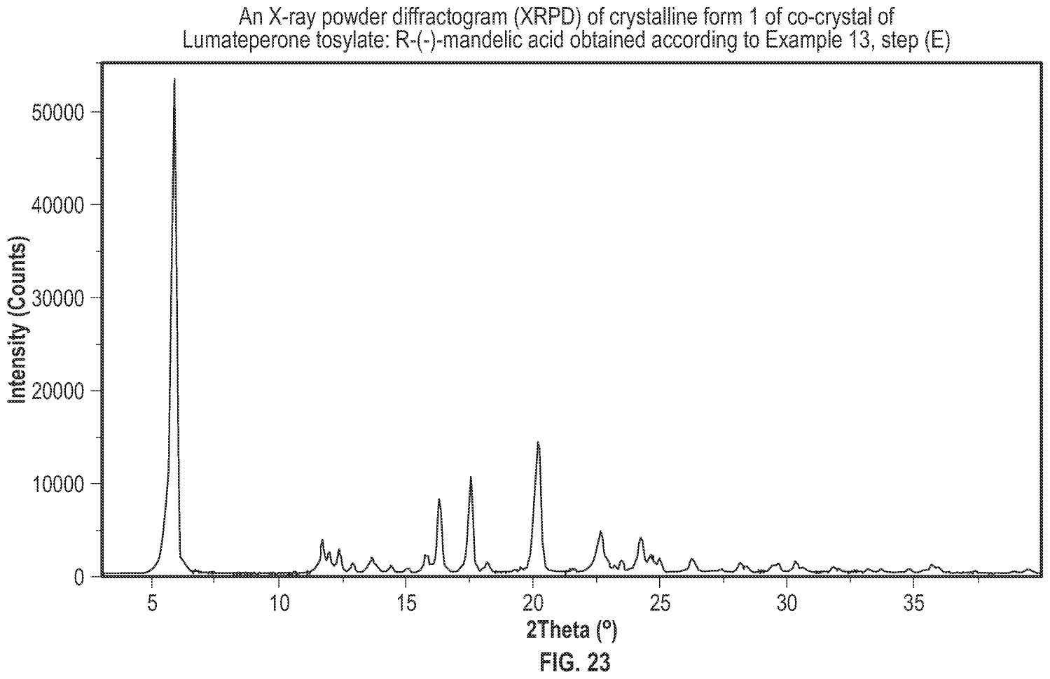

FIG. 24 shows DSC thermogram of crystalline form 1 of co-crystal of Lumateperone tosylate:R-(-)-mandelic acid obtained according to Example 13, step (E).

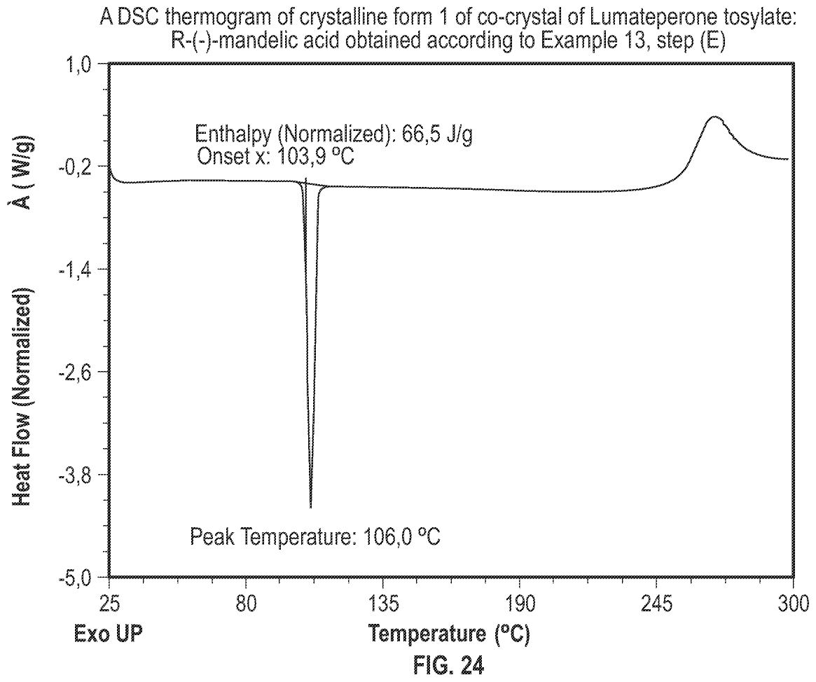

FIG. 25 shows TGA thermogram of crystalline form 1 of co-crystal of Lumateperone tosylate:R-(-)-mandelic acid obtained according to Example 13, step (E).

FIG. 26 shows FTIR spectrum of crystalline form 1 of co-crystal of Lumateperone tosylate:R-(-)-mandelic acid obtained according to Example 13, step (E).

FIG. 27 shows crystal structure of the co-crystal of Lumateperone tosylate:R-(-)-mandelic acid with labelled heteroatoms.

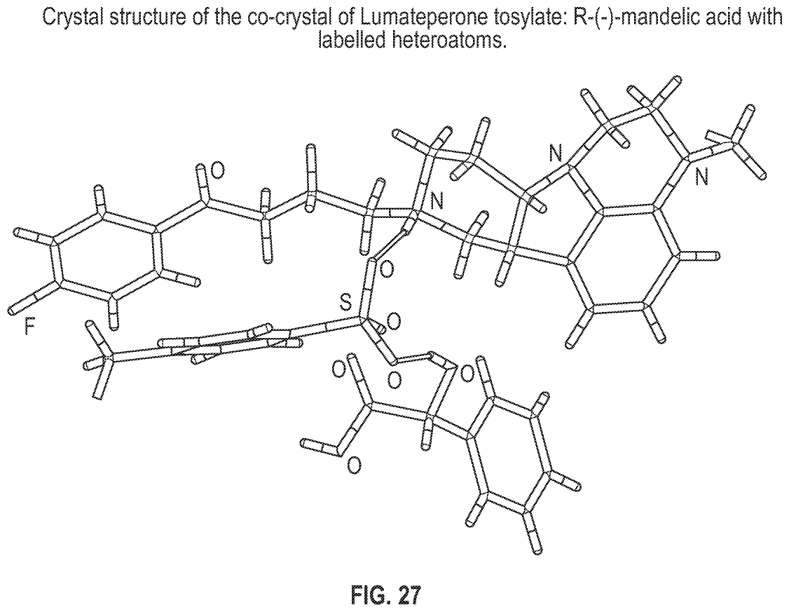

FIG. 28 shows the calculated powder diffraction pattern of the co-crystal of Lumateperone tosylate:R-(-)-mandelic acid compared to the bulk material obtained according to example 13, step (E).

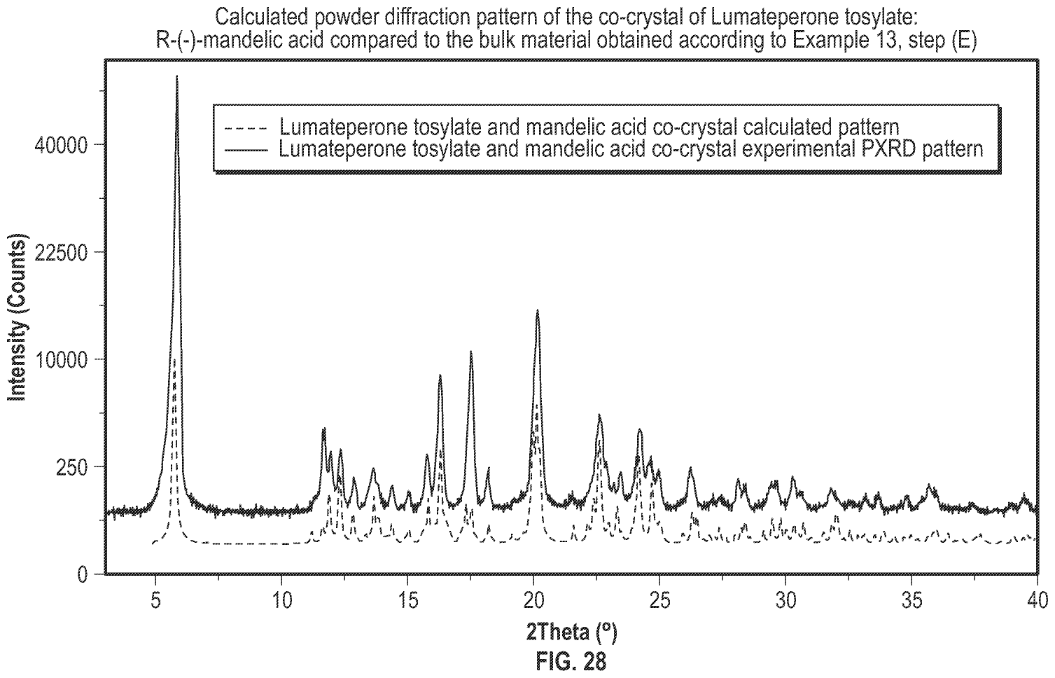

### DETAILED DESCRIPTION OF THE DISCLOSURE

The present disclosure relates to improved solid state forms of Lumateperone Besylate. The present disclosure also relates to processes for preparation thereof, and pharmaceutical compositions including the disclosed solid state forms.

The solid state form of Lumateperone Besylate according to the present disclosure may have advantageous properties selected from at least one of: chemical or polymorphic purity, flowability, solubility, dissolution rate, bioavailability, morphology or crystal habit, stability - such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

The present disclosure further relates to crystalline Lumateperone tosylate:R-(-)-mandelic acid, a solid state form thereof, processes for preparation thereof, and pharmaceutical compositions including the crystalline Lumateperone tosylate:R-(-)-mandelic acid or of the described solid state form thereof.

The disclosure also relates to conversion of the crystalline Lumateperone tosylate:R-(-)-mandelic acid or the described solid state form thereof to other forms thereof, such as Lumateperone tosylate or other salts thereof and solid state forms thereof.

Crystalline Lumateperone tosylate:R-(-)-mandelic acid according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form such as Crystalline Lumateperone tosylate:R-(-)-mandelic acid or Lumateperone besylate may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Lumateperone Besylate or Lumateperone tosylate:R-(-)-mandelic acid, referred to herein as being characterized by graphical data “as depicted in” a Figure will thus be understood to include any crystal forms of the Lumateperone Besylate or Lumateperone tosylate:R-(-)-mandelic acid, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, the solid state form of Lumateperone Besylate or Lumateperone tosylate:R-(-)-mandelic acid described herein as is substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Lumateperone Besylate or Lumateperone tosylate:R-(-)-mandelic acid respectively. Accordingly, in some embodiments of the disclosure, the described solid state form of Lumateperone Besylate may contain from about 1% to about 20% (w/w), from about 5% to about 20% (w/w), or from about 5% to about 10% (w/w) of one or more other solid state forms of the Lumateperone Besylate. In some embodiments of the disclosure, the described solid state form of Lumateperone tosylate:R-(-)-mandelic acid may contain from about 1% to about 20% (w/w), from about 5% to about 20% (w/w), or from about 5% to about 10% (w/w) of one or more other solid state forms of the Lumateperone tosylate:R-(-)-mandelic acid.

As used herein, unless stated otherwise, XRPD peaks reported herein are optionally measured using Cuk α radiation, λ=1.54184 Å, at a temperature of 25±3° C.

As used herein, DSC measurements are obtained at a heating rate of 10° C./minute, under a nitrogen flow of 50 mL/min.

As used herein, TGA measurement are obtained at a heating rate of 10° C./minute, under

a nitrogen flow of 30 mL/min.

As used herein, and unless stated otherwise, the term “anhydrous” in relation to crystalline Lumateperone Besylate or Lumateperone tosylate:R-(-)-mandelic acid relates to crystalline Lumateperone Besylate or Lumateperone tosylate:R-(-)-mandelic acid which does not include any crystalline water (or other solvents) in a defined, stoichiometric amount within the crystal. Moreover, an “anhydrous” form does not contain more than about 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein crystalline form A of Lumateperone Tosylate refers to a

crystalline form which may be characterized by X-ray powder diffraction pattern as disclosed in U.S. Pat. No. 8,648,077.

As used herein, the term “isolated” in reference to solid state form of Lumateperone Besylate of the present disclosure corresponds to solid state form of Lumateperone Besylate that is physically separated from the reaction mixture in which it is formed.

As used herein, the term “isolated” in reference to crystalline Lumateperone tosylate:R-(-)-mandelic acid of the present disclosure corresponds to crystalline Lumateperone tosylate:R-(-)-mandelic acid that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

As used herein, the term “reduced pressure” refers to a pressure of from about 10 mbar to about 500 mbar, or about 50 mbar.

As used herein and unless indicated otherwise, the term “ambient conditions” refer to atmospheric pressure, 22-24° C.

As used herein, and unless indicated otherwise, the term “chlorinated solvent” refers to a C1-C6 chlorinated hydrocarbon. In some embodiments, the chlorinated solvents are selected from carbon tetrachloride, dichloromethane (CH₂Cl₂), dichloroethane, chlorobenzene, and chloroform.

As used herein, and unless indicated otherwise, the term “one pot process” refers to a continuous process for preparing a desired product, in which penultimate product is converted to the desired product in the same vessel.

As used herein, and unless indicated otherwise, the term “Protecting group” refers to a grouping of atoms that, when attached to a reactive functional group in molecule masks, reduces or prevents reactivity of the functional group. Examples of protecting groups can be found in Greene and Wuts “Greene's Protective Groups in Organic Synthesis”, 4th Edition, publ. Wiley, 2006 and Harrison et al., “Compendium of Synthetic Organic Methods”, Vols. 1-8 (John Wiley and Sons, 1971-1996).

Representative amine protecting groups include, but are not limited to, Fmoc, cbz, benzyl, trityl, Boc, trifluoroacetyl derivative, phthalic anhydride, or succinic anhydride derivatives.

As used herein, “halogen” or “halide” refers to fluoride, chloride, bromide or iodide. In certain embodiments, the halogen is bromide or iodide.

The present disclosure relates to Besylate salt of Lumateperone having the formula:

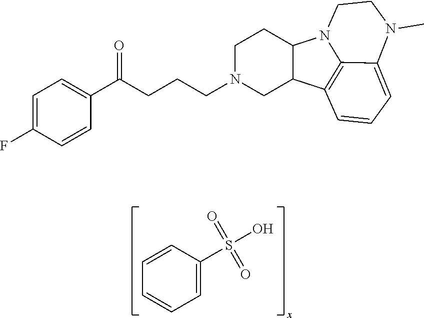

wherein X can be any number between 0.5 and 3.

In some embodiments X may be 0.5, 1, 1.5, 2, 2.5 or 3. Preferably, X may be 1 or 2, and more preferably X is 2.

In embodiments, the besylate salt of Lumateperone tesylate, according to the above embodiments is crystalline.

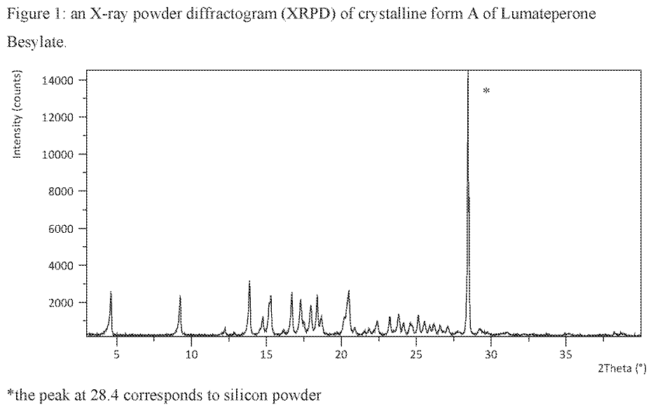

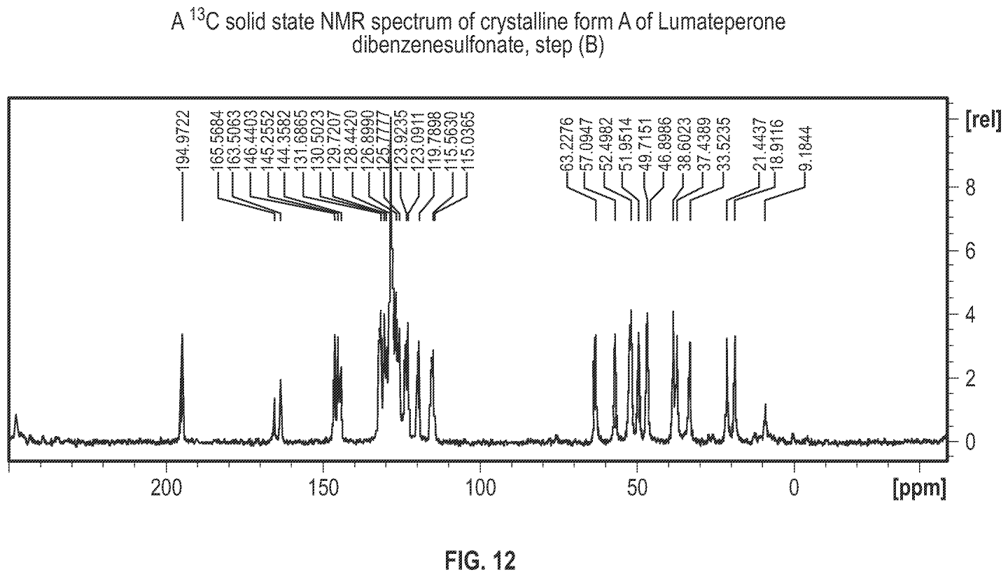

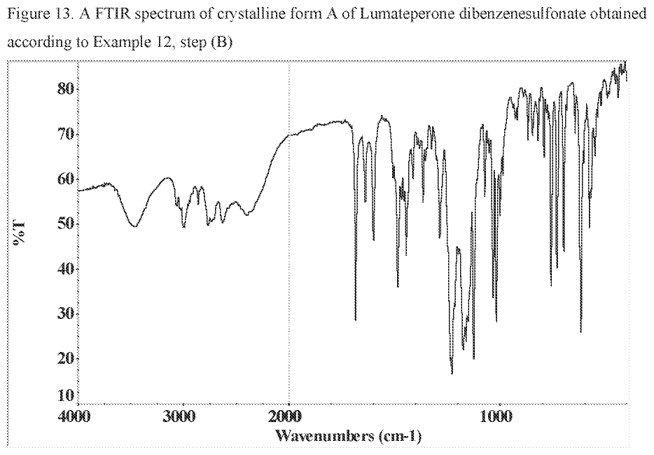

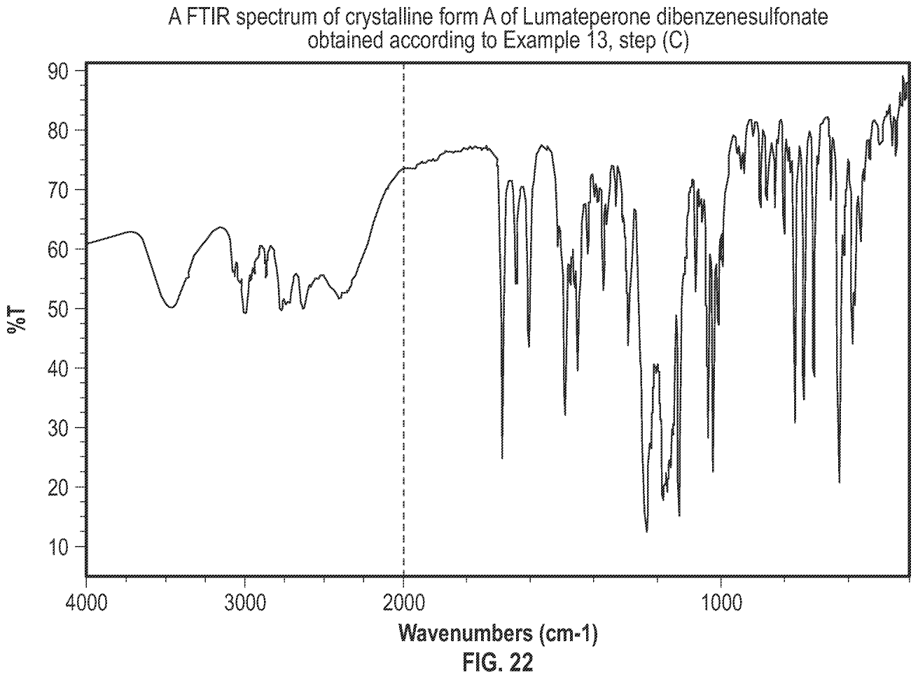

The present disclosure further relates to a crystalline form of Lumateperone Besylate according to any one of the above embodiments, designated form A. The crystalline form A of Lumateperone Besylate may be characterized by data selected from one or more of the following: an XRPD pattern having peaks at 4.6, 9.2, 13.9, 20.5 and 23.2 degrees 2-theta±0.2 degrees 2-theta; an XRPD pattern substantially as depicted in FIG. 1, 9 or 19; a solid state ¹³C NMR spectrum substantially as depicted in FIG. 12; a solid state ¹³C NMR spectrum having peaks at 195.0, 163.5, 119.8 and 57.1 ppm±0.2 ppm; a solid state ¹³C NMR spectrum having the following chemical shift absolute differences between said characteristic peaks at 195.0, 163.5, 119.8 and 57.1 ppm±0.2 ppm and a reference peak at 33.5 ppm±0.2 ppm of 161.5, 130.0, 86.3 and 23.6±0.1 ppm; an FT-IR spectrum substantially as depicted in FIG. 13 or FIG. 22; an FT-IR spectrum having absorptions at 2392, 1638, 1483, 1226, 1122 and 613 cm⁻¹±4 cm⁻¹; and combinations of these data.

Crystalline Form A of Lumateperone Besylate may be further characterized by an XRPD pattern having peaks at 4.6, 9.2, 13.9, 20.5 and 23.2 degrees 2-theta±0.2 degrees 2-theta, and also having one, two, three, four or five additional peaks selected from 15.3, 16.7, 18.0, 22.4 and 25.1 degrees two theta±0.2 degrees two theta.

In embodiments, crystalline form A is a dibesylate salt of Lumateperone, more preferably a dibesylate salt of lumateperone.

In some embodiments crystalline form A of Lumateperone Besylate may be isolated.

In some embodiments crystalline form A of Lumataperone Besylate may be anhydrous.

In some embodiments the disclosure relates to processes for preparation of crystalline form A of Lumateperone Besylate.

The process for the preparation of crystalline Lumateperone Besylate, in embodiments crystalline form A of Lumateperone Besylate, may comprise crystallisation of Lumateperone Besylate from a solvent comprising one or more polar solvents. In embodiments, the solvent may be a polar aprotic solvent optionally in combination with a polar protic solvent. Suitable polar aprotic solvents include nitriles, ethers, ketones, and esters, or mixtures thereof. In any embodiment of this process or in any embodiment or aspect involving this process step, the polar aprotic solvent may be selected from: acetonitrile, methyl ethyl ketone, acetone, methyl t-butyl ether and isopropyl acetate. In any embodiment of this process or in any embodiment or aspect involving this process step, the polar aprotic solvent is a ketone, and more preferably acetone. Suitable polar protic solvents include alcohols, especially isopropanol or ethanol, and particularly isopropanol.

In embodiments, crystalline Form A of Lumateperone Besylate may be prepared by crystallisation of Lumateperone Besylate from a solvent mixture comprising acetone and isopropanol. The volume ratio of the polar aprotic solvent to polar protic solvent, such as acetone:isopropanol, may be from about 1:10 to about 10:1, about 2:1 to about 8:1, about 3:1 to about 8:1, about 5:1 to about 8:1, about 5:1 to about 7:1, or about 6:1. The crystallisation may be carried out by cooling a solution of Lumateperone besylate in the solvent or solvent mixture. In any embodiment of this process or in any embodiment or aspect involving this process step, the solution is cooled to a temperature of 0° C. to about 15° C., particularly 0° C. to about 10° C. or about 5° C. The solution may preferably be cooled from an elevated temperature of about 30° C. to about 70° C., about 40° C. to about 60° C., or about 50° C. The hot mixture may be stirred for about 5 minutes to about 120 minutes, about 15 minutes to about 60 minutes or about 15 to about 45 minutes, or about 30 minutes. The hot solution may first be allowed to cool to room temperature, and then further cooled to the temperature ranges below room temperature as indicated. The cooling to below room temperature may be carried out over about 30 minutes to about 240 minutes, about 60 minutes to about 180 minutes, or about 80 minutes to about 140 minutes, or about 120 minutes. The cooled mixture may be further stirred for about 5 minutes to about 120 minutes, about 15 minutes to about 60 minutes or about 15 to about 45 minutes, or about 30 minutes. The product may be isolated from the mixture by filtration.

In one embodiment the disclosure relates to a process for preparation of crystalline Lumateperone Besylate, such as crystalline form A of Lumatepereone Besylate, wherein the process includes: a) providing Lumateperone optionally in the form of a solution in one or more polar solvents; b) adding benzenesulfonic acid, optionally in the form of a solution in one or more polar solvents; c) optionally stirring; d) optionally cooling; and c) optionally isolating crystalline Lumateperone Besylate.

The process may include additional washing and drying steps.

In embodiments step a) includes providing Lumateperone in a polar aprotic solvent, preferably a ketone, more preferably acetone. In embodiments, step a) includes providing a solution of Lumateperone in acetone at a temperature of about 40° C. to about 60° C.

In embodiments step b) includes adding a solution of benzenesulfonic acid in one or more polar solvent, in embodiments in a mixture of a polar aprotic solvent and a polar protic solvent, in some embodiments a mixture of acetone and isopropanol.

In embodiments, the polar solvents in step a) and/or step b) include nitriles, ethers, ketones, alcohols and esters or mixtures thereof. In embodiments, the solvents are selected from acetonitrile, methyl ethyl ketone, acetone, isopropanol, methyl t-butyl ether and isopropyl acetate and ethanol. In embodiments, the polar aprotic solvent is acetone and the polar protic solvent is isopropanol.

In embodiments step c) includes stirring the reaction mixture at a temperature of about 40° C. to about 60° C.

In embodiments step d) includes cooling the reaction mixture to room temperature and stirring and optionally further cooling to a temperature of about 0° C. to about 15° C. and stirring.

In another embodiment the disclosure relates to a process for preparation of crystalline dibenzenesulfonate salt of Lumateperone, preferably crystalline form A of dibenzenesulfonate salt of Lumateperone, wherein the process may comprise crystallisation of Lumateperone dibesylate from a solvent comprising one or more polar solvents. Particularly, the solvent may be at least one, or the solvent may comprise at least two, polar solvents. In any embodiment of this process or in any embodiment or aspect involving this process step, the solvent may comprise at least two polar aprotic solvents. Suitable polar aprotic solvents include nitriles, ethers, ketones, and esters or mixtures thereof. In any embodiment of this process or in any embodiment or aspect involving this process step, the solvent is a mixture of a nitrile and a ketone. In embodiments, crystalline Form A of Lumateperone dibesylate may be prepared by crystallisation of Lumateperone dibesylate from a solvent mixture comprising acetonitrile and methylethylketone. The volume ratio of the nitrile to ketone, such as acetonitrile : methylethylketone, can be from about 6:1 to about 1:4, or about 5:1 to about 1:3; or about 4:1 to about 1:3. The crystallisation may be effected by cooling a hot (preferably 40° C. to about 70° C., about 45° C. to about 60° C., or about 50° C.) mixture of the lumateperone dibesylate In any embodiment of this process or in any embodiment or aspect involving this process step, the cooling may be to about 10° C.to about 30° C., about 15° C. to about 25° C., or about 20° C. The hot mixture may be stirred for about 5 minutes to about 120 minutes, about 15 minutes to about 60 minutes or about 15 to about 45 minutes, preferably about 30 minutes. The cooled mixture may be further stirred for about 30 minutes to about 24 hours, about 1 hour to about 12 hours, or about 1 hour to about 8 hours. The crystallisation may be conducted in the presence of seed crystals of Lumateperone Dibesylate. Thus, seed crystals can be added to the hot mixture prior to cooling. However, seeding is optional.

In another embodiment the disclosure relates to a process for preparation of crystalline dibenzenesulfonate salt of Lumateperone, preferably crystalline form A of dibenzenesulfonate salt of Lumateperone, wherein the process comprises:
a) Providing lumateperone in acetonitrile at a temperature of about 40° C. to about 60° C.;
b) Adding benzenesulfonic acid;
c) Adding methyl ethyl ketone to obtain a suspension;
d) Optionally stirring the suspension at a temperature of about 40° C. to about 60° C.;
c) Optionally cooling to room temperature and stirring; and
f) optionally isolating crystalline dibenzenesulfonate salt of Lumateperone

In yet another embodiment the disclosure relates to a process for preparation of crystalline dibenzenesulfonate salt of Lumateperone, preferably crystalline form A of dibenzenesulfonate salt of Lumateperone, wherein the process comprises:
a) Providing lumateperone in a mixture of acetonitrile and ethyl methyl ketone at a temperature of about 40° C. to about 60° C.;
b) Adding benzenesulfonic acid, in the form of a solution in acetonitrile;
c) Optionally seeding with Lumateperone dibenzenesulfonate seeds
d) Optionally stirring at a temperature of about 40° C. to about 60° C.;
g) Optionally cooling to room temperature and stirring;
e) Adding methyl ethyl ketone and stirring; and
f) Optionally isolating crystalline dibenzenesulfonate salt of Lumateperone.

The amount of benzenesulfonic acid employed in the process for preparing Lumateperone dibesylate according to any aspect or embodiment of the present invention can be from about 1 to about 3 mol equivalent, about 1.2 to about 2.5 equivalent, or about 1.3 to about 2.3 equivalent. Preferably about 2 to about 2.6 mol equivalent, or about 2.1 to about 2.3 mol equivalent of benzenesulfonic acid is employed.

The present disclosure also provides uses of the said solid state forms of Lumateperone Besylate (such as Lumateperone dibesylate) in the preparation of other solid state forms of Lumateperone Besylate, Lumateperone or other salts of Lumateperone and solid state forms thereof.

The present disclosure also provides the said solid state forms of Lumateperone Besylate (such as Lumateperone dibesylate) for use in the preparation of other solid state forms of Lumateperone Besylate, Lumateperone or other salts of Lumateperone and solid state forms thereof.

The present invention encompasses a process for preparing other Lumateperone salts or solid state forms of Lumateperone. The process includes preparing a solid state form of Lumateperone Besylate (such as Lumateperone dibesylate) by the processes of the present invention, and converting that salt to another Lumateperone salt. The conversion can be done, for example, by a process including basifying the above described Lumateperone salt and/or solid state form thereof and reacting the obtained Lumateperone base with an appropriate acid, to obtain the corresponding salt. Alternatively, the conversion can be done by salt switching, i.e., reacting Lumateperone Besylate (such as Lumateperone dibesylate) or a solid state form thereof, with an acid having a pKₐ which is lower than the pKₐ of the acid of Lumateperone Besylate. Advantageously, Lumateperone dibesylate offers significant impurity purging capabilities and enables preparation of Lumateperone or salts thereof in high purity and without the use of chromatographic methods.

In a further aspect, the present invention provides the use of the benzenesulfonate salt of Lumateperone as described in any embodiment herein, in a process for purifying Lumateperone. The present invention thus provides a process for purifying Lumateperone, wherein the process may comprise; a) providing a solution of Lumateperone and benzenesulfonic acid; b) crystallizing the Lumateperone dibenzenesulfonate; and c) converting the Lumateperone dibenzenesulfonate to form purified Lumateperone. In any embodiment of this process, steps (a) and (b) may be carried out according to a process as defined in any of the above-described processes for preparing Lumateperone dibesylate. The process may further comprise converting the purified Lumateperone to a Lumateperone salt, such as Lumateperone tosylate, or Lumateperone tosylate:R-(-)-mandelic acid. Lumateperone tosylate:R-(-)-mandelic acid obtainable by this process advantageously has high purity. In any aspect or embodiment of the present invention, the Lumateperone tosylate:R-(-)-mandelic acid has a total impurity content of not more than 0.3% area percent, or not more than 0.2% area percent, or not more than 0.1% arca percent, particularly not more than 0.08% area percent, as measured by UPLC.

In another embodiment, the present disclosure encompasses use of the described solid state forms of Lumateperone Besylate (in embodiments Lumateperone dibesylate) in the preparation of pharmaceutical compositions and/or formulations, optionally for the treatment of treatment of central nervous system disorders such as schizophrenia, bipolar disorder, depression, sleep and behavioral disturbance in dementia, autism and other neuropsychiatric disorders.

In another embodiment, the present disclosure encompasses the described solid state forms of Lumateperone Besylate (in embodiments Lumateperone dibesylate) for use in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

The present disclosure further provides pharmaceutical compositions including the solid state forms of Lumateperone Besylate (in embodiments Lumateperone dibesylate) according to the present disclosure.

In yet another embodiment, the present disclosure encompasses pharmaceutical formulations including the described solid state forms of Lumateperone Besylate (in embodiments Lumateperone dibesylate) and at least one pharmaceutically acceptable excipient.

The present disclosure encompasses processes to prepare said pharmaceutical formulations of Lumateperone Besylate (in embodiments Lumateperone dibesylate) including combining the described solid state forms of Lumateperone Besylate (in embodiments Lumateperone dibesylate) and at least one pharmaceutically acceptable excipient.

The solid state forms defined herein as well as the pharmaceutical compositions or formulations of the improved solid state form of Lumateperone Besylate (in embodiments Lumateperone dibesylate) can be used as medicaments, particularly for the treatment of central nervous system disorders, as specified above.

The present disclosure also provides methods of treating, particularly for the treatment of central nervous system disorders, as specified above including administering a therapeutically effective amount of the improved solid state form of Lumateperone Besylate (in embodiments Lumateperone dibesylate) of the present disclosure, or at least one of the herein described pharmaceutical compositions or formulations, to a subject suffering from the above specified diseases, or otherwise in need of the treatment.

The present disclosure also provides uses of the improved solid state form of Lumateperone Besylate (in embodiments Lumateperone dibesylate) of the present disclosure, or at least one of the above pharmaceutical compositions or formulations for the manufacture of medicaments, particularly for medicaments, particularly for the treatment of central nervous system disorders, as specified above.

The present invention further provides crystalline Lumateperone tosylate:R-(-)-mandelic acid.

As used herein, crystalline Lumateperone tosylate:R-(-)-mandelic acid is a distinct molecular species. In one embodiment crystalline Lumateperone tosylate:R-(-)-mandelic acid may be a co-crystal of lumateperone tosylate and R-(-)-mandelic acid.

As used herein, a co-crystal is a crystalline material composed of two or more molecules within the same crystal lattice, wherein the molecules interact with each other via non-ionic interactions.

In embodiments the molar ratio between the active pharmaceutical ingredient (lumateperone tosylate) and the conformer (R-(-)-mandelic acid) is between 1:1.5 and 1.5:1, in some embodiments between 1:1.25 and 1.25:1, in other embodiments about 1:1.

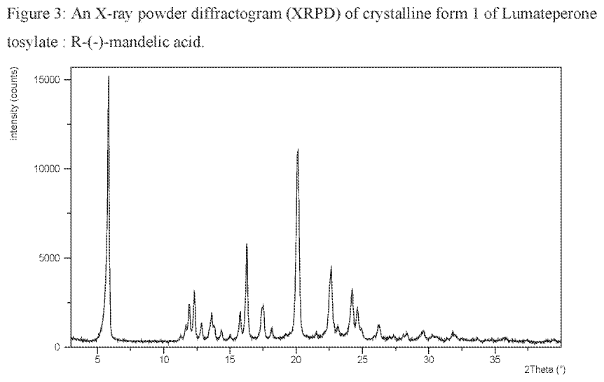

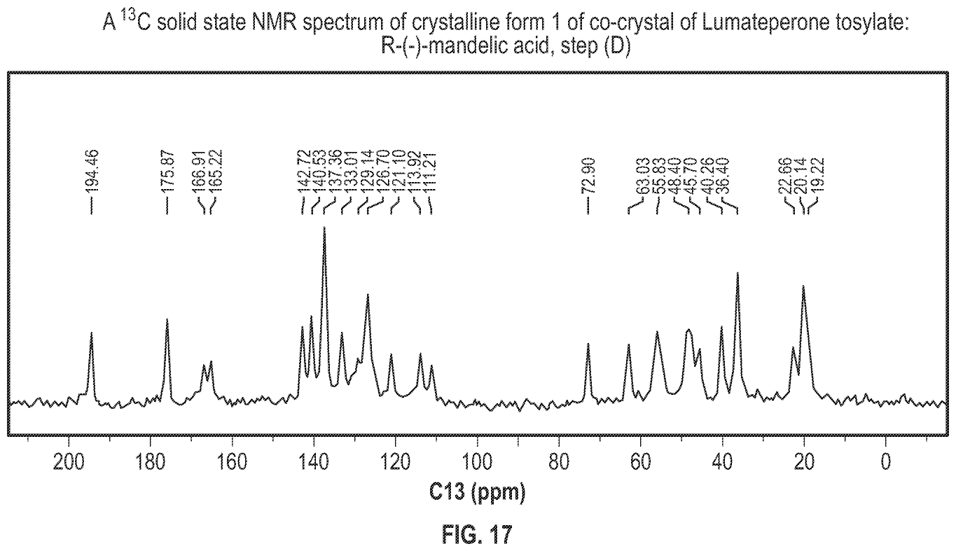

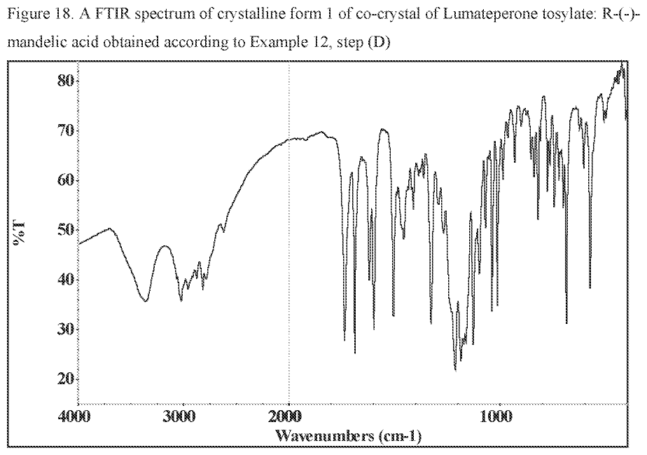

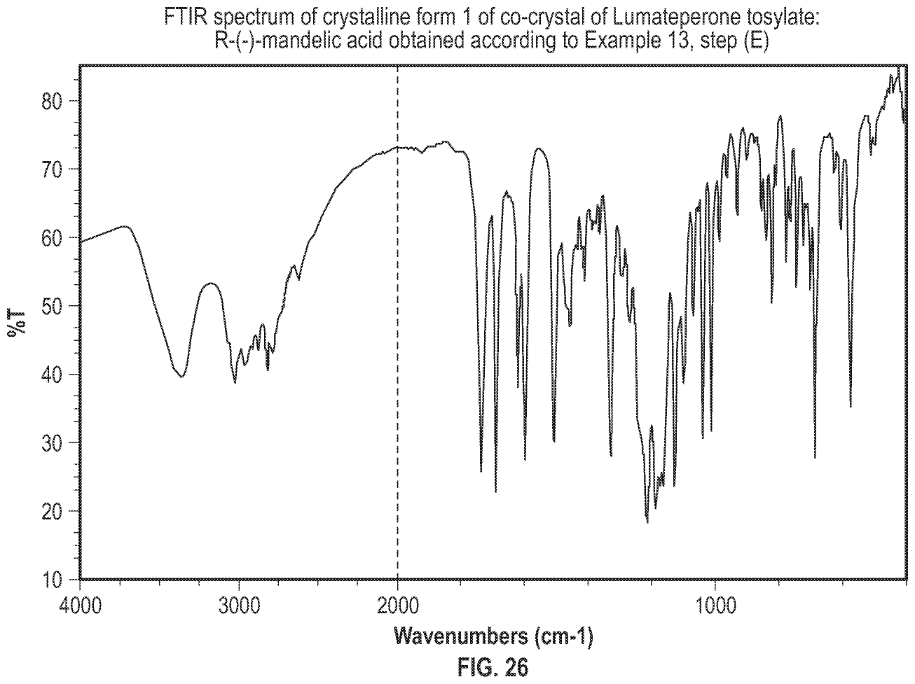

The present disclosure further relates to a crystalline form of Lumateperone tosylate:R-(-)-mandelic acid designated form 1. The crystalline form 1 of Lumateperone tosylate:R-(-)-mandelic acid may be characterized by data selected from one or more of the following: an XRPD pattern having peaks at 5.8, 12.3, 16.3, 20.1 and 22.7 degrees 2-theta±0.2 degrees 2-theta; an XRPD pattern substantially as depicted in FIG. 3, 14 or 23; a solid state ¹³C NMR spectrum substantially as depicted in FIG. 17; a solid state ¹³C NMR spectrum having peaks at 175.9, 133.0, 72.9 and 36.4 ppm±0.2 ppm; a solid state ¹³C NMR spectrum having the following chemical shift absolute differences between said characteristic peaks at 175.9, 133.0, 72.9 and 36.4 ppm±0.2 ppm and a reference peak at 20.1 ppm±0.2 ppm of 155.8, 112.9, 52.8 and 16.3±0.1 ppm; an FT-IR spectrum substantially as depicted in FIG. 18 or FIG. 26; an FT-IR spectrum having absorptions at 1736, 1184, 1067 and 697 cm⁻¹±4 cm⁻¹; and combinations of these data.

Alternatively, form 1 of Lumateperone tosylate:R-(-)-mandelic acid can be characterized by the following unit cell data:

| cell_length_a | 7.65 | Å |
|---|---|---|
| cell_length_b | 8.39 | Å |
| cell_length_c | 15.53 | Å |
| cell_angle_alpha | 78.75° | |
| cell_angle_beta | 78.44° | |
| cell_angle_gamma | 70.22° | |
| cell_volume | 911 | Å³ |
| cell_measurement_temperature, 293 K | | |
| symmetry_space_group_P -1 | | |

The crystalline form 1 of Lumateperone tosylate:R-(-)-mandelic acid can be characterized by any combination of the above data.

Crystalline Form 1 of Lumateperone tosylate:R-(-)-mandelic acid may be further characterized by an XRPD pattern having peaks at 5.8, 12.3, 16.3, 20.1 and 22.7 degrees 2-theta±0.2 degrees 2-theta, and also having one, two, three, four or five additional peaks selected from 12.9, 13.6, 15.8, 24.2 and 24.6 degrees two theta±0.2 degrees two theta.

In one embodiment crystalline form 1 of Lumateperone tosylate:R-(-)-mandelic acid may be may be a co-crystal.

In some embodiments crystalline form 1 of Lumateperone tosylate:R-(-)-mandelic acid may be anhydrous.

In some embodiments crystalline form 1 of Lumateperone tosylate:R-(-)-mandelic acid may be isolated.

In some embodiments the disclosure relates to processes for preparation of crystalline Lumateperone tosylate:R-(-)-mandelic acid.

In an embodiment, the present invention relates to a process for the preparation of a crystalline form of Lumateperone tosylate:R-(-)-mandelic acid, in embodiments crystalline form 1 of Lumateperone tosylate:R-(-)-mandelic acid, comprising suspending Lumateperone tosylate with R-(-)-mandelic acid in one or more organic solvents. The solvent may be an aromatic hydrocarbon, optionally in combination with an ester. In any embodiment of this process or in any embodiment or aspect involving this process step, the aromatic hydrocarbon may be toluene. In any embodiment of this process or in any embodiment or aspect involving this process step, the ester may be isopropylacetate. Accordingly, in any embodiment of this process, or any embodiment or aspect involving this process step, the process comprises suspending Lumateperone tosylate with R-(-)-mandelic acid in a mixture comprising toluene and isopropyl acetate. In any embodiment of this process or in any embodiment or aspect involving this process step, the volume ratio of toluene and isopropylacetate is from about 9:1 to about 5:4, from about 4:1 to about 5:3, or about 4:3. In any embodiment of this process or in any embodiment or aspect involving this process step, about 1 to about 1.5 mol equivalent, or about 1.1 to about 1.6 mol equivalent, or about 1.1 to about 1.5 mol equivalent of R-(-)-mandelic acid to Lumateperone tosylate is employed

In any embodiment of this process or in any embodiment or aspect involving this process step, the process may comprise cooling a mixture of Lumateperone tosylate and R(-)-mandelic acid in the solvent or combination of solvents. The mixture may be cooled from a temperature of about 40° C. to about 80° C., about 45° C. to about 75° C., or about 50° C. to about 70° C. In any embodiment of this process or in any embodiment or aspect involving this process step , the mixture may be cooled to temperature of about −10° C. to about 15° C., or about −5° C. to about 10° C. or about 0° C.to about 5° C.

The process may alternatively comprise stirring a mixture of Lumateperone tosylate and R(-)-mandelic acid in the solvent or combination of solvents at a temperature of about 10° C. to about 30° C., about 15° C. to about 25° C., or about 20° C. In any embodiment of this process or in any embodiment or aspect involving this process step, the process may comprise stirring a mixture of Lumateperone tosylate and R(-)-mandelic acid in the solvent or combination of solvents at a temperature of about 10° C. to about 30° C., about 15° C. to about 25° C., or about 20° C.

In any embodiment of this process or in any embodiment or aspect involving this process step, the process is carried out under an inert atmosphere, preferably under nitrogen or argon.

In embodiments the disclosure relates to a process for preparation of crystalline Lumateperone tosylate:R-(-)-mandelic acid, such as crystalline form 1 of Lumateperone tosylate:R-(-)-mandelic acid, wherein the process includes: a) providing Lumateperone tosylate and R-(-)-mandelic acid optionally in the form of a suspension in one or more organic solvents; b) optionally stirring; c) optionally cooling; and d) optionally isolating crystalline R-(-)-mandelic acid.

The process may include additional washing and drying steps.

In embodiments the Lumateperone tosylate is provided in amorphous form.

In other embodiments, Lumateperone tosylate and R-(-)-mandelic acid are provided in an aromatic solvent, such as toluene.

In an embodiment, the disclosure relates to a process for the preparation of a crystalline form of Lumateperone tosylate:R-(-)-mandelic acid, in embodiments crystalline form 1 of Lumateperone tosylate:R-(-)-mandelic acid, comprising suspending Lumateperone tosylate with R-(-)-mandelic acid in one or more organic solvents. In any embodiment of this process or in any embodiment or aspect involving this process step, the solvent can be an aromatic hydrocarbon, optionally with an ester. In any embodiment of this process or in any embodiment or aspect involving this process step, the solvent may be toluene optionally in combination with isopropylacetate.

Preferably about 1 to about 1.5 mol equivalent, or about 1.1 to about 1.6 mol equivalent, or about 1.1 to about 1.5 mol equivalent of R-(-)-mandelic acid to Lumateperone tosylate is employed.

The preparation of Lumateperone tosylate:R-(-)-mandelic acid may comprise cooling a hot (preferably 40° C. to about 80° C., about 45° C. to about 75° C., or about 50° C. to about 70° C.) mixture of Lumateperone tosylate and R(-)-mandelic acid. Preferably the cooling may be to a temperature of −10° C. to about 15° C., particularly −5° C. to about 10° C. or about 0° C. to about 5° C. Alternatively the mixture of Lumateperone tosylate and R(-)-mandelic acid may be stirred in the solvent at a temperature of about 10° C. to about 30° C., about 15° C. to about 25° C., or about 20° C.

The process for the preparation of Lumateperone tosylate:R-(-)-mandelic acid according to any aspect or embodiment of the present invention is preferably carried out in an inert atmosphere, e.g. under nitrogen, or preferably under argon.

In another embodiment the disclosure relates to a process for preparation of crystalline form of Lumateperone tosylate:R-(-)-mandelic acid, preferably crystalline form 1 of Lumateperone tosylate:R-(-)-mandelic acid, wherein the process comprises:
a) Providing lumateperone in a one or more organic solvents;
b) Adding p-toluene sulfonic acid and R-(-)-mandelic acid;
c) Optionally stirring at a temperature of about 60° C. to about 80° C.;
d) Colling down to a temperature of about 0° C. to about 10° C.;
e) Optionally stirring at a temperature of about 0° C. to about 10° C.; and
f) Optionally isolating crystalline Lumateperone tosylate:R-(-)-mandelic acid

In embodiments, in step a) lumateperone is provided in a solvent system comprising toluene.

In embodiments, in step a lumateperone is provided in a mixture of toluene and i-propylacetate.

In embodiments, ratio of toluene and i-propylacetate in step a may be from about 9:1 to about 5:4, preferably from about 4:1 to about 5:3, more preferably about 4:3/In embodiments, steps a, b, c, d and/or e may advantageously be performed in an inert atmosphere. In any embodiment of this process or in any embodiment or aspect involving this process step, steps a, b, c, d, e and/or f, are carried out under a nitrogen or argon(in embodiments argon) atmosphere.

In a specific embodiment, the disclosure relates to a process for preparation of crystalline form of Lumateperone tosylate:R-(-)-mandelic acid, preferably crystalline form 1 of Lumateperone tosylate:R-(-)-mandelic acid wherein the process comprises:
a) Providing lumateperone in a mixture of toluene and iso-propylacetate, wherein the ration of toluene and i-propylacetate is from about 9:1 to about 5:4, preferably from about 4:1 to about 5:3, more preferably about 4:3.
b) Adding p-toluene sulfonic acid and R-(-)-mandelic acid;
c) Optionally stirring at a temperature of about 60° C. to about 80° C.;
d) Colling down to a temperature of about 0° C. to about 10° C.;
e) Optionally stirring at a temperature of about 0° C. to about 10° C.; and
f) Optionally isolating crystalline Lumateperone tosylate:R-(-)-mandelic acid.

In any embodiment of this process or in any embodiment or aspect involving these process steps,, steps a, b, c, d, e and/or f may be performed under an inert atmosphere. In any embodiment of this process or in any embodiment or aspect involving these process steps, steps (b)-(f) are carried out under an inert atmosphere (such as under an argon atmosphere.

The present disclosure also provides uses of the said crystalline Lumateperone tosylate:R-(-)-mandelic acid in the preparation of other solid state forms of Lumateperone Tosylate, Lumateperone or other salts of Lumateperone and solid state forms thereof.

The present disclosure also provides the said solid state forms of Lumateperone tosylate:R-(-)-mandelic acid for use in the preparation of other solid state forms of Lumateperone Tosylate, Lumateperone or other salts of Lumateperone and solid state forms thereof.

In another embodiment, the present disclosure encompasses use of crystalline Lumateperone tosylate:R-(-)-mandelic acid or the described solid state form thereof in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

In another embodiment, the present disclosure encompasses crystalline Lumateperone tosylate:R-(-)-mandelic acid or the described solid state form thereof for use in the preparation of pharmaceutical compositions and/or formulations, optionally for the treatment of central nervous system disorders such as schizophrenia, bipolar disorder, depression, sleep and behavioral disturbance in dementia, autism, and other neuropsychiatric disorders.

The present disclosure further provides pharmaceutical compositions including crystalline Lumateperone tosylate:R-(-)-mandelic acid or the described solid state form thereof according to the present disclosure.

In yet another embodiment, the present disclosure encompasses pharmaceutical formulations including crystalline Lumateperone tosylate:R-(-)-mandelic acid or the described solid state form thereof and at least one pharmaceutically acceptable excipient.

The present disclosure encompasses processes to prepare said pharmaceutical formulations of crystalline Lumateperone tosylate:R-(-)-mandelic acid by combining crystalline Lumateperone tosylate:R-(-)-mandelic acid or the described solid state form thereof and at least one pharmaceutically acceptable excipient.

Crystalline Lumateperone tosylate:R-(-)-mandelic acid or the described solid state form thereof can be used as a medicament, in embodiments for the treatment of central nervous system disorders, as specified above.

The present disclosure also provides methods of treating, in embodiments for the treatment of central nervous system disorders, as specified above, by administering a therapeutically effective amount of crystalline Lumateperone tosylate:R-(-)-mandelic acid or of the described solid state form thereof, or at least one of the herein described pharmaceutical compositions or formulations, to a subject suffering from central nervous system disorder or the above specified diseases, or otherwise in need of the treatment.

The present disclosure also provides uses of crystalline Lumateperone tosylate : R-(-)-mandelic acid of the present disclosure, or of the described solid state form thereof, or at least one of the above pharmaceutical compositions or formulations, for the manufacture of medicaments, particularly for the treatment of central nervous system disorders, as specified above.

The present disclosure encompasses processes to prepare said pharmaceutical formulations of Lumateperone Besylate or of crystalline Lumateperone tosylate:R-(-)-mandelic acid by combining the described solid state forms of Lumateperone Besylate or crystalline Lumateperone tosylate:R-(-)-mandelic acid respectively and at least one pharmaceutically acceptable excipient. Excipients are added to the formulation for a variety of purposes. Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, cthyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdonc®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and case the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, crystalline Lumateperone Besylate or crystalline Lumateperone tosylate:R-(-)-mandelic acid and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, preferably a powdered or granulated solid composition of the disclosure, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Lumateperone Besylate or crystalline Lumateperone tosylate:R-(-)-mandelic acid can be administered. Lumateperone Besylate or crystalline Lumateperone tosylate:R-(-)-mandelic acid is preferably formulated for administration to a mammal, preferably a human, by oral administration. Lumateperone Besylate or crystalline Lumateperone tosylate:R-(-)-mandelic acid can be formulated, for example, as a tablet or capsule. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms defined herein as well as the pharmaceutical compositions or formulations of the improved solid state form of Lumateperone Besylate or crystalline Lumateperone tosylate:R-(-)-mandelic acid can be used as medicaments, particularly for the treatment of central nervous system disorders such as schizophrenia, bipolar disorder, depression, sleep and behavioral disturbance in dementia, autism, and other neuropsychiatric disorders.

The present disclosure also provides methods of treating medicaments, particularly for the treatment of central nervous system disorders as specified above including administering a therapeutically effective amount of the improved solid state form of Lumateperone Besylate or crystalline Lumateperone tosylate:R-(-)-mandelic acid of the present disclosure, or at least one of the herein described pharmaceutical compositions or formulations, to a subject suffering from the above specified diseases, or otherwise in need of the treatment.

The present disclosure also provides uses of the improved solid state form of Lumateperone Besylate or crystalline Lumateperone tosylate:R-(-)-mandelic acid of the present disclosure, or at least one of the above pharmaceutical compositions or formulations for the manufacture of medicaments, particularly for the treatment of and/or prophylaxis of central nervous system disorders such as specified above. The present disclosure relates to processes and intermediates for the preparation of Lumateperone and salts thereof

As discussed carlier, the processes discussed in the literature have significant drawbacks. The process disclosed in International Publication No. WO 2000/077022 and in Journal of Medicinal Chemistry 2014, 57, 2670-2682 includes resolution by HPLC to separate Lumateperone from its enantiomer. In the process disclosed in International Publication No. WO 2008/112280 the resolution is performed in an earlier step wherein mandelic acid is used for resolution of intermediate II.

International Publication No. WO 2019/102240 discloses resolution of compound of formula III using (+)-dibenzoyl-D-tartaric acid mono(dimethylamide). However, when the desired diastereomer salt is directly isolated using (+)-dibenzoyl-D-tartaric acid mono(dimethylamide), the product contains 0.04% of the undesired diastercomer as well as 0.14% of further contaminants. When the undesired diastercomer is first isolated as a salt of (−)-dibenzoyl-D-tartaric acid mono(dimethylamide) and only then is the desired diastercomer isolated from the mother liquor as (+)-dibenzoyl-D-tartaric acid mono(dimethylamide) salt, the product contains 0.71% of the undesired diastercomer but still containing 0.31% of three other contaminants. Further, a yield of 45% is reported for the last method whereas for the direct isolation of the desired diastercomer, the yield is only 35%. Advantageously, the process of the present invention, which employs the readily available (−)—O—O′-Di-p-toluoyl-L-tartaric acid as the chiral resolution agent, enables the product of Lumateperone and salts thereof in high purity and without the use of undesirable chromatographic methods.

The process of the present disclosure resolves the cis-racemate compound of formula III using (−)—O—O′-Di-p-toluoyl-L-tartaric acid (designated herein L-DTTA). It has been surprisingly found that while chiral acid such as (S)-mandelic acid, D-DBTA (dibenzoyl-D-tartaric acid), D-malic acid and (1S)-10-camphor sulfonic acid, have failed to resolve compound III, L-DTTA offers an exceptional effect. The process using L-DTTA offers a yield of 44% for the direct isolation of the desired diastereomer using a commercially available acid, with single peak UPLC purity as well as enantiomeric purity of the desired diastercomer.

Further, while the route disclosed in International Publication No. WO 2000/077022 proceeds via a genotoxic nitroso-intermediate, and uses dangerous reagents such as cyanoborohydrides or lithium aluminium hydride as well as toxic solvents like dioxane, the process of the present disclosure offers an advantageous process to produce Lumateperone that uses no genotoxic intermediates with high yield and high purity, using stable intermediates, that can be adapted to production on an industrial scale.

A particular embodiment of the present disclosure uses protecting group that can casily be removed under mild hydrogenation conditions yielding product of high purity and with excellent yield. In contrast, both International Publication No. WO 2000/077022 and International Publication No. WO 2008/112280 disclose the method for removal of ethyl carbamate protecting group under harsh reaction conditions (KOH in n-butanol at reflux) yielding the compound of formula III or its diastereomer as a dark viscous liquid or with low purity (88%). Thus a further advantage of the process of the present disclosure is exhibited.

In one aspect, the present disclosure provides novel intermediates of formulae 2, 3, 4, 5 and 8 that may advantageously be used for the preparation of Lumateperone:

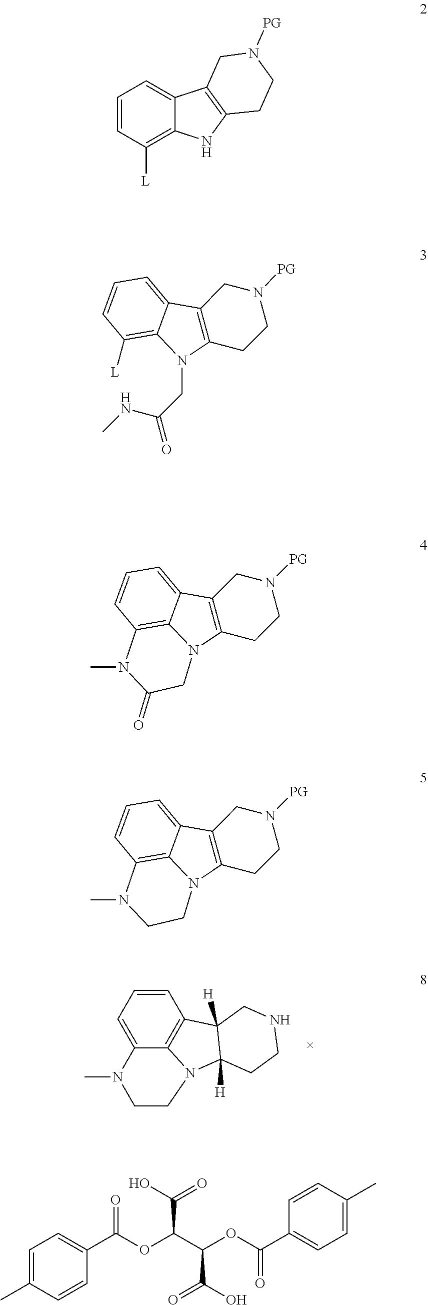

wherein L is a leaving group and PG is a suitable amine protecting group.

Suitable leaving groups may include, but are not limited to, tosylate, triflate or halogen. In embodiments the leaving group is halogen, such as a bromine atom.

Suitable amine protecting groups can be found in Greene and Wuts “Greene's Protective Groups in Organic Synthesis”, 4th Edition, publ. Wiley, 2006.

Suitable amine protecting groups may include, but are not limited to, Fmoc, cbz (carbobenzyloxy), benzyl, trityl, Boc, ethyl carbamate, trifluoroacetyl derivative, phthalic anhydride, succinic anhydride derivative. In embodiments the protecting group is selected from cbz or ethyl carbamate, with the amine protecting group being cbz in some embodiments.

Preferably the compounds of formula 2a, 3a, 4a, 5a or 8 are provided in crystalline form.

In a specific embodiment, the disclosure further provides novel compounds 2a, 3a, 4a and 5a.

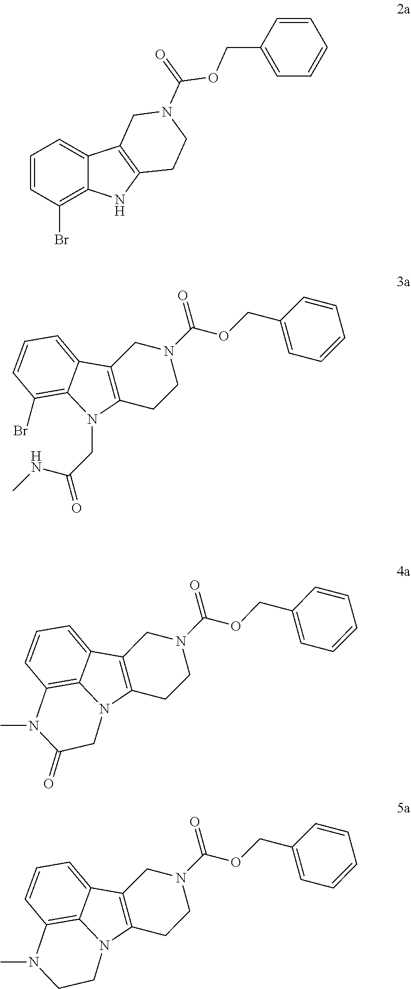 [SMILES: O=C(OCc1ccccc1)N1CCc2[nH]c3c(Br)cccc3c2C1 ; CNC(=O)Cn1c2c(c3cccc(Br)c31)CN(C(=O)OCc1ccccc1)CC2 ; CN1C(=O)Cn2c3c(c4cccc1c42)CN(C(=O)OCc1ccccc1)CC3 ; CN1CCn2c3c(c4cccc1c42)CN(C(=O)OCc1ccccc1)CC3]

In certain embodiments any one of compounds 2a, 3a, 4a, 5a or 8 may be isolated in a solid form, such as a crystalline form.

In another aspect the present disclosure provides the use of any one of the compounds of formulae 2, 3, 4, 5, 8, 2a, 3a, 4a and 5a or the solid state forms thereof in the preparation of Lumateperone.

In another aspect the present disclosure provides processes for preparation of any one of the above compounds and a novel process for preparation of Lumateperone.

In one aspect the disclosure provides a process for preparation of compound 3

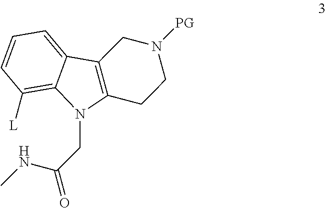

wherein L is a leaving group, such as where L is bromine, and PG is a protecting group as defined above, such as where PG is carbobenzyloxy (cbz) or carboethoxy, in embodiments cbz, including (b) N-alkylating compound 2:

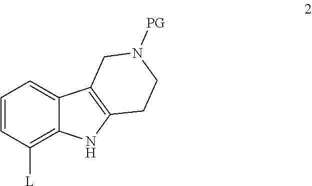

wherein L is a leaving group, such as L is bromine, and PG is a protecting group as defined above, such as PG is carbobenzyloxy (cbz) or carboethoxy, in embodiments cbz to obtain compound 3. In any embodiment of this process or in any embodiment or aspect involving this process step, the N-alkylation is carried out by reacting the compound of formula (2) with a 2-substituted-N-methylacetamide derivative, wherein the 2-position is substituted with a leaving group (such as halo, e.g. chloro or bromo). In any embodiment of this process or in any embodiment or aspect involving this process step, the N-alkylation is carried out by reacting compound (2) with 2-chloro-N-methylacetamide. In any embodiment of this process or in any embodiment or aspect involving this process step, the N-alkylation may be carried out in a polar aprotic solvent, such as dimethylformamide. The N-alkylation may be carried out in presence of potassium phosphate and potassium iodide, The reaction may be carried out at 40° C. to about 70° C., about 40° C. to about 60° C., or at about 50° C.

The starting material of formula 2 as used in any of the processes described herein, may be conveniently prepared by protecting a compound of formula (IV) or a salt thereof. In any embodiment of this process or in any embodiment or aspect involving this process step, the protecting group PG may be carbobenzyloxy (cbz) or carboethoxy:

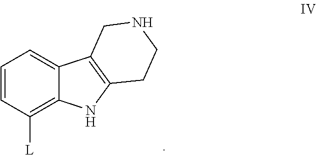

In another aspect the disclosure provides a process for preparation of compound 4

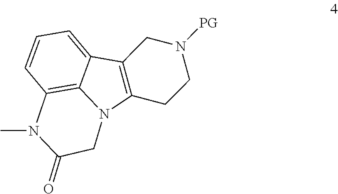

wherein PG is a protecting group as defined above, such as PG is carbobenzyloxy, (cbz) or carboethoxy, in embodiments cbz, wherein the process includes (c) cyclizing compound 3

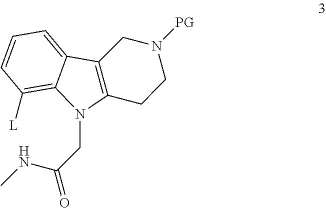

wherein L is a leaving group, such as where L is bromine, and PG is a protecting group as defined above, such as where PG is carbobenzyloxy (cbz) or carboethoxy, in embodiments cbz to obtain compound 4.

The intramolecular cyclization of the compound of formula (3) may conveniently be effected by a palladium-catalysed cyclization or by a copper-catalysed cyclization. In any embodiment of this process or in any embodiment or aspect involving this process step, the compound of formula (3) may be reacted with a palladium complex and a phosphine ligand, in the presence of a base. Alternatively, in any embodiment of this process or in any embodiment or aspect involving this process step, the compound of formula (3) may be reacted with a copper salt and a diamine ligand, in the presence of a base. The reaction may be carried out in an aromatic solvent, such as toluene. The base may be an inorganic base, such as potassium phosphate.

Any suitable palladium complex such as a palladium (0) complex may be used. In any embodiment of this process or in any embodiment or aspect involving this process step, the palladium complex may be palladium tris dibenzylideneacetone

Any suitable phosphine ligand such a mono- or bisphosphine ligand may be used. For example, the phosphine ligand may be 4,5-Bis(diphenylphosphino)-9,9-dimethylxanthene (XantPhos).

In any embodiment of this process or in any embodiment or aspect involving this process step, the cyclisation may be effected by reaction of compound 3 with palladium tri-dibenzylidineacetone and XantPhos, in the presence of potassium phosphate.

In an alternative process, any suitable copper salt such as a copper (I) salt may be used. In any embodiment of this process or in any embodiment or aspect involving this process step, the copper salt may be copper (I) iodide.

Any suitable diamine ligand such as an alkyldiamine may be used. For example, the alkyldiamine may be (N,N′-Dimethylethylenediamine) (DMEDA).

In any embodiment of this process or in any embodiment or aspect involving this process step, the cyclisation may be effected by reaction of compound 3 with copper (I) iodide and DMEDA in the presence of potassium phosphate.

In any embodiment of this process or in any embodiment or aspect involving this process step, the reaction may be carried out at a temperature of about 80° C. to about 120° C., preferably at about 100° C. The starting compound of formula 3 can be suitably prepared by the process as described in any embodiment discussed herein.

In another aspect the disclosure relates to a process for preparation of compound 5

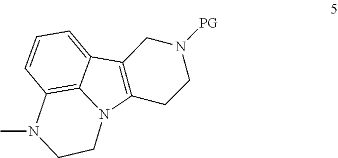

wherein PG is a protecting group as defined above, such as PG is carbobenzyloxy (cbz) or carboethoxy, in embodiments cbz, wherein the process includes (d) reducing compound 4

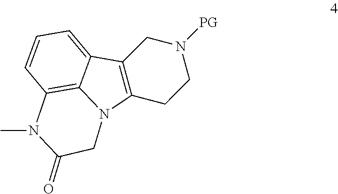

wherein PG is a protecting group as defined above, such as PG is carbobenzyloxy, (cbz) or carboethoxy, in embodiments cbz, to obtain compound 5.

In any embodiment of this process or in any embodiment or aspect involving this process step, the reduction is carried out in the presence of a reducing agent selected from a hydrogen, a hydride or a borohydride. In any embodiment of this process or in any embodiment or aspect involving this process step, the reducing agent may be a hydride, such as BH₃-THF. In any embodiment of this process or in any embodiment or aspect involving this process step, the reaction is carried out in the presence of a polar aprotic solvent, such as a polar aprotic solvent selected from the group consisting of ethers, esters and nitriles. In any embodiment of this process or in any embodiment or aspect involving this process step, the reaction may be carried out using a solvent comprising THF. The starting compound of formula 4 can be conveniently prepared by a process as described in any embodiment herein. The starting compound of formula 4 can be conveniently prepared by a process as described in any embodiment herein.

In another aspect the disclosure relates to a process for preparation of compound 6

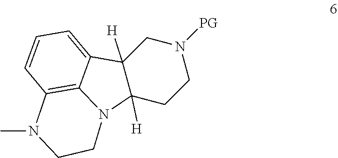

wherein PG is a protecting group as defined above, such as PG is carbobenzyloxy (cbz) or carboethoxy, in embodiments cbz including (e) reducing compound 5

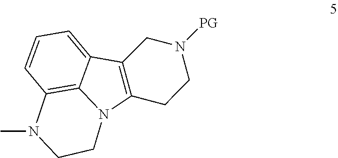

wherein PG is a protecting group as defined above, such as PG is carbobenzyloxy (cbz) or carboethoxy, in embodiments cbz, to obtain compound 6.

In any embodiment of this process or in any embodiment or aspect involving this process step, the compound of formula (5) may be reacted with a reducing agent, such as hydrogen, a hydride or a borane. In any embodiment of this process or in any embodiment or aspect involving this process step, the reducing agent may be a hydride, such as sodium borohydride. In any embodiment of this process or in any embodiment or aspect involving this process step, the reaction may be carried out in the presence of an acid, such as hydrochloric acid, acetic acid or trifluoroacetic acid (TFA). In any embodiment of this process or in any embodiment or aspect involving this process step, the acid may be TFA. The reaction is suitably carried out in the presence of a polar aprotic solvent such as a polar aprotic solvent selected from the group consisting of an ether (such as THF); an ester and a nitrile (such as acetonitrile). In any embodiment of this process or in any embodiment or aspect involving this process step, the reaction may be carried out in acetonitrile. The starting compound of formula 5 can be conveniently prepared by a process as described in any embodiment herein.

The invention further relates to a process for the preparation of a compound of formula 8

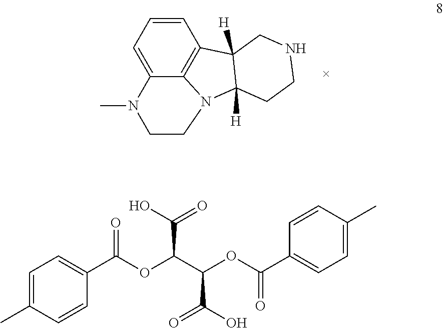

### Including:

deprotecting a compound of formula 6:

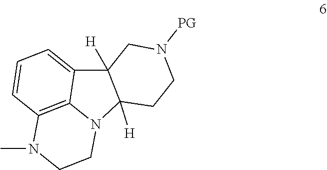

wherein PG is a protecting group as defined above, such as PG is carbobenzyloxy (cbz) or carboethoxy, in embodiments cbz,
to obtain the compound rac-7:

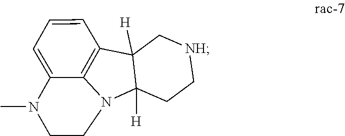 [SMILES: CN1CCN2c3c(cccc31)C1CNCCC12]

and
reacting the compound rac-7 with (-)-di-p-toluoyl-L-tartaric acid (L-DTTA).

In any embodiment of this process or in any embodiment or aspect involving this process step, the reaction with L-DTTA may be carried out in methanol or a combination of methanol with water or dichloromethane. Conveniently, methanol may be used.

In any embodiment of this process or in any embodiment or aspect involving this process step, the reaction with L-DTTA may be carried out in methanol and compound 8 is precipitated from the reaction mixture.

The starting compound 6 can be prepared by a process as described in any of the embodiments discussed herein.

The process advantageously provides a convenient synthesis of compound 8 from compound 6, wherein the intermediate rac-7 need not be isolated as a solid (e.g. the compound rac-7 can be obtained from the deprotection of compound 6, without isolation as a solid, and/or without purification after the reaction work up. Thus, in any embodiment of this process or in any embodiment or aspect involving these process steps, the conversion of compound (6) to compound (8) can be carried out without purification of the compound rac-7.

Unless otherwise indicated, the reference to the term “without purification” in relation to a compound according to any aspect or embodiment of the invention, means that the compound obtained from a reaction is not subjected to purification steps, such as chromatography, crystallisation/recrystallization. Thus the term “without purification” refers to a compound that is obtained from the reaction mixture after any work-up step, but without purification. Typically such a compound may be referred to as a “crude compound”. Such a compound may be in the form of a solid (e.g. obtained after work-up and removal of any solvent from the work up) or the compound may be in a solution obtained after work-up without removal of solvent, or as a wet solid obtained after work-up and partial removal of solvent.

Thus, in any embodiment of this process or in any embodiment or aspect involving these process steps, the compound 6 can be prepared as described in any embodiment above. The reaction steps of the conversion of compound 5 to compound 8 may be carried out without purification of compound 6 and/or compound rac-7.

In another aspect, the disclosure provides a process for preparation of Lumateperone (1) or salt thereof including:

(i) basifying compound 8

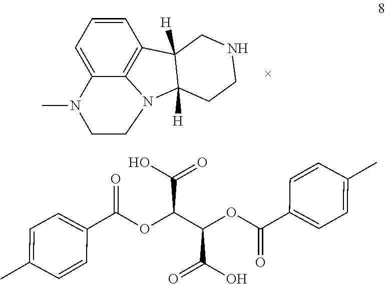

to obtain the 6bR, 10a S diastereomer 7

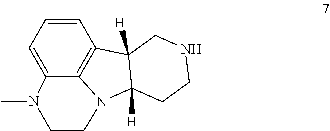 [SMILES: CN1CCN2c3c(cccc31)[C@@H]1CNCC[C@@H]12]

(ii) alkylating 7 to obtain lumateperone;
(iii) optionally converting Lumateperone to a salt thereof; and
(iv) optionally converting the salt to a different salt of Lumateperone.

In embodiments, step (iii) includes converting lumateperone to lumateperone besylate, preferably Lumateperone dibesylate.

In embodiments, step (iv) includes converting Lumateperone besylate, in embodiments lumateperone dibesylate to Lumateperone tosylate or solid states form thereof such as Lumateperone tosylate:R-(-)-mandelic acid. Thus, in any embodiment of this process or in any embodiment or aspect involving these process steps, the starting compound 8 may be prepared by a process as described in any of the embodiments discussed herein.

The present invention provides a convenient conversion of compound (8) to Lumateperone whereby the intermediate compound (7) can be carried out without purification of compound 7.

In any aspect or embodiment of the present invention, the leaving group L in any of the intermediates is preferably Br. The protecting group PG is preferably cbz, Suitably, the leaving group L is Br and the protecting group is cbz.

In another aspect, there is provided a process for preparation of Lumateperone or salt thereof wherein the process comprises:
(i) reducing a compound of formula 5:

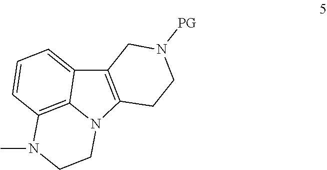

wherein PG is a protecting group, preferably wherein PG is carbobenzyloxy (cbz) or carboethoxy, most preferably cbz, to obtain a compound of formula 6:

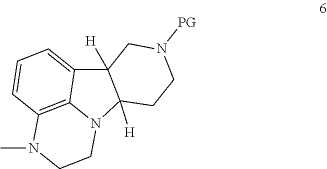

(ii) removing the protecting group to obtain the compound rac-7:

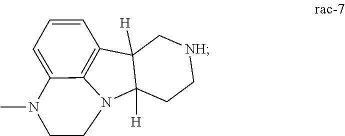 [SMILES: CN1CCN2c3c(cccc31)C1CNCCC12]

(iii) reacting the compound rac-7 with L-DTTA to obtain a compound of formula 8:

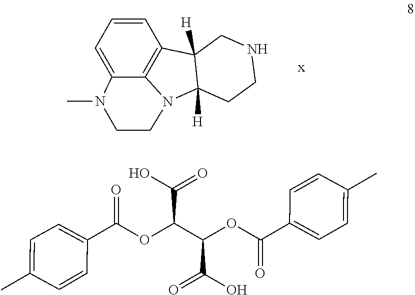

(iv) basifying compound 8 to obtain a compound of formula 7:

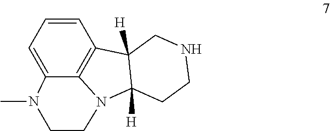 [SMILES: CN1CCN2c3c(cccc31)[C@@H]1CNCC[C@@H]12]

(v) alkylating the compound (7) with 4-chloro-4′-fluorobutyrophenone to obtain Lumateperone; and
(vi) optionally converting Lumateperone to a salt thereof or a solid state form.

In another aspect the disclosure provides a process for preparation of Lumateperone (1) or salt thereof including:

(i) basifying compound 8

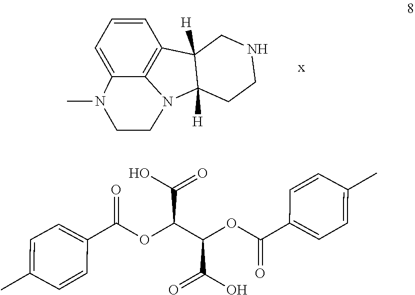

to obtain the 6bR, 10a S diastereomer 7

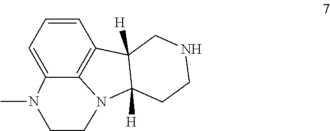 [SMILES: CN1CCN2c3c(cccc31)[C@@H]1CNCC[C@@H]12]

(ii) alkylating 7 with 4-chloro-4′-fluorobutyrophenone to obtain lumateperone;
(iii) converting Lumateperone to Lumateperone dibesylate; and
(iv) converting Lumateperone dibesylate to lumateperone or another salt thereof.

In embodiments, step (iv) includes converting Lumateperone besylate, in embodiments lumateperone dibesylate to Lumateperone tosylate or solid states form thereof such as Lumateperone tosylate:R-(-)-mandelic acid.

In another aspect the disclosure provides a process for preparation of Lumateperone tosylate:R-(-)-mandelic acid including:

(i) basifying compound 8

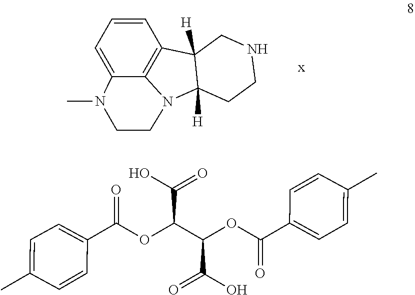

to obtain the 6bR, 10a S diastereomer 7

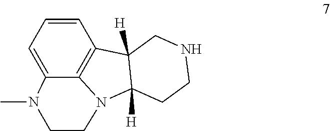 [SMILES: CN1CCN2c3c(cccc31)[C@@H]1CNCC[C@@H]12]

(ii) alkylating 7 with 4-chloro-4′-fluorobutyrophenone to obtain lumateperone;
(iii) converting Lumateperone to Lumateperone dibesylate; and
(iv) converting Lumateperone dibesylate to lumateperone tosylate:R-(-)-mandelic acid.

In one aspect the disclosure provides a process for preparation of compound 3

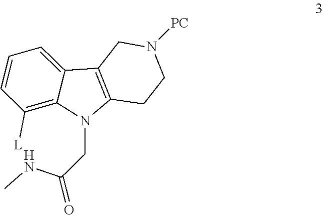

wherein L is a leaving group, such as where L is bromine, and PG is a protecting group as defined above, such as where PG is carbobenzyloxy (cbz) or carboethoxy, in embodiments cbz, including:
a) protecting compound IV or salt thereof

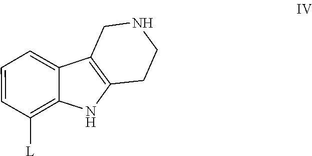

wherein L is a leaving group, such as L is bromine, and PG is a protecting group as defined above, such as PG is carbobenzyloxy (cbz) or carboethoxy, in embodiments cbz, to obtain compound 2

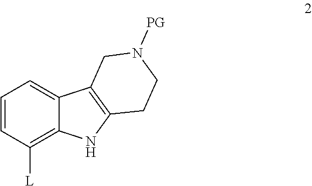

wherein L is a leaving group, such as L is bromine, and PG is a protecting group as defined above, such as PG is carbobenzyloxy (cbz) or carboethoxy, in embodiments cbz; and
b) N-alkylating compound 2 to obtain compound 3.

In another aspect the disclosure provides a process for preparation of compound 4

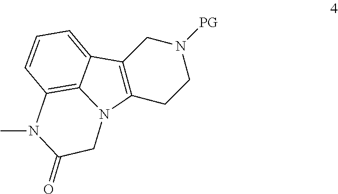

wherein PG is a protecting group as defined above, such as PG is carbobenzyloxy, (cbz) or carboethoxy, in embodiments cbz, wherein the process includes:
a) protecting compound IV or salt thereof

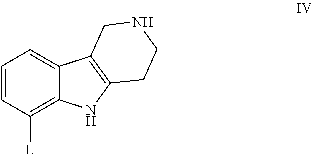

wherein L is a leaving group, such as L is bromine, and PG is a protecting group as defined above, such as PG is carbobenzyloxy (cbz) or carboethoxy, in embodiments cbz, to obtain compound 2

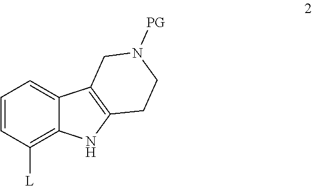

wherein L is a leaving group, such as L is bromine, and PG is a protecting group as defined above, such as PG is carbobenzyloxy (cbz) or carboethoxy, in embodiments cbz;
b) N-alkylating compound 2 to obtain compound 3

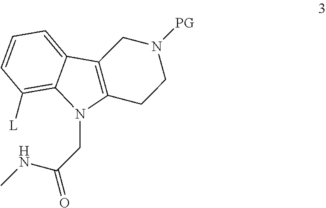

wherein L is a leaving group, such as L is bromine, and PG is a protecting group as defined above, such as PG is carbobenzyloxy (cbz) or carboethoxy, in embodiments cbz; and
c) cyclizing compound 3 to obtain compound 4.

In another aspect the disclosure relates to a process for preparation of compound 5

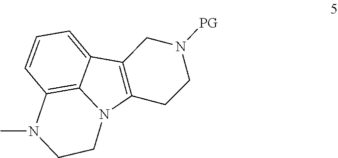

wherein PG is a protecting group as defined above, such as PG is carbobenzyloxy (cbz) or carboethoxy, in embodiments cbz, wherein the process includes:
a) protecting compound IV or salt thereof

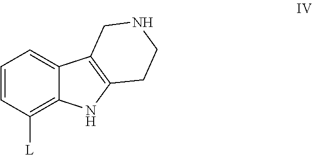

wherein L is a leaving group, such as L is bromine, and PG is a protecting group as defined above, such as PG is carbobenzyloxy (cbz) or carboethoxy, in embodiments cbz, to obtain compound 2

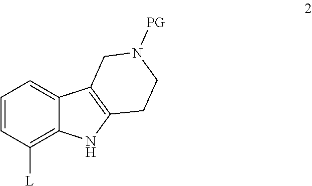

wherein L is a leaving group, such as L is bromine, and PG is a protecting group as defined above, such as PG is carbobenzyloxy (cbz) or carboethoxy, in embodiments cbz;
b) N-alkylating compound 2 to obtain compound 3

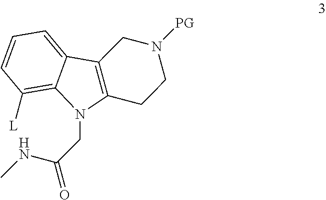

wherein L is a leaving group, such as L is bromine, and PG is a protecting group as defined above, such as PG is carbobenzyloxy (cbz) or carboethoxy, in embodiments cbz;
c) cyclizing compound 3 to obtain compound 4

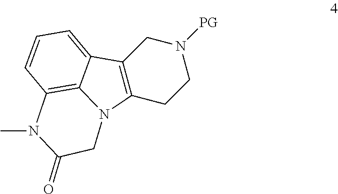

wherein PG is a protecting group as defined above, such as PG is carbobenzyloxy (cbz) or carboethoxy, in embodiments cbz; and
d) reducing compound 4 to obtain compound 5.

In another aspect the disclosure provides a process for preparation of Lumateperone or salt thereof, wherein the process includes:
e) reducing compound 5

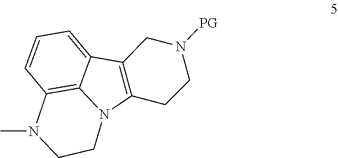

wherein PG is a protecting group as defined above, such as PG is carbobenzyloxy (cbz) or carboethoxy, in embodiments cbz to obtain compound 6

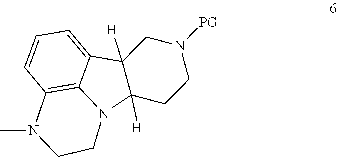

wherein PG is a protecting group as defined above, such as PG is carbobenzyloxy (cbz) or carboethoxy, in embodiments cbz;
f) removing the protecting group to obtain racemic rac-7

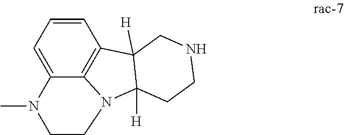 [SMILES: CN1CCN2c3c(cccc31)C1CNCCC12]

g) reacting racemic compound 7 with L-DTTA to obtain the L-DTTA salt of the 6bR, 10aS diastereomer (7), designated compound 8:

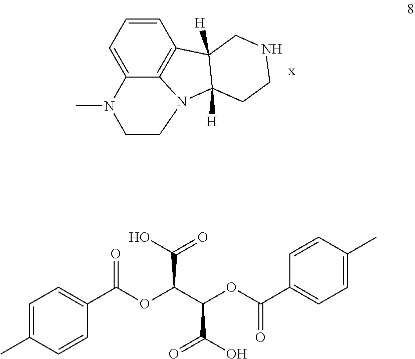

h) basifying compound 8 to obtain the 6bR, 10aS (7):

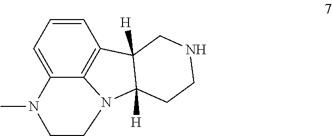 [SMILES: CN1CCN2c3c(cccc31)[C@@H]1CNCC[C@@H]12]

i) Alkylating the 6bR, 10aS diastereomer (7) with 4-chloro-4′-fluorobutyrophenone to obtain Lumateperone; and
j) optionally converting Lumateperone to a salt thereof or a solid state form thereof.

In some embodiments, any one or more of compounds 7, rac-7 and/or 6 may be obtained from the reaction mixture as a solution (e.g. following work up), which may be directly used in the next step without further purification, or the intermediate compound can be isolated as solid (e.g. by removal or partial removal of solvent(s) following work-up, and used in the subsequent step without further purification.

In some embodiments, the disclosure relates to the above processes wherein any one of compounds 7, rac-7 and/or 6 is kept in solution and directly used in the next step or isolated as solids and later solubilized to be used in the next step without further purification. Thus, in any embodiment, any one or all of the compounds 7, rac-7, and 6, are used in a subsequent reaction step without purification, i.e. the compound(s) are obtained as a solution from any reaction work up, or optionally obtained as a solid or wet solid after work up and solvent removal, and used directly in a subsequent reaction step.

In certain embodiments, the disclosure relates to the above process wherein compound 7 is kept in solution and directly used in the next step or isolated as a solid and later solubilized to be used in the next step without further purification, i.e. the compound 7 is obtained as a solution following reaction work up, or optionally obtained as a solid or wet solid following work up and solvent removal, and used directly in a subsequent reaction step.

In certain embodiments, the disclosure relates to the above process wherein compound 7 is kept in solution and directly used in the next step or isolated as a solid and later solubilized to be used in the next step without further purification, and wherein any one of compounds rac-7 and/or 6 is/are formed and used in the next step without further purification and/or compound rac-7 is afforded in a methanolic solution and is directly affected in the next step to the resolution conditions.

The N-alkylation step to prepare the compound of formula (3) is preferably carried out by reaction with a 2-substituted-N-methylacetamide derivative, wherein the 2-position is substituted with a leaving group. The leaving group may be any suitable group, such as halo. Thus, in any embodiment of this process or in any embodiment or aspect involving this process step, the leaving group is chloro or bromo, preferably chloro. In any embodiment of this process or in any embodiment or aspect involving this process step, the compound of formula (3) is prepared by reaction of (2) with 2-chloro-N-methylacetamide.

In embodiments the N-alkylation reaction in step b) is performed in a polar aprotic solvent, such as DMF and in the presence of potassium phosphate and potassium iodide. In specific embodiments the reaction is performed at about 40° C. to about 60° C., in embodiments at about 50° C.

In embodiments the cyclization reaction in step c) is performed in an aromatic solvent, in the presence of a base, a palladium precursor and a ligand. Examples of the aromatic solvent may include, but are not limited to xylene or toluene, in embodiments the aromatic solvent is toluene.

Examples of the base may include, but are not limited to inorganic bases such as potassium carbonate, sodium carbonate, potassium acetate, potassium phosphate, in embodiments the base is potassium phosphate. Examples of the ligand may include, but are not limited to any mono- or bisphosphine ligand, in embodiments the ligand is XantPhos. In some embodiments the reaction is performed at about 80° C. to about 120° C., in embodiments at about 100° C.

In embodiments the reduction in step d) is performed in a polar aprotic solvent and in the presence of a reducing agent. Examples of the polar aprotic solvent may include, but are not limited to ethers, esters, nitriles, in embodiments the solvent is THF. Examples of the reducing agent may include, but are not limited to hydrides or boranes, in embodiments the reducing agent is BH₃-THF.

In embodiments the reduction in step e) is performed in a polar aprotic solvent and in the presence of a reducing agent and an acid, such as TFA. Examples of the polar aprotic solvent may include, but are not limited to ethers, esters, nitriles, in embodiments ACN. Examples of the reducing agent may include, but are not limited to hydrogen, hydrides or boranes in combination with an acid, in embodiments the reducing agent is NaBH₄. Examples of a suitable acid may include, but are not limited to hydrochloric acid, acetic acid or TFA, in embodiments the acid is TFA.

In embodiments the resolution step is done in methanol or combinations of methanol with water or dichloromethane. In some embodiments resolution is done in methanol and compound 8 is precipitated.

The process of the disclosure starting from (V) to Lumateperone may be summarized as depicted in scheme 4:

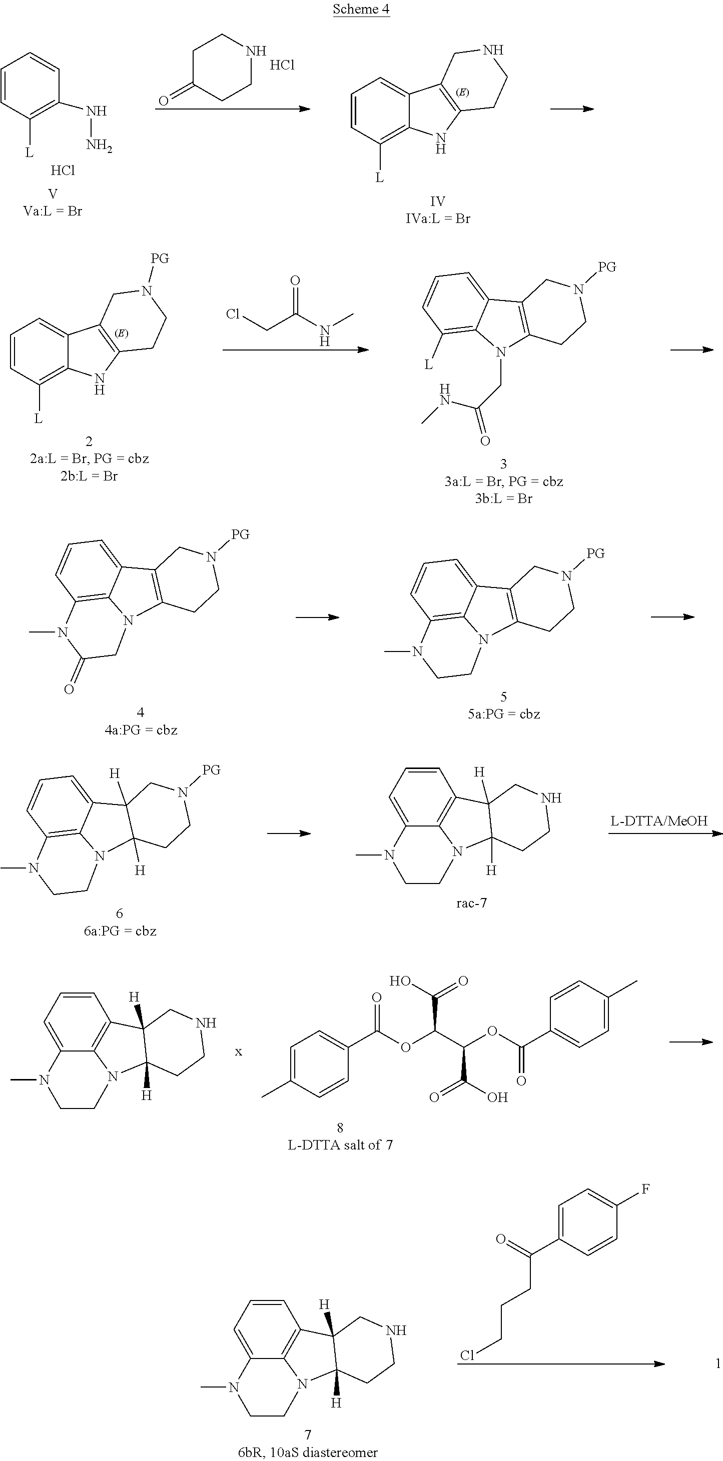

In one embodiment, the disclosure relates to the above process for preparation of Lumateperone as described above wherein the leaving group is bromine.

In another embodiment the disclosure relates to the above process for preparation of Lumateperone as described above wherein the leaving group is bromine and the protecting group is cbz.

In certain embodiments the disclosure relates to the above process for preparation of Lumateperone as described above wherein the leaving group is bromine and the protecting group is cbz and wherein any one of compounds 6a and/or rac-7 and/or 7 may be formed and used in the next step without further purification. In certain embodiments compounds 6a and/or rac-7 and/or 7 may be kept in solution and directly used in the next step or isolated as solids and later solubilized to be used in the next step without further purification.

In a certain embodiment the disclosure relates to the above process for preparation of Lumateperone as described above wherein the leaving group is bromine and the protecting group is cbz and wherein compound 7 is formed and used in the next step without further purification.

In a certain embodiment the disclosure relates to the above process for preparation of Lumateperone as described above wherein the leaving group is bromine and the protecting group is cbz and wherein compound 7 is formed and used in the next step without further purification and wherein compound 6a is formed and used in the next step without further purification and/or compound rac-7 is afforded in a methanolic solution and is directly affected in the next step to the resolution conditions.

In a particular embodiment the disclosure further provides a process for preparation of Lumateperone or salt thereof including:
e) reducing compound 5a

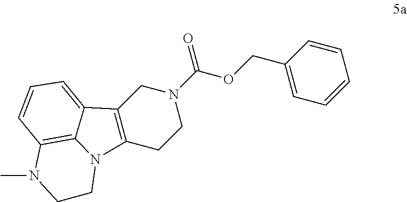 [SMILES: CN1CCn2c3c(c4cccc1c42)CN(C(=O)OCc1ccccc1)CC3]

To obtain compound 6a

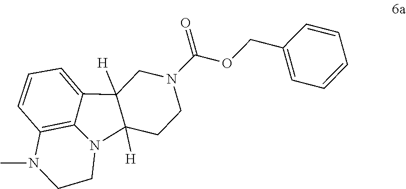 [SMILES: CN1CCN2c3c(cccc31)C1CN(C(=O)OCc3ccccc3)CCC12]

f) removing the protecting group to obtain racemic rac-7

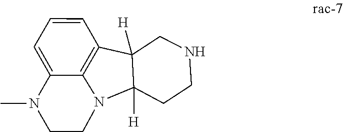 [SMILES: CN1CCN2c3c(cccc31)C1CNCCC12]

g) reacting racemic compound 7 with L-DTTA to obtain the L-DTTA salt of the 6bR, 10aS diastereomer (7), designated compound 8:

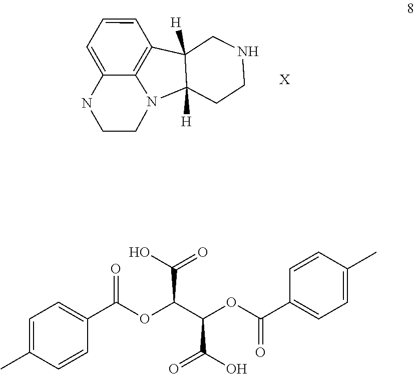

h) basifying compound 8 to obtain the 6bR, 10aS (7):

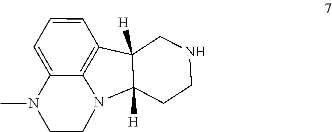 [SMILES: CN1CCN2c3c(cccc31)[C@@H]1CNCC[C@@H]12]

i) alkylating the 6bR, 10aS diastereomer (7) with 4-chloro-4′-fluorobutyrophenone to obtain Lumateperone; and
j) optionally converting Lumateperone to a salt thereof or a solid state form thereof.

In certain embodiments the disclosure relates to the above process wherein any one of compounds 6a and/or rac-7 and/or 7 may be formed and used in the next step without further purification. In certain embodiments compounds 6a and/or rac-7 and/or 7 may be kept in solution and directly used in the next step or isolated as solids and later solubilized to be used in the next step without further purification. Thus, in any embodiment of this process, any one or all of the compounds 7, rac-7, and 6, are used in a subsequent reaction step without purification, i.e. the compound(s) are obtained as a solution from any reaction work up, or optionally obtained as a solid or wet solid after work up and solvent removal, and used directly in a subsequent reaction step.

In a certain embodiment the disclosure relates to the above process for preparation of Lumateperone wherein compound 7 is formed and used in the next step without further purification, i.e. the compound 7 is obtained as a solution following reaction work up, or optionally obtained as a solid or wet solid following work up and solvent removal, and used directly in a subsequent reaction step.

In a certain embodiment the disclosure relates to the above process for preparation of Lumateperone wherein compound 7 is formed and used in the next step without further purification and wherein compound 6a is formed and used in the next step without further purification and/or compound rac-7 is afforded in a methanolic solution and is directly affected in the next step to the resolution conditions.

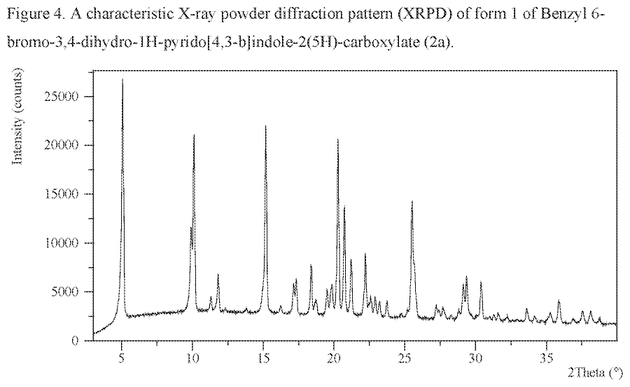

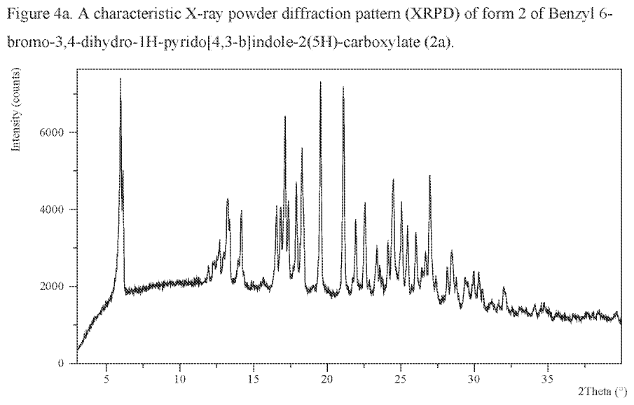

The present disclosure relates to a crystalline form of Benzyl 6-bromo-3,4-dihydro-1H-pyrido[4,3-b]indole-2(5H)-carboxylate (compound 2a), designated form 1. The crystalline form 1 of compound 2a may be characterized by data selected from one or more of the following: an X-ray powder diffraction pattern substantially as depicted in FIG. 4; an X-ray powder diffraction pattern having peaks at 5.1, 10.1, 15.2, 20.3 and 25.5±0.2 degrees 2-theta±0.2 degrees 2-theta; and combinations of these data.

Crystalline form 1 of compound 2a may be further characterized by an X-ray powder diffraction pattern having peaks at 5.1, 10.1, 15.2, 20.3 and 25.5±0.2 degrees 2-theta±0.2 degrees 2-theta, and also having any one, two, three, four or five additional peaks selected from 11.8, 20.7, 21.2, 22.2 and 30.4 degrees 2-theta±0.2 degrees 2-theta.

The present disclosure relates to a crystalline form of benzyl 6-bromo-3,4-dihydro-1H-pyrido[4,3-b]indole-2(5H)-carboxylate (compound 2a), designated form 2. The crystalline form 2 of compound 2a may be characterized by data selected from one or more of the following: an X-ray powder diffraction pattern substantially as depicted in FIG. 4a; an X-ray powder diffraction pattern having peaks at 6.0, 17.1, 18.3, 19.6 and 24.5±0.2 degrees 2-theta±0.2 degrees 2-theta; and combinations of these data.

Crystalline form 2 of compound 2a may be further characterized by an X-ray powder diffraction pattern having peaks at 6.0, 17.1, 18.3, 19.6 and 24.5±0.2 degrees 2-theta±0.2 degrees 2-theta, and also having any one, two, three, four or five additional peaks selected from 14.2, 16.6, 18.0, 26.0 and 27.0 degrees 2-theta±0.2 degrees 2-theta.

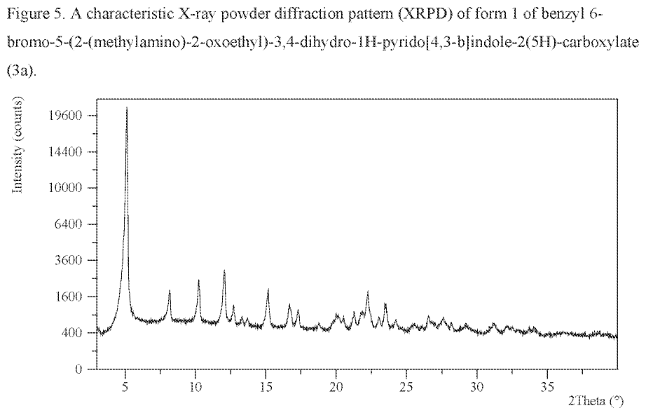

The present disclosure relates to a crystalline form of benzyl 6-bromo-5-(2-(methylamino)-2-oxocthyl)-3,4-dihydro-1H-pyrido[4,3-b]indole-2(5H)-carboxylate (compound 3a), designated form 1. The crystalline form 1 of compound 3a may be characterized by data selected from one or more of the following: an X-ray powder diffraction pattern substantially as depicted in FIG. 5; an X-ray powder diffraction pattern having peaks at 5.1, 10.2, 12.1, 15.2 and 22.2±0.2 degrees 2-theta±0.2 degrees 2-theta; and combinations of these data.

Crystalline form 1 of compound 3a may be further characterized by an X-ray powder diffraction pattern having peaks at 5.1, 10.2, 12.1, 15.2 and 22.2±0.2 degrees 2-theta±0.2 degrees 2-theta, and also having any one, two, three, four or five additional peaks selected from 8.2, 12.7, 16.7, 17.3 and 23.5 degrees 2-theta±0.2 degrees 2-theta.

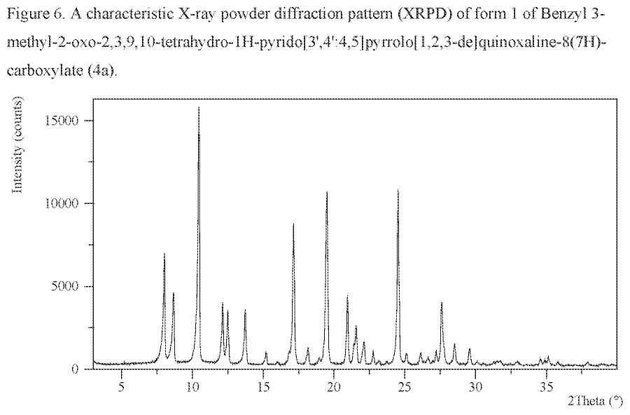

The present disclosure relates to a crystalline form of Benzyl 3-methyl-2-oxo-2,3,9,10-tetrahydro-1H-pyrido[3′,4′:4,5]pyrrolo[1,2,3-de]quinoxaline-8(7H)-carboxylate (compound 4a), designated form 1. The crystalline form 1 of compound 4a may be characterized by data selected from one or more of the following: an X-ray powder diffraction pattern substantially as depicted in FIG. 6; an X-ray powder diffraction pattern having peaks at 8.0, 10.5, 17.1, 19.5 and 24.5±0.2 degrees 2-theta±0.2 degrees 2-theta; and combinations of these data.

Crystalline form 1 of compound 4a may be further characterized by an X-ray powder diffraction pattern having peaks at 8.0, 10.5, 17.1, 19.5 and 24.5±0.2 degrees 2-theta±0.2 degrees 2-theta, and also having any one, two, three, four or five additional peaks selected from 8.7, 13.7, 21.0, 21.6 and 27.6 degrees 2-theta±0.2 degrees 2-theta.

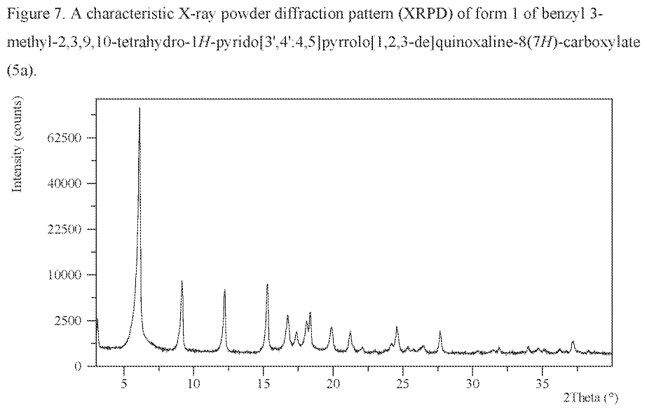

The present disclosure relates to a crystalline form of benzyl 3-methyl-2,3,9,10-tetrahydro-1H-pyrido[3′,4′:4,5]pyrrolo[1,2,3-de]quinoxaline-8(7H)-carboxylate (compound 5a), designated form 1. The crystalline form 1 of compound 5a may be characterized by data selected from one or more of the following: an X-ray powder diffraction pattern substantially as depicted in FIG. 7; an X-ray powder diffraction pattern having peaks at 6.1, 9.2, 12.2, 15.3 and 24.6±0.2 degrees 2-theta±0.2 degrees 2-theta; and combinations of these data.

Crystalline form 1 of compound 5a may be further characterized by an X-ray powder diffraction pattern having peaks at 6.1, 9.2, 12.2, 15.3 and 24.6±0.2 degrees 2-theta±0.2 degrees 2-theta, and also having any one, two, three, four or five additional peaks selected from 16.8, 18.4, 19.9, 21.2 and 27.7 degrees 2-theta±0.2 degrees 2-theta.

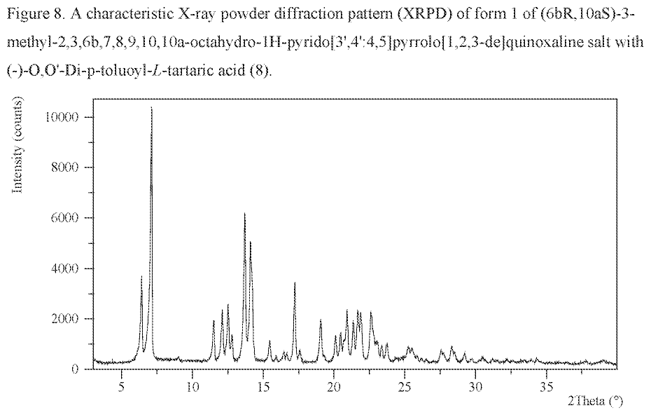

The present disclosure relates to a crystalline form of (6bR, 10aS)-3-methyl-2,3,6b,7,8,9,10,10a-octahydro-1H-pyrido[3′,4′:4,5]pyrrolo[1,2,3-de]quinoxaline salt with (-)—O,O′-Di-p-toluoyl-L-tartaric acid (compound 8), designated form 1. The crystalline form 1 of compound 8 may be characterized by data selected from one or more of the following: an X-ray powder diffraction pattern substantially as depicted in FIG. 8; an X-ray powder diffraction pattern having peaks at 7.1, 12.5, 13.7, 17.2 and 20.9±0.2 degrees 2-theta±0.2 degrees 2-theta; and combinations of these data.

Crystalline form 1 of compound 8 may be further characterized by an X-ray powder diffraction pattern having peaks at 7.1, 12.5, 13.7, 17.2 and 20.9±0.2 degrees 2-theta±0.2 degrees 2-theta, and also having any one, two, three, four or five additional peaks selected from 6.4, 11.5, 14.1, 19.1 and 22.6 degrees 2-theta±0.2 degrees 2-theta.

In another aspect the present disclosure provides Lumateperone or salts thereof produced by the processes of the present disclosure.

The present disclosure further provides: Lumateperone or salt thereof, such as Lumateperone dibenzenesulfonate or lumateperone tosylate or R-(-)-mandelic acid, obtainable by a process according to any embodiment described herein. The Lumateperone or salt thereof, such as Lumateperone dibenzenesulfonate or lumateperone tosylate or R-(-)-mandelic acid may have a total impurity content of not more than 1% area percent, not more than 0.8% area percent, or not more than 0.6% area percent as measured by UPLC.

The present disclosure further provides Lumateperone tosylate:R-(-)-mandelic acid, obtainable by a process according to any embodiment described herein. The Lumateperone tosylate:R-(-)-mandelic acid may advantageously have a total impurity content of not more than 0.3% area percent, or not more than 0.2% area percent, more preferably not more than 0.1% arca percent, or not more than 0.08% area percent, as measured by UPLC.

The present disclosure further provides Lumateperone, obtainable by a process according to any embodiment described herein. Advantageously, the Lumateperone may have s an enantiomeric excess of more than 99% area percent, preferably more than 99.5% area percent, more preferably more than 99.9% area percent, as measured by UPLC.

The present disclosure provides Lumateperone tosylate:R-(-)-mandelic acid, obtainable by a process according to any embodiment described herein, optionally wherein the Lumateperone tosylate:R-(-)-mandelic acid, has an enantiomeric excess of more than 99% area percent, or more than 99.5% area percent, or more than 99.9% area percent, as measured by UPLC.

The present disclosure also encompasses the use of the Lumateperone or salts thereof prepared by the processes of the present disclosure for the preparation of pharmaceutical compositions of Lumateperone or salts thereof.

The present disclosure includes processes for preparing the above mentioned pharmaceutical compositions. The processes include combining the Lumateperone or salt thereof prepared by the processes of the present disclosure or salts thereof with at least one pharmaceutically acceptable excipient.

Lumateperone or salts thereof prepared by the processes of the present disclosure and the pharmaceutical compositions of Lumateperone or salts thereof prepared by the processes of the present disclosure can be used as medicaments, in embodiments for the treatment of central nervous system (CNS) disorders.

The present disclosure also provides methods for the treatment of central nervous system (CNS) disorders, by administering a therapeutically effective amount of Lumateperone or salts thereof prepared by the processes of the present disclosure, or at least one of the above pharmaceutical compositions, to a subject in need of the treatment.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### XRPD Method (FIG. 1)

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan. The described peak positions were determined with or without using silicon powder as an internal standard in an admixture with the sample measured. The position of the silicon (Si) peak was corrected to silicone theoretical peak: 28.45 degrees two theta and the positions of the measured peaks were corrected respectively.

### XRPD Method (FIGS. 3-9, 14, 18 and 22)

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan. The described peak positions were determined without using silicon powder as an internal standard in an admixture with the sample measured.

### DSC Method

DSC analysis was performed on instrument Q1000 MDSC TA with a heating rate of 10° C./min and under nitrogen flow of 50 mL/min. Standard aluminum closed pan (with hole) was used, sample mass was 1-5 mg.

### TGA Method

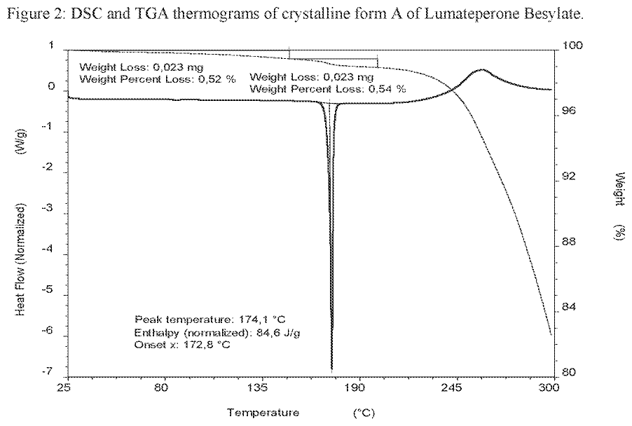

### TGA Method (FIG. 2)

TGA analysis was performed on instruments Mettler Toledo TG-DSC 1 with a heating rate of 10° C./min and under nitrogen flow of 30 mL/min. Standard aluminum open pan was used, sample mass was 1-10 mg

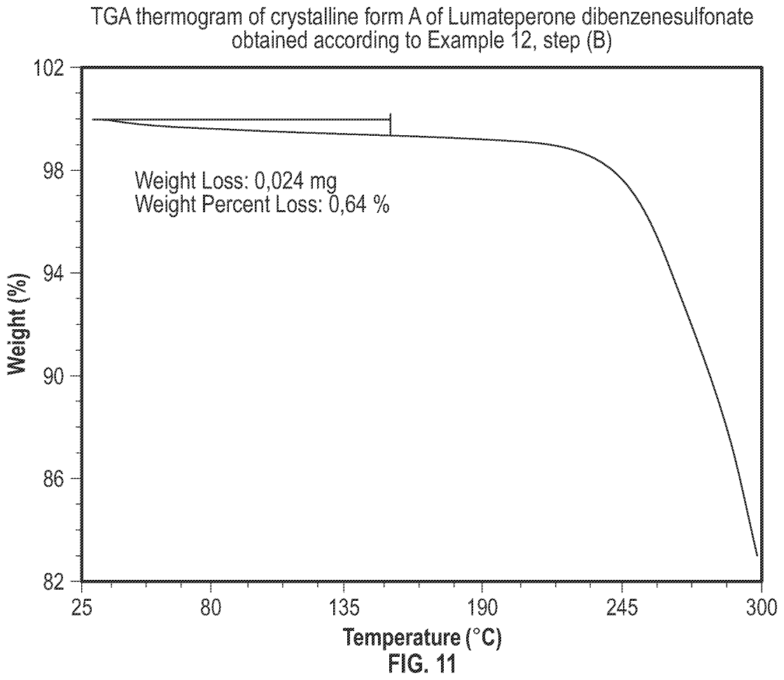

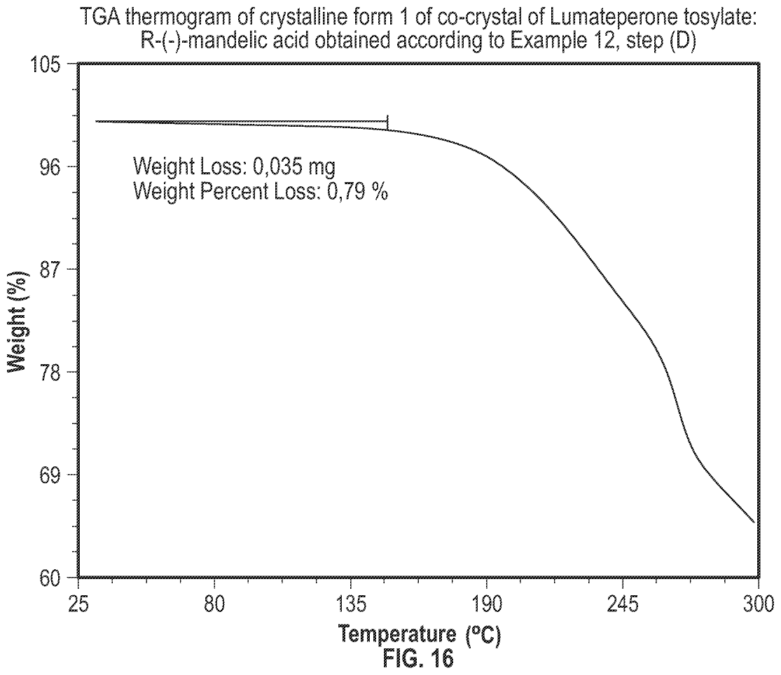

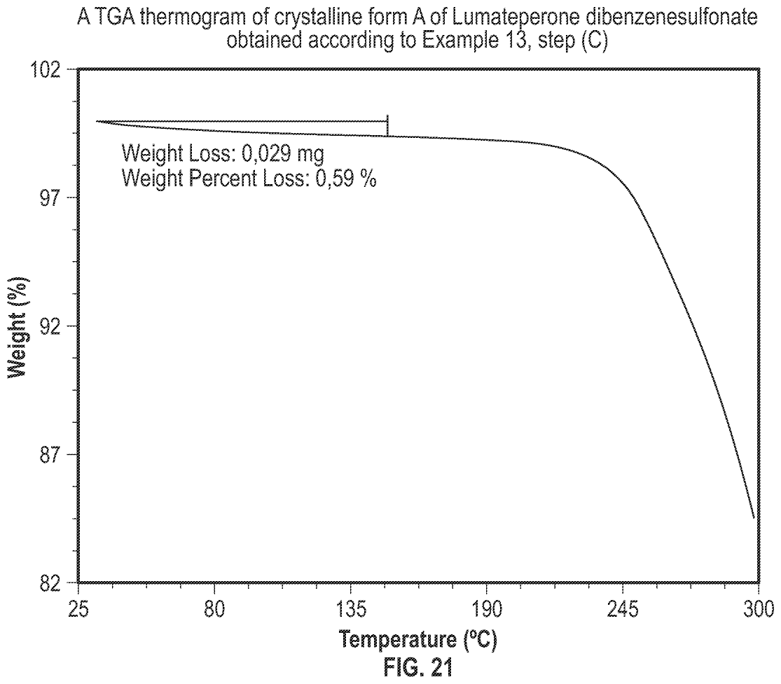

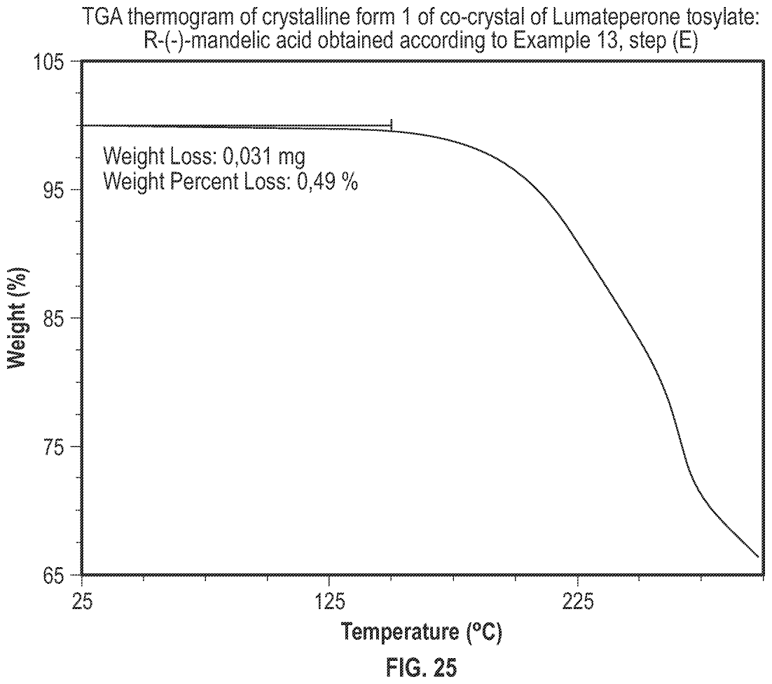

### TGA Method (FIGS. 11, 16, 21 and 25)

TG analysis was performed on instruments Discovery TGA (TA Instruments) with a heating rate of 10° C./min and under nitrogen flow of 50 mL/min. Standard aluminum open pan was used, sample mass was 3-7 mg.

### Solid state NMR (“ssNMR”) Method for Co-Crystal of Lumateperone Dibenzenesulfonate

The spectra were measured at 11.7 T using a Bruker Avance III HD 500 US/WB NMR spectrometer (Karlsruhe, Germany, 2013). The ¹³C CP/MAS NMR spectra employing cross-polarization were acquired using the standard pulse scheme at spinning frequency of 11 kHz. The recycle delay was 8 s and the cross-polarization contact time was 2 ms. The strength of spin-locking fields B1(¹³C) expressed in frequency units ω½π=γB1 was 64 kHz. The ¹³C NMR scale was referenced to α-glycine (176.03 ppm). Frictional heating of the spinning samples was offset by active cooling, and the temperature calibration was performed with Pb(NO₃)₂.

The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν½ was less than 3.5 Hz at 250 ms of acquisition time).

### Solid State NMR (“ssNMR”) Method for Co-Crystal of Lumateperone Tosylate:R-(-)-Mandelic Acid

The spectra were acquired on Agilent Technologies NMR System 600 MHZ NMR spectrometer equipped with 3.2 mm NB dual resonance HX MAS probe. Larmor frequency of carbon nuclei was 150.75 MHz. ¹³C NMR chemical shifts are reported relative to TMS (δ0.0 ppm).

### Fourier-Transform Infrared Spectroscopy(“FTIR”) Method

FTIR spectrum was recorded on a Nicolet 6700 interferometer between 4000 cm⁻¹ and 400 cm⁻¹ with resolution of 4 cm⁻¹, in KBr technique. All the spectra were measured in 16 scans.

### Single Crystal X-Ray Diffraction (“SCXRD”) Method for Co-Crystal of Lumateperone Tosylate:R-(-)-Mandelic Acid

Diffraction measurements were performed on an Oxford Diffraction Xcalibur Kappa CCD X-ray diffractometer with graphite-monochromated MoKα (λ=0.71073 Å) radiation. The data sets were collected using the ω scan mode over the 2θ range up to 54°. Programs CrysAlis CCD and CrysAlis RED were employed for data collection, cell refinement, and data reduction. The structure was solved by direct methods and refined using the SHELXS and SHELXL programs, respectively. The structural refinement was performed on |F|² using all data. All calculations were performed using the WinGX crystallographic suite of programs.

### UPLC Methods

### Method Parameters for Compounds 2a, 3a, 4a, 5a, 6a, Iva and 1

| Instrument | Agilent 1290 or equivalent | | |
|---|---|---|---|
| Column & | Waters Acquity BEH C18, 1.7 μm, 100 × 2.1 mm, | | |
| Packing | Part. No. 186002352 | | |
| Mobile Phase A | 0.1% H₃PO₄ in water (v/v) | | |
| Mobile Phase B | Acetonitrile | | |
| | Time | % Eluent | % Eluent |
| Gradient | (min) | A | B |
| | 0.00 | 95 | 5 |
| | 10.00 | 5 | 95 |
| | 12.00 | 5 | 95 |
| | 12.01 | 95 | 5 |
| | 15.00 | 95 | 5 |

| | |
|---|---|
| Injection volume | 1 μl |
| Flow rate | 0.3 ml/min |
| Run time | 15 min |
| Detector | 230 nm for compounds 2a, 3a, 4a, 5a, 6a and 214 nm |
| | for compound IVa and 1 |
| Column | 60° C. |
| temperature | |
| Autosampler | 15° C. |
| temperature | |

### Method Parameters for Compounds 7 and 8

| Instrument | Agilent 1290 or equivalent | | |
|---|---|---|---|
| Column & | Waters Acquity BEH C18, 1.7 μm, 100 × 2.1 mm, | | |
| Packing | Part. No. 186002352 | | |
| Mobile Phase A | 10 mM NaH₂PO₄, pH = 2.0 | | |
| Mobile Phase B | Methanol | | |
| | Time | % Eluent | % Eluent |
| Gradient | (min) | A | B |
| | 0.00 | 95 | 5 |
| | 10.00 | 10 | 90 |
| | 10.01 | 95 | 5 |
| | 13.00 | 95 | 5 |

| | |
|---|---|
| Injection volume | 1 μl |
| Flow rate | 0.4 ml/min |
| Run time | 13 min |
| Detector | 214 nm |
| Column | 30° C. |
| temperature | |
| Autosampler | 15° C. |
| temperature | |

### Method Parameters for Enantiomeric Excess Determination of Compound 7

| | |
|---|---|
| Instrument | Agilent 1290 or equivalent |
| Column & Packing | Daicel Chiralcel OD-H, 5 μm, 250 × 4.6 mm, |
| | Part. No. 14325 |
| Mobile Phase | n-Hexane:2-Propanol:Diethylamine = |
| | 90:10:0.05 |
| Injection volume | 10 μl |
| Flow rate | 1 ml/min |
| Run time | 20 min |
| Detector | 220 nm |
| Column temperature | 35° C. |
| Autosampler temperature | 20° C. |

### Method Parameters for Enantiomeric Excess Determination of Compound 1, Lumateperone Dibenzenesulfonate and Lumateperone Tosylate:R-(-)-Mandelic Acid Co-Crystal

| | |
|---|---|
| Instrument | Agilent 1290 or equivalent |
| Column & Packing | Daicel Chiralpak AD-H, 5 μm, 250 × 4.6 mm, |
| | Part. No. 19325 |
| Mobile Phase | n-Hexane:Ethanol:Diethylamine = |
| | 75:25:0.1 |
| Injection volume | 10 μl |
| Flow rate | 1 ml/min |
| Run time | 20 min |
| Detector | 232 nm |
| Column temperature | 35° C. |
| Autosampler temperature | 20° C. |

### Method Parameters for 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one and 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one dibenzenesulfonate

| Instrument | Agilent 1290 or equivalent | | |
|---|---|---|---|
| Column & | Waters Acquity BEH C18, 1.7 μm, 100 × 2.1 mm, | | |
| Packing | Part. No. 186002352 | | |
| Mobile Phase A | 0.1% H₃PO₄ in water (v/v) | | |
| Mobile Phase B | Acetonitrile | | |
| | Time | % Eluent | % Eluent |
| Gradient | (min) | A | B |
| | 0.00 | 95 | 5 |
| | 10.00 | 5 | 95 |
| | 12.00 | 5 | 95 |
| | 12.01 | 95 | 5 |
| | 15.00 | 95 | 5 |

| | |
|---|---|
| Injection volume | 1 μl |
| Flow rate | 0.3 ml/min |
| Run time | 15 min |
| Detector | 214 nm |
| Column | 60° C. |
| temperature | |
| Autosampler | 15° C. |
| temperature | |
| Diluent | Acetonitrile:H₂O = 50:50 |
| Needle Wash | Acetonitrile |
| Solvent | |

### Method Parameters for Co-Crystal of Lumateperone Tosylate:R-(-)-Mandelic Acid

| Instrument | Agilent 1290 or equivalent | | |
|---|---|---|---|
| Column & | Waters Acquity BEH C18, 1.7 μm, 100 × 2.1 mm, | | |
| Packing | Part. No. 186002352 | | |
| Mobile Phase A | 0.1% H₃PO₄ in water (v/v) | | |
| Mobile Phase B | Acetonitrile | | |
| | Time | % Eluent | % Eluent |
| Gradient | (min) | A | B |
| | 0.00 | 95 | 5 |
| | 6.00 | 68 | 32 |
| | 13.00 | 5 | 95 |
| | 15.00 | 5 | 95 |
| | 15.01 | 95 | 5 |
| | 18.00 | 95 | 5 |

| | |
|---|---|
| Injection volume | 1 μl |
| Flow rate | 0.450 ml/min |
| Run time | 18 min |
| Detector | 214 nm |
| Column temperature | 30° C. |
| Autosampler | 15° C. |
| temperature | |
| Diluent | Acetonitrile:H₂O = 50:50 |
| Needle Wash | Acetonitrile |
| Solvent | |

### EXAMPLES

Lumateperone can be prepared by any known method, for example as disclosed in Example 9 of U.S. Pat. No. 8,309,722.

### Example 1: Preparation of Form A of Lumateperone Besylate

### Procedure A

Lumateperone base (0.50 g; 1.27 mmol) was dissolved in acetone (0.8 mL) at about 50° C. Benzenesulfonic acid (0.25 g; 1.60 mmol; 1.3 eq) was dissolved in acetone/IPA 4:1 (2 mL) at room temperature and added drop wise to the base solution at about 50° C. The reaction mixture was stirred at about 50° C. for 30 minutes, cooled, under stirring, over 40 minutes to room temperature and stirred for 1 hour, then further cooled, under stirring, over 120 minutes to about 5° C. and stirred for 30 minutes. The precipitate was filtered and washed with acetone (0.5 mL). The obtained solid was analysed by XRPD, DSC and TGA. The XRPD pattern is presented in FIG. 1 and the DSC and TGA thermograms are presented in FIG. 2.

### Example 2: Preparation of Crystalline Form 1 of Lumateperone Tosylate:R-(-)-Mandelic Acid

### Procedure A

Lumateperone tosylate (1.0 gram) was dissolved in acetone (50 mL) and evaporated by rotary evaporator at 20 mbar and 50° C. Obtained amorphous form of Lumateperone tosylate (0.025 grams; 0.044 mmol) and R-(-)-Mandelic acid (0.007 grams; 0.046 mmol) were suspended in toluene (0.2 mL) at RT (25° C.). Suspension was stirred during 2 days at RT (25° C.). Suspension was filtered and analysed by XRPD and the XRPD pattern is presented in FIG. 3.

### Procedure B

Lumateperone tosylate Form A (0.05 g; 0.088 mmol) and R-(-)-Mandelic acid (0.013 g; 0.085 mmol) were subjected to ball mill milling by agate ball mill (2 agate balls ϕ=7 mm, frequency: 25 Hz, time: 60 min) with the addition of toluene (0.1 mL). Obtained solid was analysed by XRPD and identified as form 1.

### Example 3: Preparation of 6-bromo-2,3,4,5-tetrahydro-1H-pyrido[4,3-b]indole hydrochloride

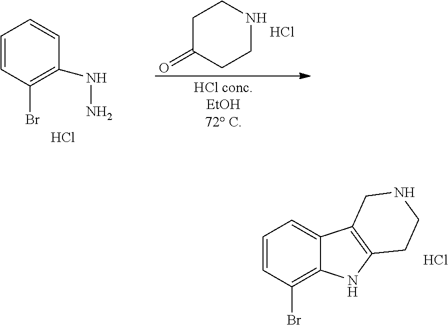 [SMILES: NNc1ccccc1Br ; O=C1CCNCC1 ; Cl ; Cl ; Brc1cccc2c3c([nH]c12)CCNC3 ; Cl]

(2-Bromophenyl)hydrazine hydrochloride (100 grams; 447 mmol; 1 molEq), piperidin-4-one hydrochloride (73 grams; 538.39 mmol; 1.2 molEq) and ethanol (700 mL) were charged in 1 L reactor. Concentrated hydrochloric acid (100 mL; 1184 mmol; 2.65 molEq) was added dropwise. Resulting suspension was heated to reflux overnight. Reaction mixture was cooled down to 0° C. and stirred 2 hours. Product was filtered off, washed with cold ethanol (5×100 ml) and dried in vacuo. Crude 6-bromo-2,3,4,5-tetrahydro-1H-pyrido[4,3-b]indole hydrochloride was isolated as light brown solid in 76% yield (116.37 grams; 83.66% assay; 96.86 Area % UPLC purity). m/z=251 (positive).

### Example 4: Preparation of benzyl 6-bromo-3,4-dihydro-1H-pyrido[4,3-b]indole-2(5H)-carboxylate (2a)

### Procedure A

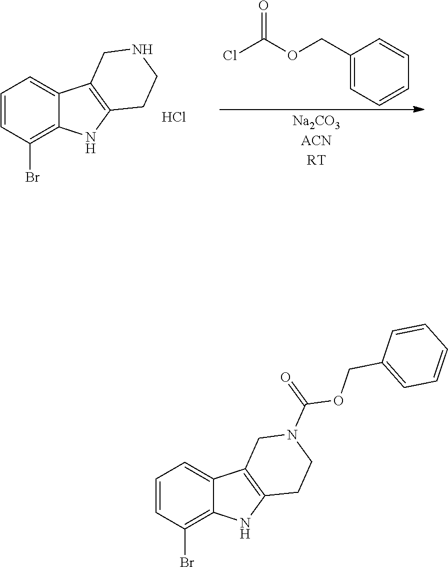 [SMILES: Brc1cccc2c3c([nH]c12)CCNC3 ; Cl ; O=C(Cl)OCc1ccccc1 ; O=C(OCc1ccccc1)N1CCc2[nH]c3c(Br)cccc3c2C1]

6-Bromo-2,3,4,5-tetrahydro-1H-pyrido[4,3-b]indole hydrochloride (234 grams; 82.20% assay; 669 mmol; 1 molEq), sodium carbonate (258.8 grams; 2.442 mol; 3.65 molEq) and acetonitrile (2.34 L) were charged in 6 L reactor. Benzyl chloroformate (233 mL; 1.629 mol; 2.43 molEq) was added dropwise over 1 hour. Reaction mixture was stirred 2 hours at room temperature. Water (2.34 L) was added dropwise over 1 hour and the resulting mixture was stirred 2 hours at RT. Obtained solid was filtered off, washed with 2×468 mL ACN/H₂O (1/1) and with water (2×468 mL) and slurried in water (2.69 L) for 2 h at RT. Product was filtered off, washed with water (2×468 mL) and dried in vacuo. Crude benzyl 6-bromo-3,4-dihydro-1H-pyrido[4,3-b]indole-2(5H)-carboxylate was isolated as white solid in 90% yield (234.37 grams; 98.84% assay; 99.27 Area % UPLC purity). ¹H NMR (500 MHZ, DMSO-d₆) δ11.15 (s, 1H), 7.43-7-30 (m, 6H), 7.25 (d, J=7.7 Hz, 1H), 6.92-6.89 (m, 1H), 5.15 (s, 2H), 4.63-4.60 (m, 2H), 3.81-3.78 (m, 2H), 2.85-2.83 (m, 2H). m/z=385 (positive). Obtained solid was analyzed by XRD, a crystalline form was obtained and the XRPD is presented in FIG. 4.

### Procedure B

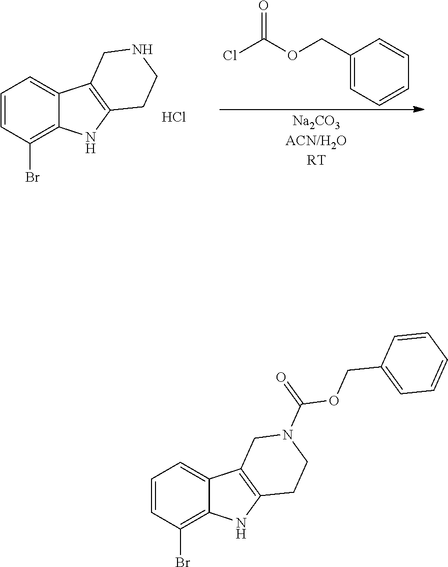 [SMILES: Brc1cccc2c3c([nH]c12)CCNC3 ; Cl ; O=C(Cl)OCc1ccccc1 ; O=C(OCc1ccccc1)N1CCc2[nH]c3c(Br)cccc3c2C1]

6-Bromo-2,3,4,5-tetrahydro-1H-pyrido[4,3-b]indole hydrochloride (5 grams; 79.74% assay; 13.86 mmol; 1 molEq), sodium carbonate (5.53 grams; 52.17 mol; 3.76 molEq) and 75 mL of acetonitrile/water (1/1) were charged in 100 ml flask. Benzyl chloroformate (4970 μL; 34.82 mol; 2.51 molEq) was added dropwise over 2 minutes. Reaction mixture was stirred 2 hours at room temperature. Obtained solid was filtered off, washed with 2×10 mL ACN/H₂O (1/1) and with water (2×10 mL) and dried over 1 h (40° C., 10 mbar). Resulting solid was slurried in water (50 mL) for 2 h at RT. Product was filtered off, washed with water (3×10 mL) and dried in vacuo. Crude benzyl 6-bromo-3,4-dihydro-1H-pyrido[4,3-b]indole-2(5H)-carboxylate was isolated as white solid in 84% yield (4.46 grams; 99.96% assay; 99.36 Area % UPLC purity). ¹H NMR (500 MHZ, DMSO-d₆) δ11.15 (brs, 1H), 7.45-7-30 (m, 6H), 7.25 (d, J=7.7 Hz, 1H), 6.92-6.89 (m, 1H), 5.15 (s, 2H), 4.65-4.60 (m, 2H), 3.81-3.78 (m, 2H), 2.85-2.83 (m, 2H). Obtained solid was analyzed by XRD, a crystalline form was obtained and the XRPD is presented in FIG. 4a.

### Example 5: Preparation of benzyl 6-bromo-5-(2-(methylamino)-2-oxoethyl)-3,4-dihydro-1H-pyrido[4,3-b]indole-2(5H)-carboxylate (3a)

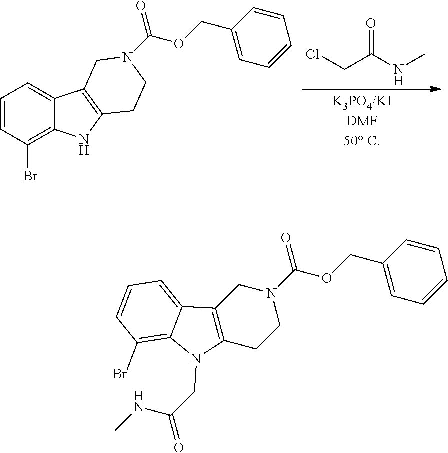

Benzyl 6-bromo-3,4-dihydro-1H-pyrido[4,3-b]indole-2(5H)-carboxylate (228 grams; 98.84% assay; 585 mmol; 1 molEq), potassium iodide (117.95 grams; 710.18 mmol; 1.2 molEq) and 2-chloro-N-methylacetamide (190.93 grams; 1.78 mol, 3 molEq) were suspended in DMF (1.14 L). Potassium phosphate (376.88 grams; 1.78 mol; 3 molEq) was added and the resulting mixture was heated to 50° C. and stirred overnight. Water (1.14 L) was added dropwise at 50° C. over 50 minutes. Resulting suspension was stirred at 50° C. over 30 minutes, cooled to RT over 1.5 hours and stirred at RT 30 minutes. Obtained solid was filtered off, washed twice with 455 mL of DMF/H₂O (1/2) and twice with 455 mL of water and dried in vacuo to obtain 274.98 grams of solid. Crude product (269 grams) was slurried in water (2.69 L) for 2 hours at RT and filtered off. Wet crystals were washed with water (3×400 mL) and dried in vacuo. Benzyl 6-bromo-5-(2-(methylamino)-2-oxoethyl)-3,4-dihydro-1H-pyrido[4,3-b]indole-2(5H)-carboxylate was isolated as off white solid in 86% crude yield (229.07 grams; 97.86 Area % UPLC purity). ¹H NMR (500 MHz, DMSO-d₆) δ8.01-7.98 (m, 1H), 7.47 (brs, 1H), 7.40-7.36 (m, 4H), 7.33-7.30 (m, 1H), 7.26 (d, J=7.7 Hz, 1H), 6.94-6.91 (m, 1H), 5.14 (s, 2H), 5.06 (brs, 2H), 4.54-4.60 (m, 2H), 3.80 (brs, 2H), 2.73-2.70 (m, 2H), 2.62 (d, J=4.4 Hz, 3H). m/z=456 (positive). Obtained solid was analyzed by XRD, a crystalline form was obtained and the XRPD pattern is presented in FIG. 5.

### Example 6. Preparation of benzyl 3-methyl-2-oxo-2,3,9,10-tetrahydro-1H-pyrido[3′,4′:4,5]pyrrolo[1,2,3-de]quinoxaline-8(7H)-carboxylate (4a)

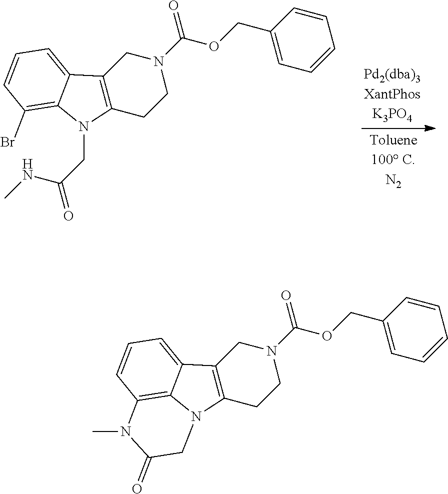 [SMILES: CNC(=O)Cn1c2c(c3cccc(Br)c31)CN(C(=O)OCc1ccccc1)CC2 ; CN1C(=O)Cn2c3c(c4cccc1c42)CN(C(=O)OCc1ccccc1)CC3]

Benzyl 6-bromo-5-(2-(methylamino)-2-oxoethyl)-3,4-dihydro-1H-pyrido[4,3-b]indole-2(5H)-carboxylate (50 grams; 86.27% assay; 94.53 mmol; 1 molEq), potassium phosphate (69.8 grams; 328.7 mmol; 3.5 molEq), XantPhos (951 mg; 1.64 mmol; 0.017 molEq) and palladium tris dibenzylideneacetone (502 mg; 0.55 mmol; 0.0058 molEq) were charged in 1 L reactor under nitrogen atmosphere. Toluene (600 mL) was added, the resulting mixture was heated to 100° C. and was stirred at given temperature for 38 hours. Reaction mixture was cooled to 80° C., water (600 mL) was added and it was stirred at 80° C. for 30 minutes. Resulting two layer solution was filtered over diatomaceous earth (5.0 grams). Diatomaceous earth was washed with toluene (2×25 mL). Layers were separated and organic layer was concentrated in vacuo 60° C. to volume of 250 mL. Product crystallized and the resulting suspension was cooled down to 22° C. over 1 hour and was stirred 1 hour at 22° C. Crystals were filtered off, washed with toluene (2×50 mL) and were dried in vacuo. Crude benzyl 3-methyl-2-oxo-2,3,9,10-tetrahydro-1H-pyrido[3′,4′:4,5]pyrrolo[1,2,3-de]quinoxaline-8(7H)-carboxylate was isolated in 86% yield (31.1 grams; 97.82% assay; 99.21 Area % UPLC purity). ¹H NMR (500 MHZ, DMSO-d₆) δ7.38 (brs, 4H), 7.33-7.31 (m, 1H), 7.15 (d, J=7.5 Hz, 1H), 6.96-6.93 (m, 1H), 6.73 (d, J=7.5 Hz, 1H), 5.14 (s, 2H), 4.98 (brs, 2H), 4.66-4.61 (m, 2H), 3.79 (brs, 2H), 3.35 (s, 3H), 2.81-2.78 (m, 2H). m/z=376 (positive). Obtained solid was analyzed by XRD, a crystalline form was obtained and the XRPD pattern is presented in FIG. 6.

### Example 7. Preparation of benzyl 3-methyl-2,3,9,10-tetrahydro-1H-pyrido[3′,4′:4,5]pyrrolo[1,2,3-de]quinoxaline-8(7H)-carboxylate (5a)

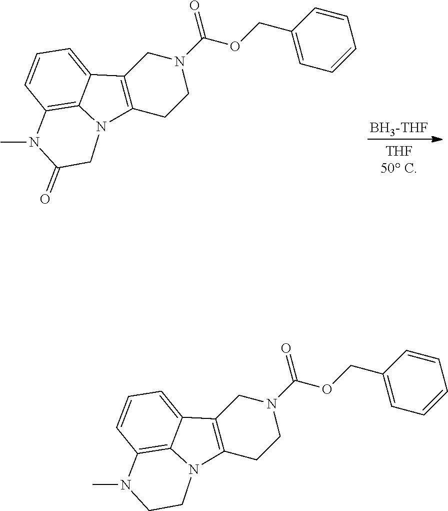 [SMILES: CN1C(=O)Cn2c3c(c4cccc1c42)CN(C(=O)OCc1ccccc1)CC3 ; CN1CCn2c3c(c4cccc1c42)CN(C(=O)OCc1ccccc1)CC3 ; "BH3-THF"]

Benzyl 3-methyl-2-oxo-2,3,9, 10-tetrahydro-1H-pyrido[3′,4′:4,5]pyrrolo[1,2,3-de]quinoxaline-8(7H)-carboxylate (40.0 grams, 106.55 mmol; 1 molEq) was charged into autoclave. Anhydrous THF (134 mL) was added. The resulting suspension was cooled to 0-5° C. and BH₃×THF (266 mL; 1M; 266 mmol; 2.5 molEq) was added in a dropwise manner, over period of 30 minutes. Resulting suspension was heated to 50° C. and was stirred at given temperature for 3 hours. Reaction mixture was cooled down to 0-5° C. Methanol (45 mL) was added in a dropwise manner over 15 minutes. Water (500 mL) was added in a dropwise manner over 1 hour. Suspension was stirred at 0-5° C. for 30 minutes. Crystals were filtered off, washed with 2×50 mL THF/water (1/2) and dried in vacuo. Crude benzyl 3-methyl-2,3,9,10-tetrahydro-1H-pyrido[3′,4′:4,5]pyrrolo[1,2,3-de]quinoxaline-8(7H)-carboxylate was isolated in 92% yield (35.6 grams; 99.87 Area % UPLC purity): ¹H NMR (500 MHz, DMSO-d₆) δ7.38 (brs, 4H), 7.34-7.31 (m, 1H), 6.80 (brs, 2H), 6.28-6.27 (m, 1H), 5.14 (s, 2H), 4.61-4.58 (m, 2H), 4.12-4.11 (m, 2H), 3.79 (brs, 2H), 3.33-3.32 (m, 2H), 2.88 (s, 3H), 2.78-2.76 (m, 2H). m/z=362 (positive). Obtained solid was analyzed by XRD, a crystalline form was obtained and the XRPD figure is presented in FIG. 7.

### Example 8. Preparation of benzyl 3-methyl-2,3,6b,7,10,10a-hexahydro-1H-pyrido[3′,4′:4,5]pyrrolo[1,2,3-de]quinoxaline-8(9H)-carboxylate (6a)

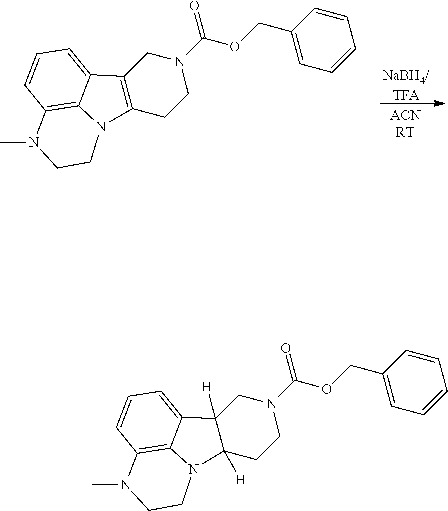 [SMILES: CN1CCn2c3c(c4cccc1c42)CN(C(=O)OCc1ccccc1)CC3 ; "NaBH4/TFA" ; CN1CCN2c3c(cccc31)C1CN(C(=O)OCc3ccccc3)CCC12]

Benzyl 3-methyl-2,3,9,10-tetrahydro-1H-pyrido[3′,4′:4,5]pyrrolo[1,2,3-de]quinoxaline-8(7H)-carboxylate (30.0 g, 83 mmol; 1 molEq) was charged into a 1 L reactor. Acetonitrile (150 mL) was added. The resulting suspension was cooled to −5° C. and TFA (75 mL, 979 mmol; 12 molEq) was added under nitrogen atmosphere. Sodium borohydride (7.2 grams, 190.90 mmol; 2.3 molEq) was added at −5° C. in 6 portions, over 1 hour. Resulting mixture was stirred 3 hours at 25° C. After 3 hours reaction mixture was cooled down to −5° C. Methanol (50 mL) was added in a dropwise manner over 15 minutes. Aqueous solution of NaOH, (2 M; 420 mL) was added in a dropwise manner, over 45 minutes, and pH was set to 9.3. Resulting mixture (lightly yellow) was stirred at 25° C. overnight and pH was corrected to 9.3 with 30 mL 2M NaOH (aq). Reaction mixture was concentrated in vacuo to the volume of 400 mL. Dichloromethane (300 mL) was added and layers were separated. Aqueous layer was additionally extracted with DCM (300 mL). Combined organic layer was evaporated to dryness. Crude benzyl 3-methyl-2,3,6b,7,10,10a-hexahydro-1H-pyrido[3′,4′:4,5]pyrrolo[1,2,3-de]quinoxaline-8(9H)-carboxylate was isolated as brown oil in 103% crude yield (30.9 grams; 98.63 Area % UPLC purity). ¹H NMR (500 MHZ, DMSO-d₆) δ7.39-7.30 (m, 4H), 6.54-6.50 (t, J=7.7 Hz, 1H), 6.44 (brs, 1H), 6.35 (d, J=7.7 Hz, 1H), 5.12-5.08 (m, 2H), 3.91 (brs, 1H), 3.71-3.67 (m, 1H), 3.46-3.41 (m, 1H), 3.35-3.25 (m, 3H), 3.13 (brs, 1H), 3.09-3.04 (m, 1H), 2,78 (m, 4H), 2.75-2.71 (m, 1H), 1.91-1.86 (m, 1H), 1.79-1.72 (m, 1H). m/z=364 (positive).

### Example 9. Preparation of 3-methyl-2,3,6b,7,8,9,10,10a-octahydro-1H-pyrido[3′,4′:4,5]pyrrolo[1,2,3-de]quinoxaline (rac-7)

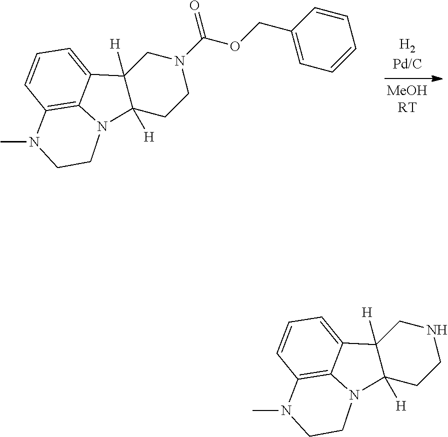 [SMILES: CN1CCN2c3c(cccc31)C1CN(C(=O)OCc3ccccc3)CCC12 ; CN1CCN2c3c(cccc31)C1CNCCC12]

Benzyl 3-methyl-2,3,6b,7, 10,10a-hexahydro-1H-pyrido[3′,4′:4,5]pyrrolo[1,2,3-de]quinoxaline-8(9H)-carboxylate (40.04 grams; 110.17 mmol; 1 molEq) was dissolved in MeOH (400 mL). 4.04 g of Palladium on charcoal (5%, wet; 55.4 wt% H₂O) was added and the resulting mixture was hydrogenated at 5 bar of H₂ at room temperature over 4 hours. Reaction mixture was filtered off over diatomaceous earth (10 grams) and evaporated to dryness. Crude 3-methyl-2,3,6b,7,8,9,10,10a-octahydro-1H-pyrido[3′,4′:4,5]pyrrolo[1,2,3-de]quinoxaline was isolated in 95.4% crude yield (24.08 grams; 97.37 Area % UPLC purity). ¹H NMR (600 MHZ, CDCl₃) δ6.68 (t, J=7.6 Hz, 1H), 6.54 (d, J=7.6 Hz, 1H), 6.44 (d, J=7.6 Hz, 1H), 3.63-3.59 (m, 1H), 3.37-3.30 (m, 3H), 3.12-3.04 (m, 2H), 2.89-2.85 (m, 6H), 2.68-2.65 (m, 1H), 2.16 (brs, 1H), 1.93-1.90 (m, 1H), 1.66-1.83 (m, 1H). m/z=230 (positive).

### Example 10. Preparation of (6bR,10aS)-3-methyl-2,3,6b,7,8,9,10,10a-octahydro-1H-pyrido[3′,4′:4,5]pyrrolo[1,2,3-de]quinoxaline (7) and conversion to Lumateperone

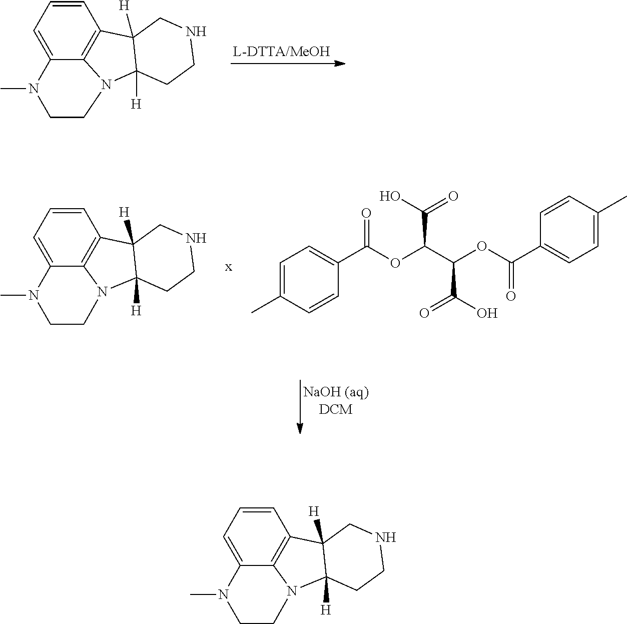

### Procedure A

Racemic 3-methyl-2,3,6b,7,8,9,10,10a-octahydro-1H-pyrido[3′,4′:4,5]pyrrolo[1,2,3-de]quinoxaline (14.2 grams; 61.92 mmol; 1 molEq) was dissolved in methanol (177 ml) and added dropwise to the solution of (−)—O,O′-Di-p-toluoyl-L-tartaric acid (35.5 grams; 91.88 mmol; 1.5 molEq) in methanol (177 ml), under nitrogen atmosphere. Resulting mixture was stirred at room temperature over 1 hour. Obtained solid was filtered off and washed twice with methanol (70 ml) and dried in vacuo to obtain 14.38 g of dry salt (8). ¹H NMR (500 MHZ, DMSO-d₆) δ9.31 (brs, 1H), 7.84 (d, J=7.9 Hz, 4H), 7.31 (d, J=7.9 Hz, 4H), 6.56 (t, J=7.7 Hz, 1H), 6.40-6.38 (m, 2H), 5.63 (s, 2H), 3.46-3.42 (m, 1H), 3.30-3.27 (m, 2H), 3.24-3.21 (m, 1H), 3.17-3.12 (m, 1H), 3.08-3.03 (m, 2H), 2.86-2.80 (m, 1H), 2.79 (s, 3H), 2.64-2.60 (m, 1h), 2.43-2.39 (m, 1H), 2.36 (s, 6H), 1.98-1.91 (m, 2H). Obtained solid was analyzed by XRD, a crystalline form was obtained and the XRPD pattern is presented in FIG. 5. Portion of obtained solid (13.3 grams) was suspended in dichloromethane (133 ml) and water (133 ml) and pH was set to 12.5 using 2M aqueous NaOH solution. Layers were separated and organic layer was washed with water (133 ml) and evaporated to dryness. Crude (6bR, 10aS)-3-methyl-2,3,6b,7,8,9,10,10a-octahydro-1H-pyrido[3′,4′:4,5]pyrrolo[1,2,3-de]quinoxaline was isolated as tanned oil in 37% yield (4.95 grams; 99.81 Area % UPLC purity; 98% ee). ¹H NMR (600 MHZ, CDCl₃) δ6.68 (t, J=7.6 Hz, 1H), 6.54 (d, J=7.6 Hz, 1H), 6.43 (d, J=7.6 Hz, 1H), 3.63-3.59 (m, 1H), 3.37-3.30 (m, 3H), 3.14-3.05 (m, 2H), 2.91-2.85 (m, 6H), 2.68-2.65 (m, 1H), 2.42 (brs, 1H), 1.94-1.90 (m, 1H), 1.66-1.83 (m, 1H).

### Procedure B

(−)—O,O′-Di-p-toluoyl-L-tartaric acid (2.53 grams; 6.54 mmol; 1.5 molEq) was dissolved in 25 mL methanol/water (3/1) at RT under nitrogen atmosphere. Resulting solution was warmed up to 59° C. Solution of racemic 3-methyl-2,3,6b,7,8,9, 10,10a-octahydro-1H-pyrido[3′,4′:4,5]pyrrolo[1,2,3-de]quinoxaline (1 gram; 3.53 mmol; 80.9% assay; 1 molEq) in 25 ml of methanol/water (3/1) was added dropwise to the solution of (−)—O,O′-Di-p-toluoyl-L-tartaric acid at 58° C. Resulting mixture was cooled down to RT over 1 hour and stirred 1 hour at RT. Obtained solid was filtered off and washed twice with methanol (8 ml) and dried in vacuo to obtain of dry salt in 44% yield (980 mg; assay 97.62%, 100 Area % UPLC purity; 100% ee). A portion of (6bR, 10aS)-3-methyl-2,3,6b,7,8,9,10,10a-octahydro- 1 H-pyrido[3′,4′:4,5]pyrrolo[1,2,3-de]quinoxaline salt with (−)—O,O′-Di-p-toluoyl-L-tartaric acid (8) (800 mg; 1.3 mmol; 97.62% assay; 1 molEq) was dissolved in methyl ethyl ketone (8 mL). Water (8 mL) was added and pH was set to 12.4 using 2M NaOH (aq). Layers were separated and water layer was extracted with another portion of methyl ethyl ketone (10 mL). Combined organic layers were dried over anhydrous sodium sulphate and volume was reduced (in vacuo) to 5 mL. Remaining solution was charged to a sealed tube. Potassium iodide (430 mg; 2.6 mmol; 2 molEq), potassium acetate (255 mg; 2.6 mmol; 2 molEq) and 4-chloro-4-fluorobutyrophenone (520 mg; 2.6 mmol; 2 molEq) were added. Resulting mixture was heated at 110° C. overnight. Water (10 mL) and methyl ethyl ketone (5 mL) was added to the cooled reaction mixture. Layers were separated and combined organic layers were evaporated to dryness yielding Lumateperone as tanned oil (785 mg; 43 Area % UPLC purity).

### Example 11. Preparation of (6bR,10aS)-3-methyl-2,3,6b,7,8,9,10,10a-octahydro-1H-pyrido[3′,4′:4,5]pyrrolo[1,2,3-de]quinoxaline (2R,3R)-2,3-bis((4-methylbenzoyl)oxy)succinate (8)

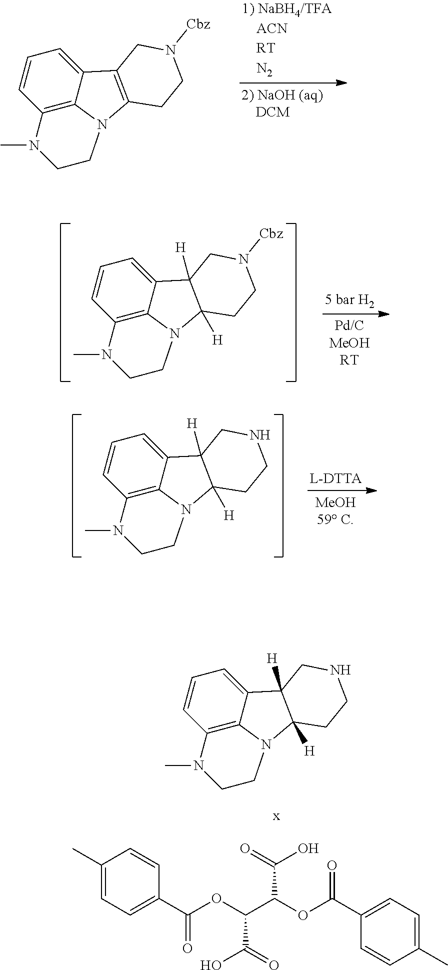

Benzyl 3-methyl-2,3,9,10-tetrahydro-1H-pyrido[3′,4′:4,5]pyrrolo[1,2,3-de]quinoxaline-8(7H)-carboxylate (30.0 grams, 83 mmol; 1 molEq) was charged into 1 L reactor and acetonitrile (150 mL) was added under nitrogen atmosphere. Resulting suspension was cooled down to −5° C. and trifluoroacetic acid (75 mL; 2.5V) was added (exothermic reaction). Sodium borohydride (7.23 grams, 190.90 mmol; 2.3 molEq) was added in 4 portions while maintaining temperature of the reaction mixture at −5° C. Resulting mixture was gradually heated to room temperature over 1 hour and stirred 1 h at 25° C. After reaction completion, mixture was cooled down to −5° C.and MeOH (50 mL) was added in a dropwise manner over 15 minutes (exothermic reaction). Aqueous solution of 2M NaOH (420 mL) was added dropwise over 45 minutes and pH was set to 11.3. The solution was concentrated to ⅔ of the starting volume in vacuo. Dichloromethane (400 mL) was added and layers were separated. Aqueous layer was extracted with dichloromethane (200 mL). Combined organic layers were washed with water (400 mL) and concentrated in vacuo to ½ of the starting volume, methanol was then added (300 mL), and the solution was concentrated in vacuo to 200 mL. Additional 188 mL MeOH was added and the solution was charged in 600 mL autoclave. Catalyst (2.9 grams, 5% Pd/C; 50 wt % H2O) was added and the resulting mixture was hydrogenated at 5 bar of H2 pressure at room temperature over 4 hours. Reaction mixture was filtered over Celite (5 grams) and Celite was washed with MeOH (2×10 mL). Filtrate (302 mL) was diluted to 425 mL. (−)—O,O′-Di-p-toluoyl-L-tartaric acid (111 mmol; 1.3 eq, 42.96 grams) was charged into the reactor and 425 mL of methanol was added. The resulting clear solution was heated to 59° C. ⅓ (142 mL) of the solution of 3-methyl-2,3,6b,7,8,9,10,10a-octahydro-1H-pyrido[3′,4′:4,5]pyrrolo[1,2,3-de]quinoxaline was added dropwise during 45 minutes. The solution was seeded with (6bR,10aS)-3-methyl-2,3,6b,7,8,9,10,10a-octahydro-1H-pyrido[3′,4′:4,5]pyrrolo[1,2,3-de]quinoxaline x L-DTTA (68 mg; 0.11 mmol). The solution became opalescent and was left to stir at 59° C. over 30 minutes. The remaining ⅔ of 3-methyl-2,3,6b,7,8,9,10,10a-octahydro-1H-pyrido[3′,4′:4,5]pyrrolo[1,2,3-de]quinoxaline solution was added dropwise over 90 minutes. The suspension was left to stir for 1 hour at 59° C., and then was cooled to room temperature over 1 hour and stirred at room temperature for 30 minutes. The crystals were filtered off and washed twice with methanol (150 mL) and dried in vacuo. (6bR,10aS)-3-methyl-2,3,6b,7,8,9,10,10a-octahydro-1H-pyrido[3′,4′:4,5]pyrrolo[1,2,3-de]quinoxaline (2R,3R)-2,3-bis((4-methylbenzoyl)oxy)succinate was isolated in 40% yield (20.63 g, 99.88%; >99% ee).

### Example 12. Preparation of Co-Crystal of Lumateperone Tosylate:R-(-)-Mandelic Acid

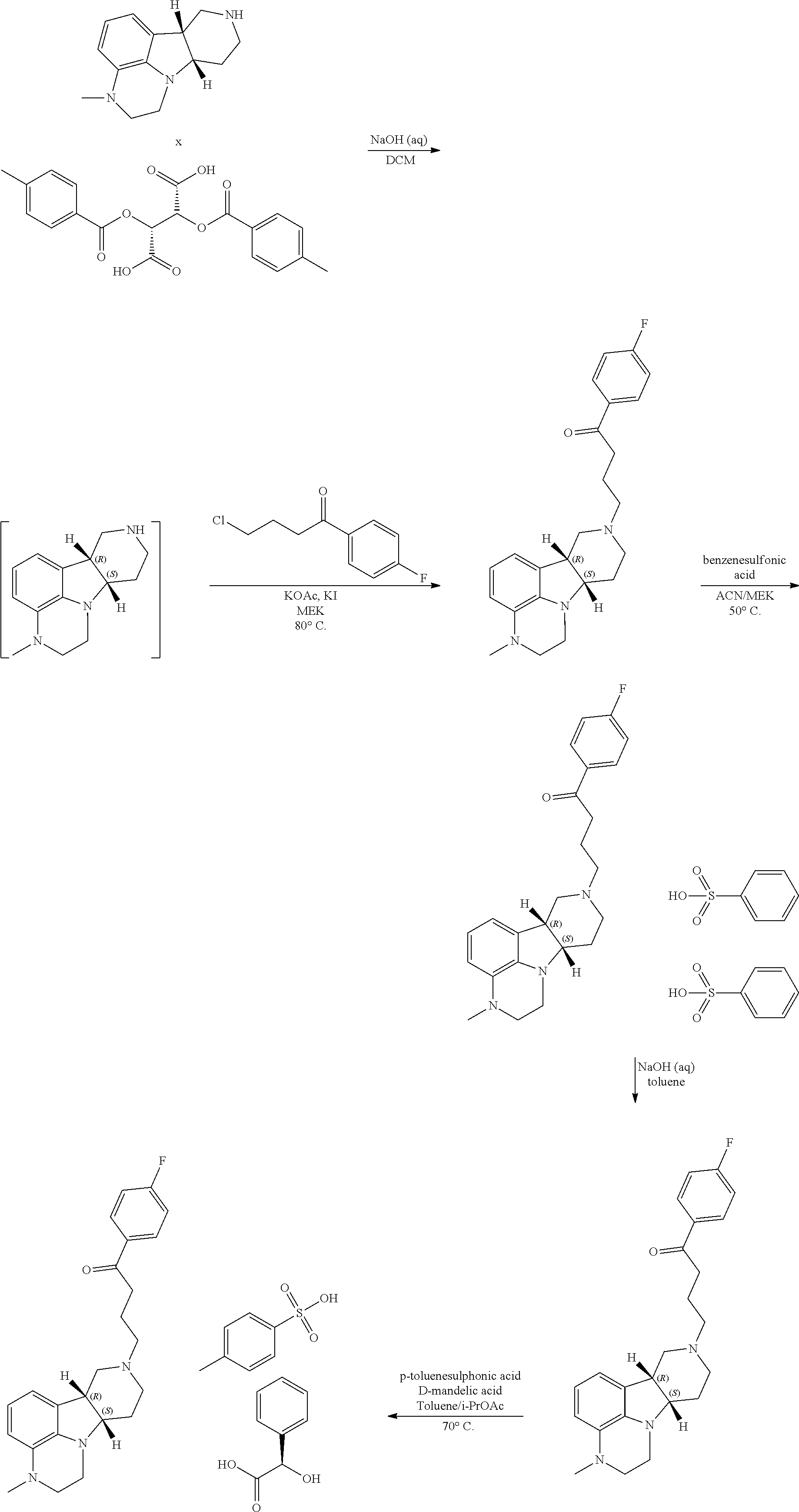

### Step A

(6bR,10aS)-3-methyl-2,3,6b,7,8,9,10, 10a-octahydro-1H-pyrido[3′,4′:4,5]pyrrolo[1,2,3-de]quinoxaline (2R,3R)-2,3-bis((4-methylbenzoyl)oxy)succinate (30.0 g, assay 95%; 46.29 mmol; 1 molEq) was charged into IL reactor and methyl ethyl ketone (300 mL) and water (300 mL) were added, followed by 2.0 M NaOH aq. solution (≈50-55 mL) till pH reached value 12.5. Layers were separated and aqueous layer was extracted with methyl ethyl ketone (2×370 mL). Combined organic layers were washed with saturated aqueous NaCl solution (200 mL) and dried over anhydrous sodium sulphate. Filtered organic layer was concentrated to volume of 140 mL. Potassium iodide (15.4 g; 92.77 mmol; 2 molEq) and KOAc (9.1 g, 92.72 mmol; 2 molEq) were added, followed by 4-chloro-1-(4-fluorophenyl)butan-1-one (18.6 g, 92.70 mmol; 2 molEq) and methyl ethyl ketone (45 mL). Reaction mixture was heated to reflux (80° C.) and was stirred at given temperature for 16 h. Reaction mixture was cooled down to RT. Isopropyl acetate (180 mL) was added, followed by 2.0 M HCl, aqueous solution (300 mL). Layers were separated. To the aqueous layer toluene (600 mL) was added, followed by 2.0 M NaOH aqueous solution (≈400 mL) till pH reached value 13. Resulting mixture was stirred for 30 min at RT. Layers were separated and to the organic layer active charcoal (2.0 g) was added. Mixture was stirred at RT overnight, filtered over pad of celite layer (15.0 g). Celite was washed with toluene (100 mL). Filtrate was evaporated to dryness. Crude 1-(4-fluorophenyl)-4-((6bR, 10aS)-3-methyl-2,3,6b,7,10,10a-hexahydro-1H-pyrido[3′,4′:4,5]pyrrolo[1,2,3-de]quinoxalin-8(9H)-yl)butan-1-one was isolated as brown oil in 81% yield (17.7 g; 83.42% assay; 91.85 Area % UPLC purity; chiral purity: 98.54% ee).

### Step B

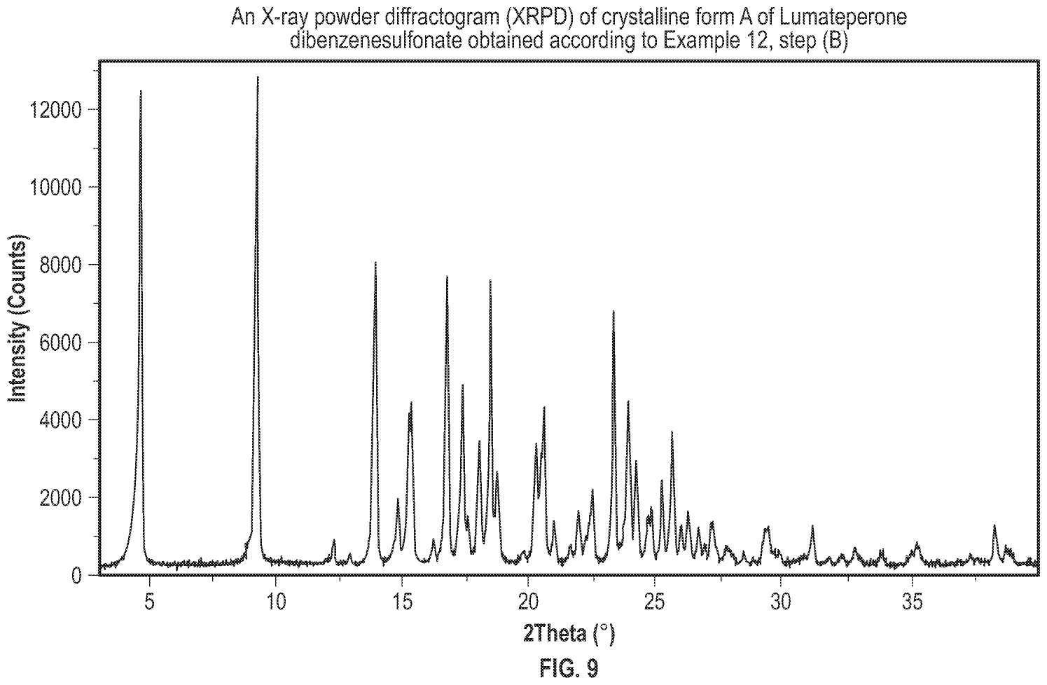

Crude 1-(4-fluorophenyl)-4-((6bR,10aS)-3-methyl-2,3,6b,7,10, 10a-hexahydro-1H-pyrido[3′,4′:4,5]pyrrolo[1,2,3-de]quinoxalin-8(9H)-yl)butan-1-one (1.5 g; 83.42% assay; 3.180 mmol; 1 molEq) was charged into 50 mL three necked flask and acetonitrile (9 mL) was added. Obtained solution was heated to 50° C. and benzenesulfonic acid monohydrate (1.2 g; 6.810 mmol; 2.14 molEq) was added. Methyl ethyl ketone (3 mL) was added and obtained suspension was stirred at 50° C. for 30 min. Suspension was gradually cooled down to RT and was left stirring at RT overnight. Methyl ethyl ketone (9 mL) was added dropwise and stirred at RT for 1 h. Crystals were filtered off, washed with methyl ethyl ketone (2×3 mL) and dried in vacuo until constant mass (40° C., 10 mbar). Crude 1-(4-fluorophenyl)-4-((6bR,10aS)-3-methyl-2,3,6b,7,10,10a-hexahydro-1H-pyrido[3′,4′:4,5]pyrrolo[1,2,3-de ]quinoxalin-8(9H)-yl)butan-1-one dibenzenesulfonate was isolated as off-white solid in 93% yield (2.1 g; 99.38 Area % UPLC purity). m/z=394 (positive) Obtained solid was analyzed by XRD, a crystalline form was obtained and the XRPD is presented in FIG. 9 (chiral purity: 99.90% ee).

### Step C

To 1-(4-fluorophenyl)-4-((6bR,10aS)-3-methyl-2,3,6b,7, 10,10a-hexahydro-1H-pyrido[3′,4′:4,5]pyrrolo[1,2,3-de]quinoxalin-8(9H)-yl)butan-1-one dibenzenesulfonate (1.8 g; 2.536 mmol; 1 molEq) water (30 mL) and toluene (30 mL) were added. Aqueous solution of NaOH (≈6 mL; 2M) was added (pH set to 13.0). Resulting mixture was stirred 30 min at RT. Layers were separated and organic layer was filtered and evaporated to dryness. Crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one was isolated as yellow oil in 97% yield (1.13 g; 90.62% assay 99.42 Area %. UPLC purity; 2.602 mmol, chiral purity: 99.90% ee).

1H NMR (600 MHZ, DMSO-d₆) δ8.05-8.03 (m, 2H), 7.36-7.32 (m, 2H), 6.50 (t, 1H), 6.39 (d, 1H), 6.32 (d, 1H), 3.43-3.40 (m, 1H), 3.30-3.25 (m, 2H), 3.05-3.03 (m, 1H), 2.99 (t, 2H), 2.90-2.86 (m, 1H), 2.78 (s, 3H), 2.73-2.70 (m, 1H), 2.68-2.64 (m, 1H), 2.54-2.50 (m, 1H), 2.32-2.23 (m, 2H), 2.09-2.05 (m, 1H), 1.85-1.73 (m, 4H), 1.67-1.61 (m, 1H); ¹³C NMR (150 MHZ, DMSO-d₆) δ198.94, 165.47, 138.16, 135.28, 134.25, 131.29, 130.13, 120.23, 116.07, 112.74, 109.03, 64.55, 57.63, 56.47, 50.48, 49.18, 44.27, 41.44, 37.57, 36.16, 24.80, 22.16.

### Step D

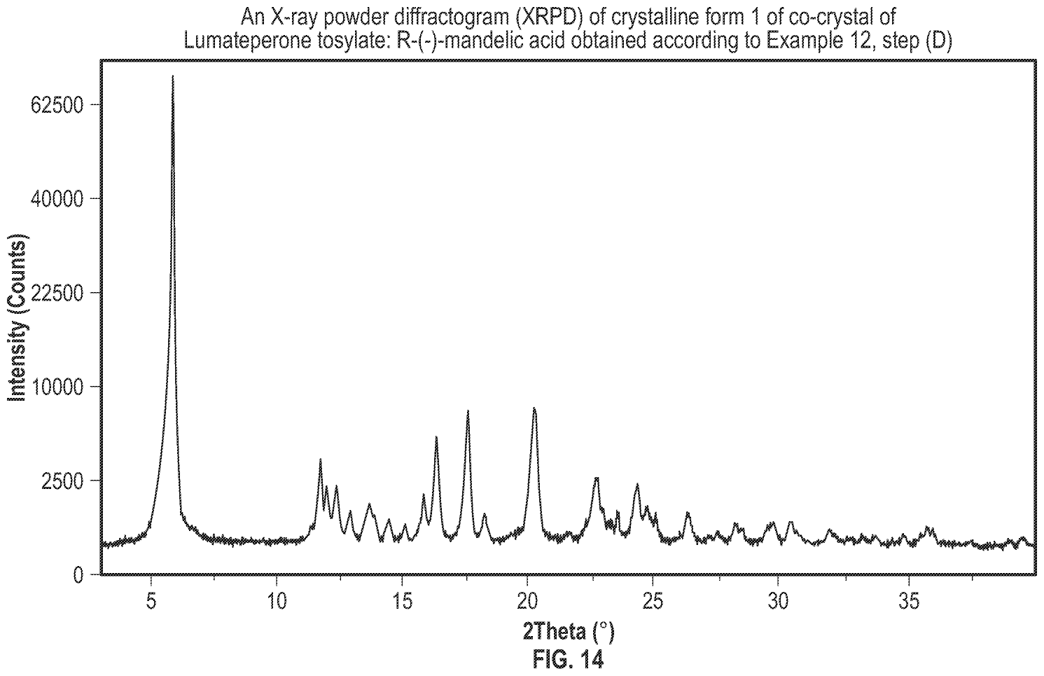

Crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (1.13 g; 90.62% assay; 2.602 mmol; 1 molEq) was charged into a 25 mL, three neck, flask. Toluene (4 mL) and i-propyl acetate (3 mL) were added. To the resulting solution p-toluenesulfonic acid monohydrate (458 mg; 2.408 mmol; 0.93 molEq) and R-(-)mandelic acid (579 mg; 3.804 mmol; 1.5 molEq) were added. Obtained suspension was stirred 30 min at 70° C. under argon atmosphere. Resulting solution was gradually cooled down to 0-5° C. and stirred 1 h. Obtained solid was filtered off, washed with i-propylacetate (2×2mL) and dried in vacuo until constant mass (40° C., 10 mbar). Co-crystal of 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one with p-toluenesulfonic acid and R-(-)mandelic acid was isolated as off white solid in 80% yield (1.5 g; 99.85 Area %). m/z=394 (positive) Obtained solid was analyzed by XRD, a crystalline form was obtained and the XRPD is presented in FIG. 14 (chiral purity: 100% ee).

1H NMR (600 MHZ, DMSO-d₆) δ8.05 (dd, 2H), 7.50 (d, 2H), 7.42 (d, 2H), 7.37-7.33 (m, 4H), 7.29-7.27 (m, 1H), 7.11 (d, 2H), 6.60 (t, 1H), 6.51 (d, 1H), 6.42 (d, 1H), 5.02 (s, 1H), 3.59-3.57 (m, 1H), 3.48-3.30 (m, 5H), 3.21 (brs, 1H), 3.16-3.02 (m, 5H), 2.80 (s, 3H), 2.71 (td, 1H), 2.56 (td, 1H), 2.28 (s, 3H), 2.32-2.27 (m, 1H), 2.13-1.96 (m, 3H); ¹³C NMR (150 MHz, DMSO-d₆) δ197.68, 174.56, 165.61, 145.95, 140.69, 138.26, 137.76, 135.68, 133.58, 131.38, 128.58, 128.10, 127.10, 125.96, 121.07, 116.23, 112.99, 109.83, 72.84, 62.66, 56.02, 53.01, 50.26, 48.23, 44.22, 39.02, 37.48, 35.35, 22.17, 21.24, 18.49.

### Example 13. Preparation of Co-Crystal of Lumateperone Tosylate:R-(-)-Mandelic Acid

### Step A

(6bR,10aS)-3-methyl-2,3,6b,7,8,9,10, 10a-octahydro-1H-pyrido[3′,4′:4,5]pyrrolo[1,2,3-de]quinoxaline (2R,3R)-2,3-bis((4-methylbenzoyl)oxy)succinate (11.0 g, assay 94.53%, 16.98 mmol; 1 molEq) was charged into 500 mL round bottomed flask and dichloromethane (110 mL) and water (110 mL) were added, followed by 2.0 M NaOH aq. solution till pH reached value 12.53. Layers were separated and aqueous layer was additionally extracted with dichloromethane (2×110 mL). Combined organic layers were evaporated to dryness. (6bR,10aS)-3-methyl-2,3,6b,7,8,9,10,10a-octahydro-1H-pyrido[3′,4′:4,5]pyrrolo[1,2,3-de]quinoxaline was isolated as tanned oil in 104% yield (4.70 g; 86.15% assay; 99.23 Area % UPLC purity).

### Step B

To the obtained oil of (6bR,10aS)-3-methyl-2,3,6b,7,8,9,10,10a-octahydro-1H-pyrido[3′,4′:4,5]pyrrolo[1,2,3-de]quinoxaline (4.70 g; 86.15% assay; 17.66 mmol; 1 molEq) potassium iodide (5.93 g, 35.72 mmol; 2 molEq) and KOAc (3.51 g, 35.72 mmol; 2 molEq) were added, followed by 4-chloro-1-(4-fluorophenyl)butan-1-one (7.17 g, 35.73 mmol; 2 molEq) and methyl ethyl ketone (66 mL). Resulting mixture was stirred at 70° C. overnight. After 18 h reaction mixture was cooled down to RT and H₂O (66 mL) was added, followed by 2.0 M NaOH, aq. solution till pH reached 12.6. Layers were separated and organic layer was evaporated to dryness. Crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one was isolated as brown oil in 81% yield (9.96 g; 66.78 Area % UPLC purity; 56.64% assay).

### Step C

To the crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (9.96 g; 56.64% assay; 14.34 mmol; 1 molEq) methyl ethyl ketone (15 mL) and acetonitrile (15 mL) were added. Obtained solution was heated to 50° C. and solution of benzenesulfonic acid monohydrate (5.6 g; 31.78 mmol; 2.22 molEq) in acetonitrile (15 mL) was added dropwise over 30 min. Seeding was introduced and the resulting suspension was stirred at 50° C. over 30 min. It was then gradually cooled down to RT and methyl ethyl ketone was added (45 mL) over 30 min. Suspension was stirred at RT over 1 h. Obtained solid was filtered off, washed with 2×10 mL of acetonitrile/methyl ethyl ketone (1/4) and dried in vacuo until constant mass (40° C., 10 mbar) affording crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one dibenzenesulfonate as off-white solid in 87% yield (8.9 g; 99.61 Area % UPLC purity, chiral purity: 100% ee). m/z=394 (positive) Obtained solid was analyzed by XRD, a crystalline form was obtained and the XRPD is presented in FIG. 18. ¹H NMR (600 MHZ, DMSO-d₆) δ9.13 (brs, 1H), 8.06-8.04 (m, 2H), 7.60-7.59 (m, 4H), 7.40-7.30 (m, 8H), 6.63-6.61 (m, 1H), 6.57-6.56 (m, 1H), 6.51-6.48 (m, 1H), 3.62-3.58 (m, 1H), 3.50-3.32 (m, 5H), 3.28 (brs, 1H), 3.13 (t, 2H), 3.18-3.04 (m, 3H), 2.84 (s, 3H), 2.76-2.72 (m, 1H), 2.60-2.54 (m, 1H), 2.27-2.25 (m, 1H), 2.11-1.98 (m, 3H).

### Step D

To the 1-(4-fluorophenyl)-4-((6bR,10aS)-3-methyl-2,3,6b,7,10, 10a-hexahydro-1H-pyrido[3′,4′:4,5]pyrrolo[1,2,3-de]quinoxalin-8(9H)-yl)butan-1-one dibenzenesulfonate (4 g; 5.635 mmol; 1 molEq) water (50 mL) and toluene (50 mL) were added, followed by 2.0 M NaOH, aq. solution (˜13 mL). The pH was set to ˜13. Layers were separated and organic layer was evaporated to dryness. Crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one was isolated as yellow oil in 102% crude yield (2.34 g; 96.64% assay; 99.47 Area % UPLC purity, (chiral purity: 99.98% ee). m/z=394.2 (positive). ¹H NMR (600 MHZ, CDCl₃) δ8.02-7.99 (m, 2H), 7.14-7.11 (m, 2H), 6.64 (t, 1H), 6.51 (d, 1H), 6.40 (d, 1H), 3.61-3.57 (m, 1H), 3.32-3.25 (m, 2H), 3.20-3.18 (m, 1H), 3.10-3.06 (m, 1H), 2.99-2.96 (m, 2H), 2.86-2.80 (m, 5H), 2.66-2.62 (m, 1H), 2.42-2.34 (m, 2H), 2.26-2.22 (m, 1H), 2.00-1.79 (m, 5H); ¹³C NMR (150 MHZ, CDCl₃) δ198.59, 165.61, 138.00, 134.98, 133.66, 130.70, 130.07, 120.29, 115.58, 112.71, 108.90, 64.56, 57.83, 56.32, 50.66, 48.98, 44.31, 41.72, 37.58, 36.35, 24.98, 21.88.

### Step E

Crude 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (1.17 g; 96.64% assay; 2.873 mmol; 1 molEq) was dissolved in toluene (4.4 mL) and i-PrOAc (3.3 mL). To the obtained solution p-toluenesulfonic acid monohydrate (509 mg; 2.676 mmol; 0.93 molEq) was added, followed by R-(-)mandelic acid (643 mg; 4.226 mmol; 1.47 molEq). Obtained suspension was stirred 30 min at 70° C. under argon atmosphere, and then gradually cooled down to 0-5° C. and stirred at given temperature for 1 h. Precipitate was filtered off, washed with i-PrOAc (2×3 mL) and dried in vacuo until constant mass (10 mbar, 40° C.). Co-crystal of 1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one with p-toluenesulfonic acid and R-(-)mandelic acid was isolated as off white solid in 78% yield (1.6 g; 99.94 Area %). m/z=394 (positive) Obtained solid was analyzed by XRD, a crystalline form was obtained and the XRPD is presented in FIG. 22 (chiral purity: 100% ee).

¹H NMR (600 MHZ, DMSO-d₆) δ8.05 (dd, 2H), 7.50 (d, 2H), 7.42 (d, 2H), 7.38-7.33 (m, 4H), 7.30-7.27 (m, 1H), 7.11 (d, 2H), 6.60 (t, 1H), 6.51 (d, 1H), 6.42 (d, 1H), 5.02 (s, 1H), 3.59-3.56 (m, 1H), 3.48-3.30 (m, 5H), 3.21 (brs, 1H), 3.16-3.02 (m, 5H), 2.80 (s, 3H), 2.71 (td, 1H), 2.56 (td, 1H), 2.28 (s, 3H), 2.32-2.27 (m, 1H), 2.13-1.98 (m, 3H); ¹³C NMR (150 MHZ, DMSO-d₆) δ197.68, 174.56, 165.60, 145.98, 140.69, 138.24, 137.76, 135.68, 133.58, 131.38, 128.59, 128.10, 127.10, 125.96, 121.07, 116.20, 112.99, 109.83, 72.84, 62.66, 56.02, 53.01, 50.26, 48.23, 44.22, 39 .02, 37.48, 35.34, 22.17, 21.24, 18.50.

### Example 14. Preparation of Co-Crystal of Lumateperone Tosylate:R-(-)Mandelic Acid

Form A of Lumateperone tosylate can be prepared according to WO2009/114181. Lumateperone tosylate Form A (0.089 g; 0.16 mmol) and R-(-)mandelic acid (0.023 g, 0.15 mmol) were dissolved in EtOH (3.3 mL) at RT (25° C.). Toluene (6.6 mL) was added drop wise to the solution and it was left to evaporate at RT (25° C.) during 14 days. Obtained crystals were isolated and analyzed by SCXRD.

1. A benzenesulfonate salt of Lumateperone having the formula: 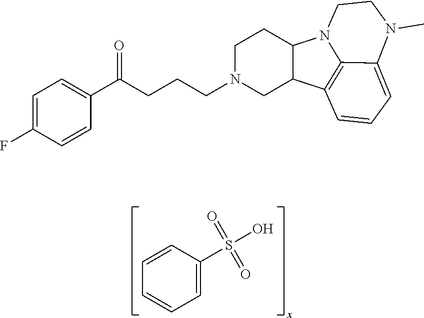
wherein x can be any number between 0.5 and 3.

2. The salt according to claim 1 wherein X is 0.5, 1, 1.5, 2, 2.5 or 3.

3. The salt according to claim 2 wherein X is 1 or 2.

4. The salt according to claim 3, wherein the salt is a dibenzenesulfonate salt of Lumateperone in crystalline form.

5. The crystalline form of dibenzenesulfonate salt of Lumateperone according to claim 4 characterized by data selected from one or more of the following:
(i) an XRPD pattern having peaks at 4.6, 9.2, 13.9, 20.5 and 23.2 degrees 2-theta±0.2 degrees 2-theta;
(ii) an XRPD pattern having peaks at 4.6, 9.2, 13.9, 20.5 and 23.2 degrees 2-theta±0.2 degrees 2-theta and also having one, two, three, four or five additional peaks selected from 15.3, 16.7, 18.0, 22.4 and 25.1 degrees two theta±0.2 degrees two theta;
(iii) an XRPD pattern substantially as depicted in FIG. 1, 9 or 19;
(iv) a solid state ¹³C NMR spectrum substantially as depicted in FIG. 12;
(v) a solid state ¹³C NMR spectrum having peaks at 195.0, 163.5, 119.8 and 57.1 ppm±0.2 ppm;
(vi) a solid state ¹³C NMR spectrum having the following chemical shift absolute differences between said characteristic peaks at 195.0, 163.5, 119.8 and 57.1 ppm±0.2 ppm and a reference peak at 33.5 ppm±0.2 ppm of 161.5, 130.0, 86.3 and 23.6±0.1 ppm;
(vii) FT-IR spectrum substantially as depicted in FIG. 13 or FIG. 22; and
(viii) an FT-IR spectrum having absorptions at 2392, 1638, 1483, 1226, 1122 and 613 cm⁻¹±4 cm⁻¹.

6. The crystalline dibenzenesulfonate salt of Lumateperone according to claim 4 wherein the form is anhydrous.

7. The crystalline dibenzenesulfonate salt of Lumateperone according to claim 4 wherein said crystalline dibenzenesulfonate salt of Lumateperone is isolated.

8. A process for the preparation of crystalline Lumateperone Besylate, comprising crystallisation of Lumateperone Besylate from a solvent comprising one or more polar solvents.

9. The process according to claim 8, wherein the solvent comprises a polar aprotic solvent optionally in combination with a polar protic solvent.

10. The process according to claim 8, wherein the polar aprotic solvent is selected from a nitrile, an ether, a ketone, an ester, or mixtures thereof.

11. The process according to claim 8, wherein the polar protic solvent is an alcohol.

12. The process according to claim 8, wherein a crystalline Form A of Lumateperone Besylate is prepared by crystallisation of Lumateperone Besylate from a solvent mixture comprising acetone and isopropanol.

13. The process according claim 9, wherein the volume ratio of the polar aprotic solvent to polar protic solvent is from about 1:10 to about 10:1.

14. The process according to claim 8, further comprising cooling a solution of Lumateperone besylate in the solvent or solvent mixture.

15. The process according to claim 14, wherein the solution of Lumateperone besylate is cooled to a temperature of about 30° C. to about 70° C.

16. The process according to claim 8 for the preparation of crystalline Lumateperone Besylate, wherein the process comprises:
a) providing Lumateperone in a solution in one or more polar solvents;
b) adding benzenesulfonic acid, optionally in the form of a solution in one or more polar solvents;
c) optionally stirring;
d) optionally cooling; and
e) optionally isolating crystalline dibenzenesulfonate salt of Lumateperone.

17. The process according to claim 16, wherein the solvent comprises at least one polar solvent.

18. The process according to claim 17, wherein the polar solvent is selected from a nitrile, an ether, a ketone, and an ester, or a mixture thereof.

19. The process according to claim 18, wherein the polar solvent includes nitrile and ketone, at a ratio of about 6:1 to about 1:4.

20. The process according to claim 16, comprising cooling a solution of Lumateperone besylate in the solvent or solvent mixture at a temperature of about 40° C. to about 70° C.

21. The process according to claim 16, wherein the crystallization is carried out in the presence of seed crystals of Lumateperone dibesylate.

22. The process according to claim 16 for the preparation of crystalline besylate, wherein the process comprises:
f) providing Lumateperone in acetonitrile at a temperature of about 40° C. to about 60° C.;
g) adding benzenesulfonic acid;
h) adding methyl ethyl ketone to obtain a suspension;
i) optionally stirring the suspension at a temperature of about 40° C. to about 60° C.;
j) optionally cooling to room temperature and stirring; and
k) optionally isolating crystalline dibenzenesulfonate salt of Lumateperone.

23. The process according to claim 16 for preparation of crystalline besylate, wherein the process comprises:
f) providing Lumateperone in a mixture of acetonitrile and ethyl methyl ketone at a temperature of about 40° C. to about 60° C.;
g) adding benzenesulfonic acid, in the form of a solution in acetonitrile;
h) optionally seeding with Lumateperone dibenzenesulfonate seeds;
i) optionally stirring at a temperature of about 40° C. to about 60° C.;
j) optionally cooling to room temperature and stirring;
k) adding methyl ethyl ketone and stirring; and
l) optionally isolating crystalline dibenzenesulfonate salt of Lumateperone.

24. A process for purifying Lumateperone, comprising preparing Lumateperone dibenzenesulfonate according to the process of claim 8 and converting the Lumateperone dibenzenesulfonate to form purified Lumateperone.

25. The process according to claim 24, further comprising converting the purified Lumateperone to a Lumateperone salt.
